SEQUENCE LISTING (77 sequences)

SEQ ID NO: 1 - protein, 735 aa, adeno-associated virus 2 [sequence omitted]

SEQ ID NO: 2 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQTNSGTLQQSRLLFSQAGPTNMSLQAKNWLPGPCYRQQRLSKQANDNNNSNFPWTAATKYHLNGRDSLVNPGPAMASHKDDEEKFFPMHGTLIFGKQGTNANDADLENVMITDEEEIRTTNPVATEQYGTVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 3 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGEPPAAPTSLGSTTMASGGGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTAATKYHLNGRDSLVNPGPAMASHKDDEEKFFPMHGTLIFGKEGTNANNAELENVMITDEEEIRTTNPVATEQYGYVSNNLQNSNTAATTETVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 5 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGTAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 6 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPAKKRLNFGQTGDSDSVPDPQPLGEPPAAPTSLGSTTMASGGGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSASGTVQQSRLLFSQAGPTSMSLQAKNWLPGPCYRQQRLSKQANENNNSNFPWTAATKYHLNGRDSLVNPGPPMASHKDDEEKFFPMHGTLIFGKEGTNANNAELENVMITDEEEIRTTNPVATEQYGNVSNNLQNSNTAPSTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 7 - protein, 735 aa, Adeno-associated virus
MAADGYLPDWLEDTLSEGIRQWWKLKPGPPPPKPAERHKDNSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPAKKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 8 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLNSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGEPPAAPTSLGSTTMASGGGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQNSSGTVQQSRLLFSQAGPTSMSLQAKNWLPGPCYRQQRLSKQASDNNNSNFPWTAATKYHLNGRDSLVNPGPAMASHKDDEEKFFPMHGTLIFGKEGTTANNADLEHVMITDEEEIRTTNPVATEQYGNVSNNLQNSNTGPTTENVNNQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 9 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGEPPAAPTSVGSTTMASGGGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQNSSGTVQQSRLLFSQAGPTSMSLQAKNWLPGPCYRQQRLSKQASDNNNSNFPWTAATKYHLNGRDSLVNPGPAMASHKDDEEKFFPMHGTLIFGKEGTTANNADLEHVMITDEEEIRTTNPVATEQYGNVSNNLQNSNTGPTTENVNNQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 10 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKKDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 11 - protein, 735 aa, Artificial Sequence; identical to SEQ ID NO: 6
  CMRI_10 capsid polypeptide (VP1)

SEQ ID NO: 12 - protein, 737 aa, Artificial Sequence
  CMRI_11 capsid polypeptide (VP1)
MAADGYLPDWLEDTLSEGIRQWWKLKPGPPPPKPAERHKDDSRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPSTTFSPAKFASFITQYSTGQVSVEIEWELQKENSKRWNPEIQYTSNYNKSVNVDFTVDTNGVYSEPRPIGTRYLTRNL

SEQ ID NO: 13 - protein, 737 aa, Artificial Sequence
  CMRI_12 capsid polypeptide (VP1)
MAADGYLPDWLEDNLSEGIRQWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPSTTFSPAKFASFITQYSTGQVSVEIEWELQKENSKRWNPEIQYTSNYNKSVNVDFTVDTNGVYSEPRPIGTRYLTRNL

SEQ ID NO: 14 - protein, 737 aa, Artificial Sequence
  CMRI_13 capsid polypeptide (VP1)
MAADGYLPDWLEDTLSEGIRQWWKLKPGPPPPKPAERHKDDSRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPSTTFSPAKFASFITQYSTGQVSVEIEWELQKENSKRWNPEIQYTSNYNKSVNVDFTVDTNCVYSEPRPIGTRYLTRNL

SEQ ID NO: 15 - protein, 737 aa, Artificial Sequence
  CMRI_14 capsid polypeptide (VP1)
MAADGYLPDWLEDTLSEGIRQWWKLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSSGIGKTGQQPAKKRLNFGQTGDSESVPEPQPLGEPPATPAAVGPTTMASGGGAPMADNNEGADGVGNASGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPSTTFSPAKFASFITQYSTGQVSVEIEWELQKENSKRWNPEIQYTSNYNKSVNVDFTVDTNGVYSEPRPIGTRYLTRNL

SEQ ID NO: 16 - protein, 736 aa, Artificial Sequence
  CMRI_15 capsid polypeptide (VP1)
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSSGIGKTGQQPAKKRLNFGQTGDSESVPDPQPLGEPPATPAAVGPTTMASGGGAPMADNNEGADGVGNSSGNWHCDSQWMGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMLRTGNNFQFTYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTSVDFAVNTEGVYSEPRPIGTRYLTRPL

SEQ ID NO: 17 - protein, 737 aa, Artificial Sequence
  CMRI_16 capsid polypeptide (VP1)
MAADGYLPDWLEDTLSEGIRQWWKLKPGAPKPKANQQKQDDGRGLVLPGYKYLGPFNGLDKGEPVNAADAAALENDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPSTTFSPAKFASFITQYSTGQVSVEIEWELQKENSKRWNPEIQYTSNYNKSVNVDFTVDTNGVYSEPRPIGTRYLTRNL

SEQ ID NO: 18 - protein, 737 aa, Artificial Sequence
  CMRI_17 capsid polypeptide (VP1)
MAADGYLPDWLEDNLSEGIREWWDLKPGAPKPKANQQKQDDGRGLVLPGYKYLGPFNGLDKGEPVNEADAAALEHDKAYDRQLDSGDNPYLKYNHADAEFQERLKEDTSFGGNLGRAVFQAKKRILEPLGLVEEAAKTAPGKKRPVEPSPQRSPDSSSGIGKTGQQPAKKRLNFGQTGDSESVPDPQPLGEPPATPAAVGPTTMASGGGAPMADNNEGADGVGNSSGNWHCDSQWMGDRVITTSTRTWALPTYNNHLYKQISSASTGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMLRTGNNFQFTYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTSVDFAVNTEGVYSEPRPIGTRYLTRPL

SEQ ID NO: 19 - protein, 735 aa, Artificial Sequence
  CMRI_18 capsid polypeptide (VP1)
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNIQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - protein, 735 aa, Artificial Sequence
  CMRI_19 capsid polypeptide (VP1)
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNDNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 21 - protein, 735 aa, Artificial Sequence
  CMRI_20 capsid polypeptide (VP1)
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEQFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 22 - protein, 735 aa, Artificial Sequence
  CMRI_21 capsid polypeptide (VP1)
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTSLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 23 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGNQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 24 - protein, 735 aa, Adeno-associated virus; identical to SEQ ID NO: 5

SEQ ID NO: 25 - protein, 735 aa, Adeno-associated virus; identical to SEQ ID NO: 6

SEQ ID NO: 26 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 27 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQESLKEDTSFGGNLGRAVFQAKKRVLEPLGLVEEPVKTAPGKKRPVEHSPAEPDSSSGTGKAGQQPAKKRLNFGQTGDSDSVPDPQPLGEPPAAPTSLGSTTMASGGGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSASGTVQQSRLLFSQAGPTSMSLQAKNWLPGPCYRQQRLSKQANENNNSNFPWTAATKYHLNGRDSLVNPGPPMASHKDDEEKFFPMHGTLIFGKEGTNANNAELENVMITDEEEIRTTNPVATEQYGNVSNNLQNSNTAPSTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 28 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaagcgggccagcagcctgcaagaaaaagattgaattttggtcagactggagacgcagactccgtacctgacccccagcctctcggacagccaccagcagccccctctggtctgggatctactacaatggctacaggcagtggcgcaccagtggcagacaataacgagggtgccgatggagtgggtaattcctcaggaaattggcattgcgattcccaatggctgggcgacagagtcatcaccaccagcacccgaacctgggccctgcccacctacaacaaccacctctacaagcaaatctccagccaatcaggagcctcaaacgacaaccactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaagtgtttactgactcggagtaccagctcccgtacgtcctcggctcggcgcatcaaggatgcctcccgccgtttccagcggacgtcttcatggtcccacagtatggatacctcaccctgaacaacgggagtcaggcggtaggacgctcttcattttactgcctggagtactttccttctcagatgctgcgtactggaaacaactttcagttcagctacacttttgaagacgttcctttccacagcagctacgctcacagccagagtctggatcggctgatgaatcctctgatcgaccagtacctgtattatctgaacaagacacaaacaaatagtggaactcttcagcagtctcggctactgtttagccaagctggaccaaccaacatgtctcttcaagctaaaaactggctgcctggaccttgctacagacaacagcgtctgtcaaagcaggcaaacgacaacaacaacagcaactttccctggactgcagctacaaagtatcatctaaatggccgggactcgttagttaatccaggaccagctatggccagccacaaagacgatgaagaaaagtttttccccatgcatggaaccctgatatttggtaaacaaggaacaaatgccaacgacgcggatttggaaaatgtcatgattacagatgaagaagaaatcaggaccaccaatcccgtggctacggagcagtatgggactgtgtcaaataatttgcaaaactcaaacactggtccaactactggaactgtcaatcaccaaggagcgttacctggtatggtgtggcaggatagagacgtgtacctgcagggacccatttgggccaagattcctcacaccgatggacactttcatccttctccactgatgggaggttttgggctcaaacacccgcctcctcagatcatgatcaaaaacactcccgttccagccaatcctcccacaaacttcagttctgccaagtttgcttctttcatcacacagtattccacggggcaggtcagcgtggagatcgagtgggagctgcagaaggagaacagcaaacgctggaatcccgagatccagtacacttccaactataacaaatctgttaatgtggactttactgtggacactaatggtgtgtattcagagcctcgccccattggcaccagatacctgactcgtaatctgtaa

SEQ ID NO: 29 - DNA, 2208 bp, Adeno-associated virus
atggctgccgatggttatcttccagattggctcgaggacactctctctgaaggaataagacagtggtggaagctcaaacctggcccaccaccgccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggccaaaaagaggattcttgaacctctgggcctggttgaggagcctgttaagacggctccgggaaaaaagaggccggtagagcactctcctgcggagccagattcctcctccggaactggaaagtcgggccaacagcctgcaagaaagagattgaatttcggtcagactggagacgcagactccgtacctgacccccagcctctcggacagccaccagcagccccctctggtctgggaactaatacgatggcttcaggcagtggcgcaccaatggcagacaataacgagggcgccgatggagtgggtaattcctcaggaaattggcattgcgattcccaatggctgggcgacagagtcatcaccaccagcacccgaacctgggccctgcccacctacaacaaccacctctacaagcaaatctccagccaatcaggagcctcaaacgacaaccactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgtttccagcggacgtcttcatggtcccacagtatggatacctcaccctgaacaacgggagtcaggcggtaggacgctcttccttttactgcctggagtactttccttctcagatgctgcgtactggaaacaactttaccttcagctacacctttgaagacgttcctttccacagcagctacgctcacagccaaagtctggaccgtctcatgaatcctctcatcgaccagtacctgtattacttgagcagaacaaacacgccaagcggaaccaccacgatgtccaggcttcagttttctcaggccggagcaagtgacattcgggaccagtctagaaactggcttcctggaccctgttaccgccagcagcgagtatcaaagacagctgcggacaacaacaacagtgattactcgtggactggagctaccaagtaccacctcaatggaagagactctctggtgaatccgggcccagctatggccagccacaaggacgatgaagaaaaatattttcctcagagcggggttctcatctttggaaaacaagactcgggaaaaactaatgtggacattgaaaaggttatgattacagacgaagaggaaatcaggaccaccaatcccgtggctacggagcagtatggttctgtatctaccaacctccagagcggcaacacacaagcagctacctcagatgtcaacacacaaggcgttcttcctggcatggtctggcaggacagagacgtgtacctgcaggggcccatttgggcaaagattccccacacggatggacactttcacccttctcctcttatgggaggatttggactgaagcaccctcccccgcagattctcatcaagaataccccggtacctgcgaatccttcgaccaccttcagtgcggcaaagtttgcttctttcatcacacagtactccacggggcaggtcagcgtggagatagagtgggagctgcagaaggagaacagcaaacgctggaaccccgagatccagtatacttccaactacaacaaatctgttaatgtggactttactgtggacactaatggtgtgtattcagagcctcgccccattggcaccagatacctgactcgtaatctgtaa

SEQ ID NO: 30 - DNA, 2205 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaaaggagagccggtcaacgaggcagacgccgcggccctcgagcacgacaaggcctacgaccggcagctcgacagcggagacaacccgtacctcaaatacaaccacgccgacgcggagtttcaggagcgtcttaaagaagatacgtcttttgggggcaacctcggacgagcagtcttccaggcgaaaaagagggttcttgaacctctgggcctggttgaggagcctgttaagacggctccgggaaaaaagaggccggtagagcactctcctgcggagccagactcctcctcgggaaccggaaaggcgggccagcagcctgcaagaaaaagactgaattttggtcagactggagacgcagactccgtaccagaccctcaacctctcggagaaccaccagcagcccccacaagtttgggatctactacaatggcttcaggcggtggcgcaccagtggcagacaataacgagggtgccgatggagtgggtaattcctcaggaaattggcattgcgattcccaatggctgggcgacagagtcatcaccaccagcacccgcacctgggctttacccacctacaacaaccatctgtacaagcaaatctccagccaatcaggagccagcaacgacaatcactactttggctacagcaccccctgggggtattttgacttcaacagattccactgccacttttcaccacgtgactggcaaagacttatcaacaacaactggggattccggcccaagagactcaacttcaagctctttaacattcaagtcaaagaggtcacgcagaatgacggtacgacgacgattgccaataaccttaccagcacggttcaggtgtttactgactcggaataccagctcccgtacgtcctcggctcggcgcatcaaggatgcctcccgccgtttccagcggacgtcttcatggtcccacagtatggatacctcaccctgaacaacgggagtcaggcggtaggacgctcttccttttactgtctggagtactttccttctcagatgctgcgtactggaaacaactttcagttcagctacacctttgaagacgtgcctttccacagcagctacgctcacagccagagtctggatcggctgatgaatcctctcatcgatcagtacctgtattatctgaacaggacacaaacagccagtggaactcagcagtctcggctactgtttagccaagctggacccaccagtatgtctcttcaagctaaaaattggctgcctggaccttgctacagacagcaacgtctgtcaaagcaggcaaacgacaacaacaacagcaactttccctggactgcggctacaaagtatcatctaaatggccgggactcgttagttaatccaggaccagctatggccagtcacaaggatgacgaagaaaaatttttccccatgcacggaaccctgatatttggtaaagaaggaacaaatgctaacaacgcggaattggaaaatgtcatgattacagatgaagaagaaatccgcaccacaaatcctgtagctacggagcagtacggatatgtgtcaaataatttgcaaaactcaaatactgctgcaactactgaaactgtcaatcatcaaggagcgttacctggtatggtgtggcaggacagagacgtgtacctgcagggacccatttgggccaagattcctcacacggatggacactttcacccttctccactgatgggaggttttggactcaaacacccgcctcctcagatcatgatcaaaaacactcccgttccagctaatcctcccacaaacttcagtgcagcaaagtttgcttccttcatcacacagtattccacggggcaggtcagcgtggaaattgagtgggagctgcagaaagagaacagcaaacgctggaaccccgagatccagtacacttccaactacaacaaatctgttaatgtggactttactgtggacactaatggtgtgtattcagagcctcgccccattggcaccagatacctgactcgtaatctgtaa

SEQ ID NO: 31 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggagcctgttaagacggctccgggaaaaaagaggccggtagagcactctcctgtggagccagactcctcctcgggaaccggaaaagcgggccagcagcctgcgagaaagagattgaatttcggtcagactggagacgcagactccgtacctgacccccagcctctcggacagccaccagcagccccctctggtctgggaactaatacgatggctacaggcagtggcgcaccaatggcagacaataacgagggtgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagcatcaaacgacaaccactactttggctacagcaccccttgggggtattttgacttcaacagattccactgccacttctccccacgtgactggcaaagactcatcaacaacaattggggattccggcccaaaagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttcattttactgcctggagtactttccttctcagatgctgcgtaccggaaacaactttaccttcagctacacctttgaggacgttcctttccacagcagctacgctcacagccagagtttggaccggctgatgaatcctctgatcgaccagtatctgtattacttgagcaaaacaaacgctccaagcggaaccaccacgatgtccaggcttcagttttctcaggccggagcaagtgacattcgggaccagtctagaaactggcttcctggaccctgttaccgccagcagcgagtatcaaagacagctgcggacaacaacaacagtgattactcgtggactggagctaccaagtaccacctcaatggaagagactctctggtgaatccgggcacagccatggccagccacaaggacgatgaagaaaagtattttcctcagagcggggttctcatcttcggaaaacaagactcgggaaaaactaatgtggacattgaaaaggtcatgattacagacgaagaggaaatcaggaccaccaatcccgtggctacagagcagtatggttctgtatctaccaacctccaaagcggcaacacacaagcagctacctcagatgtcaacacacaaggcgttcttccaggcatggtctggcaggacagagacgtgtacctgcagggacccatctgggcaaagattccacacacggacggacattttcacccttctcctcttatgggaggatttggactgaagcaccctcccccgcagattctcatcaagaataccccggtacctgcgaatccttctaccaccttcagtgcggcaaagtttgcttccttcatcacacagtactccacggggcaggtcagcgtggagatcgagtgggagctgcagaaagagaacagcaaacgctggaatcccgagatccagtacacttcaaactacaacaagtctgttaatgtggactttactgtggatactaatggtgtgtattcagagcctcgccccattggcaccagatacctgactcgtaatctgtaa

SEQ ID NO: 32 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgataaggcctacgaccggcagctcgacagcggagacaacccgtacctcaagtacaaccacgccgacgcggagtttcaggagcgccttaaagaagatacgtcttttgggggcaacctcggacgagcagtcttccaggccaaaaagagggttcttgaacctctgggcctggttgaggagcctgttaagacggctccgggaaaaaagaggccggtagagcactctcctgcggagccagactcctcttccggaaccggaaaagcgggccagcagcctgcaaaaaagagactgaatttcggtcagactggagactcagactccgtaccagaccctcaacctctcggagaaccaccagcagcccccacaagtttgggatctactacaatggcttcaggcggtggcgcaccagtggcagacaataacgagggtgccgatggagtgggtaattcctcgggaaattggcattgcgattcccaatggctgggcgacagagtcatcaccaccagcacccgaacctgggccctgcccacctacaacaaccatctgtacaaacagatttctagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaacttccaattcagctacacctttgaggacgttcctttccacagcagctatgctcatagccagagtctggatcggctaatgaatcctctgatcgaccagtacctgtattatctgaacaagacacaatcagccagtggaactgttcagcagtctcggctactgtttagccaagctggacccacaagcatgtctcttcaagctaaaaactggctgcctggaccttgctacagacagcaacgtctgtcaaagcaggcaaacgaaaacaacaacagcaactttccctggactgcggctaccaagtatcatctaaatggccgggactcgttagttaatccaggaccgcctatggccagtcacaaggatgacgaagaaaagtttttccccatgcacggaaccctgatatttggtaaagaaggaacaaatgctaacaacgcggaattggaaaacgtcatgattacagatgaagaagaaatccgcaccacaaatcctgtagctacggagcaatacggcaatgtgtcaaataatttgcaaaactcaaatactgctccaagtactggaactgtcaatcaccaaggagcgttacctggtatggtgtggcaagatagagacgtgtacctgcagggacccatttgggccaagattcctcacaccgatggacattttcatccttctccgctgatgggaggttttggactcaaacacccgcctcctcagatcatgattaaaaacactcccgttccagccaatcctcctacaaacttcagtgcggcaaagtttgcttccttcatcacacagtattccacggggcaggtcagcgtggagatcgagtgggagctgcaaaaggagaacagcaaacgctggaatcccgagatccagtacacttccaactacaacaagtctgttaatgtggactttactgtggacactaatggcgtgtattcagagcctcgccccattggcaccagatacctgactcgtaatctgtaa

SEQ ID NO: 33 - DNA, 2208 bp, Adeno-associated virus
atggctgccgatggttatcttccagattggctcgaggacactctctctgaaggaataagacagtggtggaagctcaaacctggcccaccaccaccaaagcccgcggagcggcataaggacaacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgccttaaagaagatacgtcttttgggggcaacctcggacgagcagtcttccaggccaaaaagagggttcttgaacctctgggcctggttgaggagcctgttaagacggctccgggaaaaaagaggccggtagagcactctcctgtggagccagactcctcctcgggaaccggaaaagcgggccagcagcctgcaaaaaagagactgaatttcggtcagactggagacgcagactccgtacctgacccccaacctctcggacaaccaccagcagccccctctggtctgggatctactacaatggctacaggcagtggcgcaccagtggcagacaataacgagggtgccgatggagtgggtaattcctcgggaaattggcattgcgattcccaatggctgggcgacagagtcatcaccaccagcacccgaacctgggccctgcccacctacaacaaccatctgtacaaacagatttccagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgttttcatggtgccacagtatggatacctcaccctgaacaacgggagtcaggcagtaggacgctcttcattttactgcctggagtactttccttctcagatgctgcgtactggaaacaactttcagttcagctacacctttgaagacgttcctttccacagcagctacgctcacagccagagtttggatcggctgatgaatcctctgatcgaccagtacctgtattatctgaacaagacacaatcaaatagtggaactcttcagcagtctcggctactgtttagccaagctggacccaccagcatgtctcttcaagctaaaaattggctgcctggaccttgctacagacaacagcgtctgtcaaagcaggcaaacgacaacaacaacagcaactttccctggactgcggctacaaagtatcatctaaatggccgggactcgttggttaatccaggaccagctatggccagccacaaagacgatgaagaaaagtttttccccatgcatggaaccctaatatttggtaaacaaggaacaaatgctaacgacgcggatttggacaatgtcatgattacagatgaagaagaaatccgcaccaccaatcccgtggctacggagcagtacggatatgtgtcaaataatttgcaaaactcaaatactggtccaactactggaactgtcaatcaccaaggagcgttacctggtatggtgtggcaggatcgagacgtgtacctgcagggacccatttgggccaagattcctcacaccgatggacactttcatccttctccactgatgggaggttttggactcaaacacccacctcctcagatcatgattaaaaacactcccgttccagccaatcctcccacaaacttcagttctgccaagtttgcttctttcatcacacagtattccacgggacaggtcagcgtggaaatcgagtgggagctgcagaaggagaacagcaaacgctggaatcccgaaattcagtacacttccaactacaacaaatctgttaatgtggactttactgtggacactaatggtgtgtattcagagcctcgccccattggcaccagatacctgactcgtaatctataa

SEQ ID NO: 34 - DNA, 2208 bp, Adeno-associated virus
atggctgccgatggttatcttccagattggctcgaggacaccctctctgaaggaataagacagtggtggaagctcaaacctggcccaccaccaccaaagcccgcagagcggcataaggacgacagcaggggtcttgtgcttcctgggtacaagtacctcggacccttcaacggactcgacaaaggagagccggtcaacgaggcagacgccgcggccctcgagcacgacaaggcctacgaccggcagctcaacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggagcctgttaaaacggctccgggaaaaaagaggccggtagagcactctcctgcggagccagactcctcctcgggaaccggaaaggcgggccagcagcctgctagaaaaagactgaatttcggtcagactggagacgcagactccgtaccagaccctcaacctctcggagaaccaccagcagcccccacaagtttgggatctactacaatggcttcaggcggtggcgcaccagtggcagacaataacgagggtgccgatggagtgggtaattcctcgggaaattggcattgcgattcccaatggctgggggacagagttatcaccaccagcacccgaacctgggccctgcccacctacaacaaccacctctacaagcaaatttccagccaatcaggagccagcaacgacaaccactactttggctacagcaccccctgggggtattttgacttcaacagattccactgccacttttccccacgtgactggcaaagactcatcaacaacaactggggattccgacccaaaagactcaacttcaagctctttaacattcaagtcaaggaggtcacgcagaatgacggtacgacgacgattgccaataaccttaccagcacggttcaggtgtttactgactcggaataccagctcccgtacgtcctcggctcggcgcatcaaggatgcctcccgccgtttccagcggacgtcttcatggtcccacagtatggatacctcaccctgaacaacgggagtcaggcggtaggacgctcttccttttactgcctggagtactttccttctcagatgttgcgtactggaaacaactttcagttcagctacacctttgaagacgttcctttccacagcagctacgctcacagccagagtctggatcggctgatgaatcctctaatcgaccagtacctgtattatctgaataagacacaaaactccagtggaactgttcaacagtctcggctactgtttagtcaagctggacccaccagtatgtctcttcaagctaaaaactggctgcctggaccttgctacagacagcagcgtctgtcaaagcaggcaagcgacaacaacaacagcaactttccctggactgcggccacaaagtatcatctaaatggccgggactcgttggttaatccaggaccagctatggccagtcacaaggatgacgaagaaaagtttttccccatgcatggaaccctaatatttggtaaagaaggaacaactgctaacaacgcggatttggaacatgtcatgattacagatgaagaagaaatcaggaccaccaatcccgtggctacggagcagtacggaaatgtgtcaaataatttgcaaaactcaaatactggtccaactactgaaaatgtcaataaccaaggagcgttacctggtatggtgtggcaggatcgagacgtgtacctgcagggacccatttgggccaagattcctcacaccgatggacactttcatccttctccactgatgggaggttttggactcaaacatcctcccccgcagatcatgatcaaaaacactcccgttccagccaatcctcccacaaacttcagtgcggcaaagtttgcttccttcatcacacagtactccactgggcaggtcagcgtggagatcgagtgggagctgcagaaggagaacagcaaacgctggaaccccgagatccagtacacttccaactacaacaaatctgttaatgtggactttactgtggacactaatggtgtgtattcagagcctcgccccattggcaccagatacctgactcgtaatctgtaa

SEQ ID NO: 35 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggagcctgttaaaacggctccgggaaaaaagaggccggtagagcactctcctgcggaaccagactcctcctcgggaaccggaaaggcgggccagcagcctgctagaaaaagactgaatttcggtcagactggagacgcagactccgtaccagaccctcaacctctcggagaaccaccagcagcccccacaagtgtgggatctactacaatggcttcaggcggtggcgcaccagtggcagacaataacgagggtgccgatggagtgggtaattcctcaggaaattggcattgcgattcccaatggctgggcgacagagtcattaccaccagcacccgaacctgggccctgcccacctacaacaaccatctctacaagcaaatttccagccaatcaggagccagcaacgacaatcactactttggctacagcaccccctgggggtattttgacttcaacagattccactgccacttttcaccacgtgactggcaaagacttatcaacaacaactggggattccggcccaagagactcaacttcaagctctttaacattcaagtcaaagaggtcacgcagaatgacggtacgacgacgattgccaataaccttaccagcacggttcaggtgtttactgactcggaataccagctcccgtacgtcctcggctcggcgcatcaaggatgcctcccgccgtttccagcggacgtcttcatggtcccacagtatggatacctcaccctgaacaacgggagtcaggcggtaggacgctcttccttttactgcctggagtactttccttctcagatgctgcgtacaggaaacaactttcagttcagctacacctttgaagacgtgcctttccacagcagctacgctcacagccagagtctggatcggctgatgaatcctctgatcgaccagtacctgtattatctgaataagacacaaaactccagtggaactgttcagcagtctcggctactgtttagccaagctggacccaccagcatgtctcttcaagctaaaaattggctgcctggaccttgctacagacagcaacgtctgtcaaagcaggcaagcgacaacaacaacagtaactttccctggactgcggccacaaagtatcatctaaatggccgggactcgttggttaatccaggaccagctatggccagtcacaaggatgacgaagaaaagtttttccccatgcacggaaccctaatatttggtaaagaaggaacaactgctaacaacgcggatttggaacatgtcatgattacagatgaagaagaaatcaggaccaccaatccagtggctacggagcagtacggaaatgtatcaaataatttgcaaaactcaaatactggtccaactactgaaaatgtcaataaccaaggagcgttacctggtatggtgtggcaggatcgagacgtgtacctgcagggacccatttgggccaaaattcctcacaccgacggacactttcatccttctccactgatgggaggttttggactcaaacacccacctcctcagatcatgatcaaaaacactccagttccagccaatcctcctacaaacttcagtgcggcaaagtttgcttccttcatcacacagtactccacggggcaggtcagcgtggagatcgagtgggagctgcagaaggagaacagcaaacgctggaaccccgagatccagtacacttccaactacaacaaatctgttaatgtggactttactgtggacactaatggtgtgtattcagagcctcgccccattggcaccagatacctgactcgtaatttgtaa

SEQ ID NO: 36 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgctgacgcggagtttcaggagcgccttaaaaaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaagcgggccagcagcctgcaagaaaaagattgaattttggtcagactggagacgcagactccgtacctgacccccagcctctcggacagccaccagcagccccctctggtctgggatctactacaatggctacaggcagtggcgcaccagtggcagacaataacgagggtgccgatggagtgggtaattcctcaggaaattggcattgcgattcccaatggctgggcgacagagtcatcaccaccagcacccgaacctgggctctgcccacctacaacaaccacctctacaagcaaatctccagccaatcaggagccagcaacgacaaccactactttggctacagcaccccctgggggtattttgacttcaacagattccattgccacttttcaccacgtgactggcaaagactcatcaacaacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttccttttactgcctggagtactttccttctcagatgctgcgtactggaaacaactttcagttcagctacacctttgaagacgttcctttccacagcagctacgctcacagccagagtctggatcggctgatgaatcctctgatcgaccagtacctgtattatctgaacaagacacaatcaaatagtggaactcttcagcagtctcggctactgtttagccaagctggacccaccagcatgtctcttcaagctaaaaactggctgcctggaccttgctacagacagcagcgtttgtcaaagcaggcaaacgacaacaacaacagcaactttccctggactgcagctacaaagtatcatctaaatggccgggactcgttggttaatccaggaccagctatggccagtcacaaggatgacgaagaaaagtttttccccatgcatggaaccctcatatttggtaaacaaggaacaaatgccaacgacgcggatttggacaatgtcatgattacagatgaagaagaaatccgcaccaccaatcccgtggctacggagcagtacggatatgtgtcaaataatttgcaaaactcaaatactggtccaactactggaactgtcaatcaccaaggagcgttacctggtatggtgtggcaggatcgagacgtgtacctgcagggacccatttgggccaagattcctcacaccgatggacactttcatccttctccactgatgggaggttttggactcaaacacccacctcctcagatcatgatcaaaaacactcccgttccagccaatcctcctacaaacttcagttctgccaagtttgcttctttcatcacacagtattccacggggcaggtcagcgtggagatcgagtgggagctacagaaggagaacagcaaacgctggaatcccgaaattcagtacacttccaactacaacaaatctgttaatgtggactttactgtggacactaatggtgtgtattcagagcctcgccccattggcaccagatacctgactcgtaatctgtaa

SEQ ID NO: 37 - DNA, 2208 bp, Artificial Sequence
  CMRI_10 capsid polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggccaaaaagagggttcttgaacctttgggcctggttgaggaacctgttaagacggctccgggaaaaaagaggccggtagagcactctcctgcggagccagactcctcttccggaaccggaaaagcgggccagcagcctgcaaaaaagagactgaatttcggtcagactggagactcagactccgtaccagaccctcaacctctcggagaaccaccagcagcccccacaagtttgggatctactacaatggcttcaggcggtggcgcaccagtggcagacaataacgagggtgccgatggagtgggtaattcctcgggaaattggcattgcgattcccaatggctgggcgacagagtcatcaccaccagcacccgaacctgggccctgcccacctacaacaaccatctgtacaaacagatttccagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaacttccaattcagctacacctttgaggacgttcctttccacagcagctatgctcacagccagagtctggatcggctaatgaatcctctgatcgaccagtacctgtattatctgaacaagacacaatcagccagtggaactgttcagcagtctcggctactgtttagccaagctggacccacaagcatgtctcttcaagctaaaaactggctgcctggaccttgctacagacagcaacgtctgtcaaagcaggcaaacgaaaacaacaacagcaactttccctggactgcggctaccaagtatcatctaaatggccgggactcgttagttaatccaggaccgcctatggccagtcacaaggatgacgaagaaaagtttttccccatgcacggaactctgatatttggtaaagaaggaacaaatgctaacaacgcggaattggaaaacgtcatgattacagatgaagaagaaatccgcaccacaaatcctgtagctacggagcaatacggcaatgtgtcaaataatttgcaaaactcaaatactgctccaagtactggaactgtcaatcaccaaggagcgttacctggtatggtgtggcaagatagagacgtgtacctgcagggacccatttgggccaagattcctcacaccgatggacattttcatccttctccgctgatgggaggttttggactcaaacacccgcctcctcagatcatgattaaaaacactcccgttccagccaatcctcctacaaacttcagtgcggcaaagtttgcttccttcatcacacagtattccacggggcaggtcagcgtggagatcgagtgggagctgcaaaaggagaacagcaaacgctggaaccccgagatccagtacacttccaactacaacaagtctgttaatgtggactttactgtggacactaatggcgtgtattcagagcctcgccccattggcaccagatacctgactcgtaatctgtaa

SEQ ID NO: 38 - DNA, 2214 bp, Artificial Sequence
  CMRI_11 capsid polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaaccttggcagagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgctaaaaagagactcaattttggtcagactggcgactcagagtcagtcccagaccctcaacctctcggagaacctccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaatcacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccgtcgcaaatgctaaggactggaaataacttccaattcagctataccttcgaggatgtaccttttcacagcagctacgctcacagccagagtttggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattccacacacggacggacattttcacccctctcccctcatgggtggattcggacttaaacaccctcctccacagattctcatcaagaacaccccggtacctgcgaatccttcgaccaccttcagcccggccaagtttgcttcatttatcactcagtactccactggacaggtcagcgtggaaattgagtgggagctacagaaagaaaacagcaagcgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 39 - DNA, 2214 bp, Artificial Sequence
  CMRI_12 capsid polynucleotide
atggctgccgatggttatcttccagattggctcgaggacaacctctctgaaggaataagacagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaaccttggcagagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgctaaaaagagactcaattttggtcagactggcgactcagagtcagtcccagaccctcaacctctcggagaacctccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaatcacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccgtcgcaaatgctaaggactggaaataacttccaattcagctataccttcgaggatgtaccttttcacagcagctacgctcacagccagagtttggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattccacacacggacggacattttcacccctctcccctcatgggtggattcggacttaaacaccctcctccacagattctcatcaagaacaccccggtacctgcgaatccttcgaccaccttcagcccggccaagtttgcttcatttatcactcagtactccactggacaggtcagcgtggaaattgagtgggagctacagaaagaaaacagcaagcgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 40 - DNA, 2214 bp, Artificial Sequence
  CMRI_13 capsid polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaaccttggcagagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgctaaaaagagactcaattttggtcagactggcgactcagagtcagtcccagaccctcaacctctcggagaacctccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaatcacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccgtcgcaaatgctaaggactggaaataacttccaattcagctataccttcgaggatgtaccttttcacagcagctacgctcacagccagagtttggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattccacacacggacggacattttcacccctctcccctcatgggtggattcggacttaaacaccctcctccacagattctcatcaagaacaccccggtacctgcgaatccttcgaccaccttcagcccggccaagtttgcttcatttatcactcagtactccactggacaggtcagcgtggaaattgagtgggagctacagaaagaaaacagcaagcgctggaatcccgaaattcagtacacttccaactacaacaagtctgttaatgtggactttactgtggacactaattgcgtgtattcagagcctcgccccattggcaccagatacctgactcgtaatctgtaa

SEQ ID NO: 41 - DNA, 2214 bp, Artificial Sequence
  CMRI_14 capsid polynucleotide
atggctgccgatggttatcttccagattggctcgaggacactctctctgaaggaataagacagtggtggaagctcaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctcctcgggcatcggcaagacaggccagcagcccgctaaaaagagactcaattttggtcagactggcgactcagagtcagtccccgaaccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattcccaatggctgggggacagagtcatcaccaccagcacccgaacctgggccctgcccacctacaacaatcacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccgtcgcaaatgctaaggactggaaataacttccaattcagctataccttcgaggatgtaccttttcacagcagctacgctcacagccagagtttggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattccacacacggacggacattttcacccctctcccctcatgggtggattcggacttaaacaccctcctccacagattctcatcaagaacaccccggtacctgcgaatccttcgaccaccttcagcccggccaagtttgcttcatttatcactcagtactccactggacaggtcagcgtggaaattgagtgggagctacagaaagaaaacagcaagcgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 42 - DNA, 2211 bp, Artificial Sequence
  CMRI_15 capsid polynucleotide
atggctgccgatggttatcttccagattggctcgaggacaacctttctgaaggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaaacgtccggtagagccatcaccccagcgttctccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgagggcgccgacggagtgggtaattcctcaggaaattggcattgcgattcccaatggatgggcgacagagtcatcaccaccagcaccagaacctgggccctgcccacttacaacaaccatctctacaagcaaatctccagccaatcaggagcttcaaacgacaaccactactttggctacagcaccccctgggggtattttgacttcaacagattccactgccacttctcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctgactctcaacaatggcagtcagtctgtgggacgttcctccttctactgcctggaatattttccttcgcagatgctgagaaccggcaacaacttccagtttacttacaccttcgaggacgtgcctttccacagcagctacgcccacagccagagcctggaccggctgatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtctggcagaaccgagacgtgtacctgcagggtcccatctgggccaagattcctcacacggacggcaactttcacccgtctcctctgatgggcggctttggacttaaacacccgcctccacagatcctgatcaagaacacgccggtacctgcggatcctccaacaacgttcagccaggcgaaattggcttccttcatcacgcagtacagcaccggacaggtcagcgtggaaatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtcccctgtaa

SEQ ID NO: 43 - DNA, 2214 bp, Artificial Sequence
  CMRI_16 capsid polynucleotide
atggctgccgatggttatcttccagattggctcgaggacactctctctgaaggaataagacagtggtggaagctcaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagaacgacaaggcctacgaccagcagctcaaagcgggtgacaatccgtacctgcggtataaccacgccgacgccgagtttcaggagcgtctgcaagaagatacgtcttttgggggcaaccttggcagagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgctaaaaagagactcaattttggtcagactggcgactcagagtcagtcccagaccctcaacctctcggagaacctccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaatcacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccgtcgcaaatgctaaggactggaaataacttccaattcagctataccttcgaggatgtaccttttcacagcagctacgctcacagccagagtttggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattccacacacggacggacattttcacccctctcccctcatgggtggattcggacttaaacaccctcctccacagattctcatcaagaacaccccggtacctgcgaatccttcgaccaccttcagcccggccaagtttgcttcatttatcactcagtactccactggacaggtcagcgtggaaattgagtgggagctacagaaagaaaacagcaagcgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 44 - DNA, 2214 bp, Artificial Sequence
  CMRI_17 capsid polynucleotide
atggctgccgatggttatcttccagattggctcgaggacaacctctctgagggcattcgcgagtggtgggacctgaaacctggagccccgaaacccaaagccaaccagcagaagcaggacgacggccggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggcagagcagtcttccaggccaaaaagaggatccttgagcctcttggtctggttgaggaagcggctaagacggctcctggaaagaaacgtccggtagagccatcaccccagcgttctccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgagggcgccgacggagtgggtaattcctcaggaaattggcattgcgattcccaatggatgggcgacagagtcatcaccaccagcacccgaacctgggccctgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctgactctcaacaatggcagtcagtctgtgggacgttcctccttctactgcctggaatattttccttcgcagatgctgagaaccggcaacaacttccagtttacttacaccttcgaggacgtgcctttccacagcagctacgcccacagccagagcctggaccggctgatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtctggcagaaccgagacgtgtacctgcagggtcccatctgggccaagattcctcacacggacggcaactttcacccgtctcctctgatgggcggctttggacttaaacacccgcctccacagatcctgatcaagaacacgccggtacctgcggatcctccaacaacgttcagccaggcgaaattggcttccttcatcacgcagtacagcaccggacaggtcagcgtggaaatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtcccctgtaa

SEQ ID NO: 45 - DNA, 2208 bp, Artificial Sequence
  CMRI_18 capsid polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacatacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 46 - DNA, 2208 bp, Artificial Sequence
  CMRI_19 capsid polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacgacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 47 - DNA, 2208 bp, Artificial Sequence
  CMRI_20 capsid polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaacagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 48 - DNA, 2208 bp, Artificial Sequence
  CMRI_21 capsid polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccagcctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 49 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggagcctgttaaaacggctccgggaaaaaagagaccggtagagcactctcctgcggagccagactcctcctcgggaaccggaaaggcgggcaaccagcctgcaagaaaaagattgaatttcggtcagactggagacgcagactccgtacctgacccccagcctctcggacagccaccagcagccccctctggtttgggatctactacaatggctacaggcagtggcgcaccagtggcagacaataacgagggtgccgatggagtgggtaattcctcaggaaattggcattgcgattcccaatggctgggcgacagagtcatcaccaccagcacccgaacctgggccctgcccacctacaacaaccacctctacaagcaaatctccagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttccttttactgcctggagtactttccttctcagatgctgcgtactggaaacaactttcagttcagctacacctttgaagacgttcctttccacagcagctacgctcacagccagagtctggatcggctgatgaatcctctgatcgaccagtacctgtattatctgaacaaaacacaatcaaatagtggaactcttcagcagtctcggctactgtttagtcaagctggacccaccagcatgtctcttcaagctaaaaactggctgcctggaccttgctacagacagcagcgtctgtcaaagcaggcaaacgacaacaacaacagcaactttccctggactgcggctacaaagtatcatctaaatggccgggactcgttggttaatccaggaccagctatggccagccacaaagacgatgaagaaaagtttttccccatgcatggaaccctgatatttggtaaacaaggaacaaatgccaacgacgcggatttggacaatgttatgattacagatgaagaagaaatccgcaccaccaatcccgtggctacggagcagtacggatatgtgtcaaataatttgcaaaactcaaacactggtccaactacaggaactgtcaatcaccaaggagcgttacctggtatggtgtggcaggatcgagacgtgtacctgcagggacccatttgggccaagattcctcacacggacggacattttcatccttctccactgatgggaggttttgggctcaaacacccgcctcctcagatcatgatcaaaaacactcccgttccagccaatcctcctacaaacttcagttctgccaagtttgcttctttcatcacacagtattccacggggcaggtcagcgtggagatcgagtgggagctgcagaaggagaacagcaaacgctggaatcccgaaattcagtatacttccaactacaacaaatctgttaatgtggactttactgtggacactaatggcgtgtattcagagcctcgacccattggcaccagatacctgactcgtaatctgtaa

SEQ ID NO: 50 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagccggtcaacgaggcagacgccgcggccctcgagcacgacaaggcctacgaccggcagctcgacagcggagacaacccgtacctcaagtacaaccacgccgacgcggagtttcaggagcgccttaaagaagatacgtcttttgggggaaacctcggacgagcagtcttccaggcgaaaaagagggttcttgaacctctgggcctggttgaggagcctgttaaaacggctccgggaaaaaagaggccggtagagcactctcctgtggagccagactcctcctcgggaaccggaaaagcgggccagcagcctgcgagaaagagattgaatttcggtcagactggagacgcagactccgtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagcatcgaacgacaaccactactttggctacagcaccccctgggggtattttgacttcaacagattccactgccatttctccccacgtgactggcaaagactcatcaacaacaattggggattccgacccaagagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgtttccagcggacgtcttcatggtcccacagtatggatacctcaccctgaacaacgggagtcaggcggtaggacgctcttccttttactgcctggagtactttccttctcagatgctgcgtaccggaaacaactttaccttcagctacacctttgaggacgttcctttccacagcagctacgctcacagccagagtttggaccggctgatgaatcctctgatcgaccagtatctgtattacttgagcaaaacaaacgctccaagcggaaccaccacgatgtccaggcttcagttttctcaggcaggagcaagtgacattcgggaccagtctaggaactggcttcctggaccctgttaccgccagcagcgagtatcaaagacagctgcggacaacaacaacagtgattactcgtggactggagctaccaagtaccacctcaatggaagagactctctggtgaatccgggcacagccatggccagccacaaggacgatgaagaaaagtattttcctcagagcggggttctcatcttcggaaaacaagactcgggaaaaactaatgtggacattgaaaaggtcatgattacagacgaagaggaaatcaggaccaccaatcccgtggctacggagcagtatggttctgtatctaccaacctccaaagcggcaacacacaagcagctacctcagatgtcaacacacaaggcgttcttccaggcatggtctggcaggacagagacgtgtacctgcagggacccatctgggcaaagattccacacacggacggacattttcacccttctcctcttatgggaggatttggactgaagcacccccccccgcagattctcatcaagaataccccggtacctgcgaatccttctaccaccttcagcgcggcaaagtttgcttccttcatcacacagtactccacggggcaggtcagcgtggagatcgagtgggagctgcagaaagagaacagcaaacgctggaaccccgagatccagtacacttccaactacaacaagtctgttaatgtggactttactgtggacactaatggtgtgtattcagagcctcgccccattggcaccagatacctgactcgtaatctgtaa

SEQ ID NO: 51 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgataaggcctacgaccggcagctcgacagcggagacaacccgtacctcaagtacaaccacgccgacgcggagtttcaggagcgtcttaaagaagatacgtcttttgggggcaacctcggacgagcagtcttccaggccaaaaagagggttcttgaacctctgggcctggttgaggagcctgttaagacggctccgggaaaaaagaggccggtagagcactctcctgcggagccagactcctcttccggaaccggaaaagcgggccagcagcctgcaaaaaagagactgaatttcggtcagactggagactcagactccgtaccagaccctcaacctctcggagaaccaccagcagcccccacaagtttgggatctactacaatggcttcaggcggtggcgcaccagtggcagacaataacgagggtgccgatggagtgggtaattcctcgggaaattggcattgcgattcccaatggctgggcgacagagtcatcaccaccagcacccgaacctgggccctgcccacctacaacaaccatctgtacaaacagatttctagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaacttccaattcagctacacctttgaggacgttcctttccacagcagctatgctcacagccagagtctggatcggctaatgaatcctctgatcgaccagtacctgtattatctgaacaagacacaatcagccagtggaactgttcagcagtctcggctactgtttagccaagctggacccacaagcatgtctcttcaagctaaaaactggctgcctggaccttgctacagacagcaacgtctgtcaaagcaggcaaacgaaaacaacaacagcaactttccctggactgcggctaccaagtatcatctaaatggccgggactcgttagttaatccaggaccgcctatggccagtcacaaggatgacgaagaaaagtttttccccatgcacggaaccctgatatttggtaaagaaggaacaaatgctaacaacgcggaattggaaaacgtcatgattacagatgaagaagaaatccgcaccacaaatcctgtagctacggagcaatacggcaatgtgtcaaataatttgcaaaactcaaatactgctccaagtactggaactgtcaatcaccaaggagcgttacctggtatggtgtggcaagatagagacgtgtacctgcagggacccatttgggccaagattcctcacaccgatggacattttcatccttctccgctgatgggaggttttggactcaaacacccgcctcctcagatcatgattaaaaacactcccgttccagccaatcctcctacaaacttcagtgcggcaaagtttgcttccttcatcacacagtattccacggggcaggtcagcgtggagatcgagtgggagctgcaaaaggagaacagcaaacgctggaatcccgagatccagtacacttccaactacaacaagtctgttaatgtggactttactgtggacactaatggcgtgtattcagagcctcgccccattggcaccagatacctgactcgtaatctgtaa

SEQ ID NO: 52 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggaacgccttaaagaagatacgtcttttgggggcaacctcggacgagcagtcttccaggcgaaaaagagggttcttgaacctctgggcctggttgaggagcctgttaagacggctccgggaaaaaagaggccggtagagcactctcctgcggagccagactcctcctcgggaaccggaaaagcgggccagcagcctgcaagaaagagattgaattttggtcagactggagacgcagactccgtacctgacccccagcctctcggacagccaccagcagccccctctggtctgggatctactacaatggctacaggcagtggcgcaccagtggcagacaataacgagggtgccgatggagtgggtaattcctcaggaaattggcattgcgattcccaatggctgggcgacagagtcatcaccaccagcacccgaacctgggctctgcccacctacaacaaccacctctacaagcaaatctccagccaatcaggagcctcaaacgacaaccactactttggctacagcaccccctgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtcccacagtatggatacctcaccctgaacaacgggagtcaggcggtaggacgctcttccttttactgcctggagtactttccttctcagatgctgcgtactggaaacaactttcagttcagctacacttttgaggacgttcctttccacagcagctacgctcacagccagagtctggatcggctgatgaatcctctgatcgaccagtacctgtattatctgaacaagacacaatcaaatagtggaactcttcagcagtctcgactactgtttagtcaagctggacccaccagcatgtctcttcaagctaaaaactggctgcctggaccttgctacagacagcagcgtctgtcaaagcaggcaaacgacaacaacaacagcaactttccctggactgcggctaccaagtatcatctaaatggccgggactcgttggttaatccaggaccagctatggccagccacaaagacgatgaagaaaagtttttccccatgcatggaaccctgatatttggtaaacaaggaacaaatgctaacgacgcggatttggacaatgtcatgattacagatgaagaagaaatccgcaccaccaatcccgtggctacggagcagtacggatatgtgtcaaataatttgcaaaactcaaatactggtccaactactggaactgtcaatcaccaaggagcgttacctggtatggtgtggcaagatcgagacgtgtacctgcagggacccatttgggccaagattcctcacaccgatggacactttcatccttctccattgatgggaggttttggactcaaacacccgcctcctcagatcatgatcaaaaacactcccgttccagccaatcctcccacaaacttcagttctgccaagtttgcttctttcatcacccagtattccacgggacaggtcagcgtggagatcgagtgggagctgcagaaagagaacagcaaacgctggaatcccgagatccagtacacttccaactataacaaatctgttaatgtggactttactgtggacactaatggtgtgtattcagagcctcgccccattggcaccagatacctgactcgtaatctgtaa

SEQ ID NO: 53 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagagtcttaaagaagatacgtcttttgggggcaacctcggacgagcagtcttccaggccaaaaagagggttcttgaacctctgggcctggttgaggagcctgttaagacggctccgggaaaaaagaggccggtagagcactctcctgcggagccagactcctcctccggaaccggaaaagcgggccagcagcctgcaaaaaagagactgaatttcggtcagactggagactcagactccgtaccagaccctcaacctctcggagaaccaccagcagcccccacaagtttgggatctactacaatggcttcaggcggtggcgcaccagtggcagacaataacgagggtgccgatggagtgggtaattcctcgggaaattggcattgcgattcccaatggctgggcgacagagtcatcaccaccagcacccgaacctgggccctgcccacctacaacaaccatctgtacaaacagatttccagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttccttttactgcctggagtactttccttctcagatgctgcgtaccggaaacaacttccaattcagctacacctttgaggacgttcctttccacagcagctatgctcacagccagagtctggatcggctaatgaatcctctgatcgaccagtacctgtattatctgaacaagacacaatcagccagtggaactgttcagcagtctcggctactgtttagccaagctggacccacaagcatgtctcttcaagctaaaaactggctgcctggaccttgctacagacagcaacgtctgtcaaagcaggcaaacgaaaacaacaacagcaactttccctggactgccgctaccaagtatcatctaaatggccgggactcgttagttaatccaggaccgcctatggccagtcacaaggatgacgaagaaaagtttttccccatgcacggaaccctgatatttggtaaagaaggaacaaatgctaacaacgcggaattggaaaacgtcatgattacagatgaagaagaaatccgcaccacaaatcctgtagctacggagcagtacggcaatgtgtcaaataatttgcaaaactcaaatactgctccaagtactggaactgtcaatcaccaaggagcgttacctggtatggtgtggcaggatagagacgtgtacctgcagggacccatttgggccaagattcctcacaccgatggacattttcatccttctccgctgatgggaggttttggactcaaacatccgcctcctcagatcatgattaaaaacactcccgttccagccaatcctcctacaaacttcagtgcggcaaagtttgcttccttcatcacacagtattccacggggcaggtcagcgtggagatcgagtgggagctgcagaaggagaacagcaaacgctggaatcccgagatccagtacacttccaactacaacaaatctgttaatgtggactttactgtggacactaatggcgtgtattcagagcctcgccccattggcaccagatacctgactcgtaatctgtaa

SEQ ID NO: 54 - DNA, 2208 bp, adeno-associated virus 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 55 - protein, 733 aa, Adeno-associated virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 56 - protein, 736 aa, adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 57 - DNA, 22 bp, Artificial Sequence
  Rep_mpx1_F primer
cgcagacaggtaccaaaacaaa

SEQ ID NO: 58 - DNA, 20 bp, Artificial Sequence
  3UTR_mpx2_R primer
actccatcactaggggttcc

SEQ ID NO: 59 - DNA, 27 bp, Artificial Sequence
  AAV(excl5)rep_fwd primer
ggtaccaaaacaaatgttctcgtcacg

SEQ ID NO: 60 - DNA, 23 bp, Artificial Sequence
  AAV(prim)3ITR-D+G_R primer
ggagtggccaactccatcactag

SEQ ID NO: 61 - DNA, 23 bp, Artificial Sequence
  AAV(prim)3ITR-D+C_R primer
ggagtgggcaactccatcactag

SEQ ID NO: 62 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacacacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 63 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcaagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 64 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaagagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 65 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtgggctggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcgacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 66 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 67 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQQVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 68 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEEFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 69 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWAGATKYHLNGRDSLVNPGPAMASHKDDEEKFFPQSGVLIFGKQGSEKTNVDIEKVMITDEEEIRTTNPVATEQYGSVSTNLQRGNRQAATADVDTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 70 - protein, 736 aa, Artificial Sequence
  LK03 capsid polypeptide
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 71 - protein, 736 aa, Artificial Sequence
  CMRI_31 capsid polypeptide
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANSLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 72 - protein, 736 aa, Artificial Sequence
  CMRI_32 capsid polypeptide
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQQLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 73 - protein, 736 aa, Artificial Sequence
  CMRI_33 capsid polypeptide
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 74 - protein, 736 aa, Artificial Sequence
  CMRI_34 capsid polypeptide
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTETVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 75 - DNA, 40 bp, Artificial Sequence
  Reverse primer cDNA_R
gaaacgaattaaacggtttattgattaacaagcaattaca

SEQ ID NO: 76 - DNA, 24 bp, Artificial Sequence
  primer F_NGS
tctaggaactggcttcctggaccc

SEQ ID NO: 77 - DNA, 26 bp, Artificial Sequence
  Primer R_NGS
cagatgggcccctgaaggtacacatc

(19) United States
(12) Patent Application Publication
(10) Pub. No.: US 2022/0372512 A1
(43) Pub. Date: Nov. 24, 2022

(54) METHODS AND AAV VECTORS FOR IN VIVO TRANSDUCTION

(71) Applicants: Children's Medical Research Institute, Westmead (AU); The Sydney Children's Hospitals Network (Randwick and Westmead) (incorporating The Royal Alexandra, Westmead (AU)

(72) Inventors: Leszek LISOWSKI, Westmead (AU); Ian Edward ALEXANDER, Pennant Hills (AU); Marti CABANES CREUS, Westmead (AU); Claus Volker HALLWIRTH, Parramatta (AU); Samantha Lee GINN, Alexandria (AU)

(21) Appl. No.: 17/622,154

(22) PCT Filed: Jul. 3, 2020
(86) PCT No.: PCT/AU2020/050703

§ 371 Date: Dec. 22, 2021

(30) Foreign Application Priority Data
Jul. 4, 2019 (AU) 2019902374
Jul. 4, 2019 (AU) 2019902377
Oct. 22, 2019 (AU) 2019903974

(51) Int. Cl. C12N 15/86; C07K 14/005; A61P 1/16

(52) U.S. Cl. CPC C12N 15/86; C07K 14/005; A61P 1/16; C12N 2750/14171; C12N 2750/14122; C12N 2750/14152; C12N 2750/14143; A61K 48/00

(57) ABSTRACT

The present disclosure relates generally to methods and adeno-associated virus (AAV) vectors for in vivo transduction. The disclosure is directed to methods for producing modified AAV capsid polypeptides and AAV vectors that are suitable for transduction of cells in vivo, and methods for screening AAV vectors. The disclosure also relates to adeno-associated virus (AAV) capsid polypeptides and encoding nucleic acid molecules, AAV vectors comprising the capsid polypeptides, and nucleic acid vectors comprising the encoding nucleic acids molecules, as well as to host cells comprising the vectors. The disclosure also relates to methods and uses of the polypeptides, encoding nucleic acids molecules, vectors and host cells.

### RELATED APPLICATIONS

This application claims priority to Australian Provisional Application No. 2019902377 entitled “AAV capsid polypeptides and AAV vectors” filed 4 Jul. 2019, Australian Provisional Application No. 2019902374 entitled “Methods and AAV vectors for in vivo transduction” filed 4 Jul. 2019, and Australian Provisional Application No. 2019903974 entitled “Methods and AAV vectors for in vivo transduction” filed 22 Oct. 2019, the content of each of which is incorporated herein by reference in its entirety.

### FIELD OF THE DISCLOSURE

The present disclosure relates generally to methods and adeno-associated virus (AAV) vectors for in vivo transduction. The disclosure is in part directed to methods for producing modified AAV capsid polypeptides and AAV vectors that are suitable for transduction of cells in vivo, and methods for screening AAV vectors. The methods can provide for modified AAV capsid polypeptides that exhibit improved or enhanced in vivo transduction when present in an AAV vector. Thus, the disclosure is also directed to methods for producing AAV vectors with improved or enhanced in vivo transduction and methods for screening AAV vectors to identify those with improved or enhanced in vivo transduction. The disclosure also relates to adeno-associated virus (AAV) capsid polypeptides and encoding nucleic acid molecules, AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells.

### BACKGROUND OF THE DISCLOSURE

Gene therapy has most commonly been investigated and achieved using viral vectors, with notable recent advances being based on adeno-associated viral vectors. Adeno-associated virus (AAV) is a replication-deficient parvovirus, the single-stranded DNA genome of which is about 4.7 kb in length. The AAV genome includes inverted terminal repeat (ITRs) at both ends of the molecule, flanking two open reading frames: rep and cap. The cap gene encodes three capsid proteins: VP1, VP2 and VP3. The three capsid proteins typically assemble in a ratio of 1:1:8-10 to form the AAV capsid, although AAV capsids containing only VP3, or VP1 and VP3, or VP2 and VP3, have been produced. The cap gene also encodes the assembly activating protein (AAP) from an alternative open reading frame. AAP promotes capsid assembly, acting to target the capsid proteins to the nucleolus and promote capsid formation. The rep gene encodes four regulatory proteins: Rep78, Rep68, Rep52 and Rep40. These Rep proteins are involved in AAV genome replication.

The ITRs are involved in several functions, in particular integration of the AAV DNA into the host cell genome, as well as genome replication and packaging. When AAV infects a host cell, the viral genome can integrate into the host's chromosomal DNA resulting in latent infection of the cell. Thus, AAV can be exploited to introduce heterologous sequences into cells. In nature, a helper virus (for example, adenovirus or herpesvirus) provides protein factors that allow for replication of AAV virus in the infected cell and packaging of new virions. In the case of adenovirus, genes E1A, E1B, E2A, E4 and VA provide helper functions. Upon infection with a helper virus, the AAV provirus is rescued and amplified, and both AAV and the helper virus are produced.

AAV vectors (also referred to as recombinant AAV, rAAV) that contain a genome that lacks some, most or all of the native AAV genome and instead contains one or more heterologous sequences flanked by the ITRs have been successfully used in gene therapy settings. These AAV vectors are widely used to deliver heterologous nucleic acid to cells of a subject for therapeutic purposes, and in many instances, it is the expression of the heterologous nucleic acid that imparts the therapeutic effect. Although several AAV vectors have now been used in the clinic, there are a limited number that exhibit the required in vivo transduction efficiency in humans to facilitate adequate expression of the heterologous nucleic acid for therapeutic applications. There is therefore a need to develop methods for enhancing the in vivo transduction efficacy of AAV. There is also a need to identify and/or develop novel AAV vectors suitable for in vivo transduction, and to provide methods for identifying and/or developing such AAV vectors.

### SUMMARY OF THE DISCLOSURE

The present disclosure is predicated in part on the determination that the in vivo transduction efficiency of some AAV vectors, such as, but not limited to, AAV2, AAV3A, AAV3B, AAV13, AAV6, is related to their ability to bind heparin, whereby AAV vectors that bind heparin strongly typically exhibit inferior in vivo transduction efficiency, such as of human hepatocytes, compared to AAV vectors that bind heparin less strongly. Heparin is closely related to heparan sulphate proteoglycan (HSPG), which is present on the extracellular matrix of liver and other organs. Binding of AAV vectors to heparin is reflective of their ability to bind to HSPG. While not being bound by theory, it is postulated that, in vivo, HSPG molecules may be sequestering AAV vectors that bind too strongly to HSPG, reducing the ability of the vectors to access and transduce the desired target cells, such as hepatocytes in the liver. Thus, reducing binding of an AAV vector to heparin or HSPG can increase the in vivo transduction efficiency of the vector. This can be done, for example, by modifying the capsid polypeptide, e.g. substituting one or more amino acid residues that may contribute to the binding of the AAV capsid to HSPG.

Of significant interest to the field, the present inventors have also deduced for the first time that the prototypic AAV2 (isolate Srivastava/1982) having a capsid polypeptide set forth in SEQ ID NO:1 (Genbank Accession NC_001401.2; UniProtKB P03135) is likely a culture-adapted virus, exhibiting altered properties compared to “true” wild-type AAV2 isolates. While there is substantial evidence that liver is a major target organ for AAV2, including the identification of liver-specific enhancer-promoter activity in the 3′ untranslated region of prototypic culture-isolated AAV2 (Logan et al. Nat Genet. 2017 August; 49(8):1267-1273), when tested in vivo on human hepatocytes using the FRG mouse xenografted liver model, AAV vectors containing the prototypic capsid exhibit very poor transduction (see Examples below). The low in vivo transduction of primary human hepatocytes is supported by what was observed in a clinical trial for haemophilia B in which prototypic AAV2 vector was used and led to low expression of therapeutic protein (Manno et al. Nat Med. 2006 March; 12(3):342-7).

As demonstrated herein, AAV vectors comprising the prototypic AAV2 capsid have a high binding affinity for heparin. While this translates to effective transduction of human hepatocytes and derived cell lines in vitro, transduction of hepatocytes in vivo is relatively poor, possibly due to sequestration of vectors by HSPG in vivo before effective transduction of target cells is achieved, as discussed above. Notably, modification of the prototypic AAV2 capsid polypeptide at one or more amino acid positions identified herein as being involved in heparin/HSPG binding can reduce the ability of a vector comprising the now-modified capsid polypeptide to bind to heparin/HSPG and increase the in vivo transduction efficiency of the vector (compared to a vector containing the prototypic AAV2 capsid polypeptide). Moreover, as demonstrated herein, novel AAV2 capsid polypeptides identified from primary human liver samples can be vectorised to produce AAV vectors that have reduced binding to heparin/HSPG compared to a vector containing the prototypic AAV2 capsid polypeptide. Importantly, these AAV vectors are very effective at transducing human hepatocytes in vivo, while being less effective at in vitro transduction.

Thus, without being bound by theory, it is postulated that the prototypic AAV2 having a capsid comprising a polypeptide set forth in SEQ ID NO:1 is culture-adapted, having accumulated amino acid substitutions that increase binding affinity for heparin/HPSG. While this promotes infection/transduction of hepatocytes or hepatocyte-derived cell lines in vitro, it impairs infection/transduction of hepatocytes in vivo. Conversely, non-culture-adapted wild-type AAV2 isolates, such as those isolated directly from primary human livers, typically are very effective at infecting/transducing human hepatocytes in vivo.

The disclosure provided herein challenges the paradigm that AAV2 human hepatocyte entry is HSPG-mediated and demonstrates that natural human liver isolates, which lack some of the residues required for binding to HSPG, are remarkably efficient at targeting human hepatocytes in vivo. In addition to providing a mechanistic explanation for the weak performance of AAV2 in the first human liver directed clinical study, these findings provide powerful new insights for the ongoing development of AAV-based gene therapy: they potentially pave the way for the uptake of wild-type natural AAV2-like capsids into clinical applications targeting the human liver; and they suggest that enhanced HSPG binding can negatively affect the in vivo function of otherwise strongly hepatotropic variants and that modulation of the interaction with HSPG can ensure maximum efficiency in vivo.

Thus, in one aspect, the disclosure provides a method for enhancing the in vivo human hepatocyte transduction efficiency of an AAV vector, comprising: identifying a reference capsid polypeptide (e.g. an AAV2 or AAV2-like reference capsid) for transducing human hepatocytes in vivo; modifying the sequence of the reference capsid polypeptide to introduce one or more amino acid substitutions that alter the affinity of the capsid polypeptide for heparin, to thereby produce a modified capsid polypeptide; and vectorising the modified capsid polypeptide to thereby produce a modified AAV vector; wherein the modified AAV vector is characterized in that it elutes from a heparin affinity chromatography medium with a NaCl concentration of no more than 450 mM or with a conductivity of no more than 41 ms/cm; and has an in vivo transduction efficiency of hepatocytes (e.g. human hepatocytes) that is enhanced compared to a reference AAV vector having a capsid comprising the reference capsid polypeptide.

In another aspect, the disclosure provides a method for producing a modified AAV vector, such as a modified AAV vector for use in transducing human hepatocytes in vivo, comprising: identifying a reference capsid polypeptide (e.g. an AAV2 or AAV2-like reference capsid) for transducing human hepatocytes in vivo; modifying the sequence of the reference capsid polypeptide to introduce one or more amino acid substitutions that alter the affinity of the capsid polypeptide for heparin, to thereby produce a modified capsid polypeptide; and vectorising the modified capsid polypeptide to thereby produce a modified AAV vector; wherein the modified AAV vector is characterized in that it elutes from a heparin affinity chromatography medium with a NaCl concentration of no more than 450 mM or with a conductivity of no more than 41 ms/cm; and has an in vivo transduction efficiency of hepatocytes (e.g. human hepatocytes) that is enhanced compared to a reference AAV vector having a capsid comprising the reference capsid polypeptide.

In some embodiments, the modifying step comprises modifying the sequence of the reference capsid polypeptide to introduce one or more amino acid substitutions at an amino acid position corresponding to a position selected from among position 482, 484, 487, 496, 503, 532, 582, 585, 588, 589 and 596 of SEQ ID NO:1. In some examples, modification are made at two or more positions selected from among those corresponding positions 482, 496, 503, 532, 585, 588 and 596 of SEQ ID NO:1, e.g. at the amino acid positions corresponding to positions 482 and 496; 482 and 532; 482, 496 and 532; 482 and 585; 482, 503 and 596; 496, 588 and 596; 496, 532, 585, 588 and 596; or 503, 585, 588 and 596 of SEQ ID NO:1. In some examples, the one or more amino acid substitutions is selected from among: substitution of the amino acid residue at the position corresponding to position 482 of SEQ ID NO:1 with a serine (S) or threonine (T); substitution of the amino acid residue at the position corresponding to position 484 of SEQ ID NO:1 with a glutamine (Q) or asparagine (N); substitution of the amino acid residue at the position corresponding to position 487 of SEQ ID NO:1 with a glutamine (Q) or asparagine (N); substitution of the amino acid residue at the position corresponding to position 496 of SEQ ID NO:1 with an aspartic acid (D) or glutamic acid (E); substitution of the amino acid residue at the position corresponding to position 503 of SEQ ID NO:1 with an alanine (A), valine (V) or isoleucine (I); substitution of the amino acid residue at the position corresponding to position 532 of SEQ ID NO:1 with a glutamic acid (E) or glutamine (Q); substitution of the amino acid residue at the position corresponding to position 582 of SEQ ID NO:1 with a serine (S), aspartic acid (D), tyrosine (Y), or threonine (T); substitution of the amino acid residue at the position corresponding to position 585 of SEQ ID NO:1 with a serine (S), threonine (T), glycine (G), alanine (A) or glutamic acid (E); substitution of the amino acid residue at the position corresponding to position 588 of SEQ ID NO:1 with a threonine (T), isoleucine (I) or alanine (A); substitution of the amino acid residue at the position corresponding to position 589 of SEQ ID NO:1 with an aspartic acid (D), glutamic acid (E) or alanine (A); and substitution of the amino acid residue at the position corresponding to position 596 of SEQ ID NO:1 with an aspartic acid (D) or glutamic acid (E) (e.g. C482S, C482T, R484Q, R484N, R487Q, R487N, N496D, N496E, T503A, T503V, T503I, K532E, K532Q, N582S, N582D, N582Y, N582T, R585S, R585T, R585G, R585A, R585E, R588T, R588I, R588A, Q589D, Q589E, Q589A, N596D or N596E relative to SEQ ID NO:1). In some examples, step b) comprises modifying the sequence of the reference capsid polypeptide to introduce two or more amino acid substitutions, and wherein the two or more amino acid substitutions comprise: a serine (S) at the position corresponding to position 482 and an aspartic acid (D) at the position corresponding to position 496 (e.g. C482S and N496D relative to SEQ ID NO:1); a serine (S) at the position corresponding to position 482 and a glutamine (Q) at the position corresponding to position 532 (e.g. C482S and K532Q relative to SEQ ID NO:1); a serine (S) at the position corresponding to position 482, an aspartic acid (D) at the position corresponding to position 496 and a glutamine (Q) at the position corresponding to position 532 (e.g. C482S, N496D and K532Q relative to SEQ ID NO:1); a serine (S) at the position corresponding to position 482 and a serine (S) at the position corresponding to position 585 (e.g. C482S and R585S relative to SEQ ID NO:1); a serine (S) at the position corresponding to position 482, an alanine (A) at the position corresponding to position 503 and an aspartic acid (D) at the position corresponding to position 596 (e.g. C482S, T503A and N596D relative to SEQ ID NO:1); an alanine (A) at the position corresponding to position 503 and an aspartic acid (D) at the position corresponding to position 596 (e.g. T503A and N596D relative to SEQ ID NO:1); an aspartic acid (D) at the position corresponding to position 496, a glutamine (Q) at the position corresponding to position 532, a serine (S) at the position corresponding to position 585, a threonine (T) at the position corresponding to position 588 and an aspartic acid (D) at the position corresponding to position 596 (e.g. N496D, K532Q, R585S, R588T and N596D relative to SEQ ID NO:1); an aspartic acid (D) at the position corresponding to position 496, a threonine (T) at the position corresponding to position 588 and an aspartic acid (D) at the position corresponding to position 596 (e.g. N496D, R588T and N596D relative to SEQ ID NO:1); or an alanine (A) at the position corresponding to position 503, a serine (S) at the position corresponding to position 585, a threonine (T) at the position corresponding to position 588 and an aspartic acid (D) at the position corresponding to position 596 (e.g. T503A, R585S, R588T and N596D relative to SEQ ID NO:1). In particular examples, the reference capsid polypeptide comprises the sequence set forth in SEQ ID NO:1 or a sequence having at least or about 90% sequence identity thereto (i.e. is an AAV2 or AAV2-like capsid polypeptide).

The disclosure also provides a method for producing a modified AAV vector (e.g. for use in transducing human hepatocytes in vivo) or for enhancing in vivo transduction efficiency of an AAV vector, comprising: identifying a reference capsid polypeptide (e.g. an AAV3B or AAV3B-like reference capsid) for transducing human hepatocytes in vivo; modifying the sequence of a reference capsid polypeptide to introduce one or more amino acid substitutions that alter the affinity of the capsid polypeptide for heparin, to thereby produce a modified capsid polypeptide; and vectorising the modified capsid polypeptide to thereby produce a modified AAV vector; wherein the modified AAV vector is characterized in that it elutes from a heparin affinity chromatography medium with a NaCl concentration of no more than 275 mM or with a conductivity of no more than 25 ms/cm; and has an in vivo transduction efficiency of hepatocytes (e.g. human hepatocytes) that is enhanced compared to a reference AAV vector having a capsid comprising the reference capsid polypeptide.

In particular embodiments, the modifying step comprises modifying the sequence of the reference capsid polypeptide to introduce one or more amino acid substitutions at an amino acid position corresponding to a position selected from among position 476, 485, 488, 533, 594 and 598 of SEQ ID NO:56. In some examples, at least two amino acid substitutions at amino acid positions corresponding to positions 594 and 598 of SEQ ID NO:56 are made. In some examples, the one or more amino acid substitutions is selected from among: substitution of the amino acid residue at the position corresponding to position 476 of SEQ ID NO:56 with a glutamine (Q) or asparagine (N); substitution of the amino acid residue at the position corresponding to position 485 of SEQ ID NO:56 with a glutamine (Q) or asparagine (N); substitution of the amino acid residue at the position corresponding to position 488 of SEQ ID NO:56 with a glutamine (Q) or asparagine (N); substitution of the amino acid residue at the position corresponding to position 533 of SEQ ID NO:56 with a glutamic acid (E) or glutamine (Q); substitution of the amino acid residue at the position corresponding to position 598 of SEQ ID NO:56 with a glutamic acid (E) or histidine (H); and substitution of the amino acid residue at the position corresponding to position 594 of SEQ ID NO:56 with a glycine (G), alanine (A) or glutamic acid (E) (e.g. R476Q, R476N, R485Q, R485N, R488Q, R488N, K533E, K533Q, D598E, D598H, R594G, R594E, or R594A relative to SEQ ID NO:56). In one examples, the sequence of the reference capsid polypeptide to introduce amino acid substitutions R594E and D598H. In illustrative embodiments, the reference capsid polypeptide comprises the sequence set forth in SEQ ID NO:56 or a sequence having at least or about 90% sequence identity thereto (i.e. is an AAV3B or AAV3B-like capsid polypeptide).

In further embodiments, the disclosure provides a method for producing a modified AAV vector (e.g. for use in transducing human hepatocytes in vivo) or for enhancing in vivo transduction efficiency of an AAV vector, comprising: identifying a reference capsid polypeptide (e.g. an AAV13 or AAV13-like reference capsid) for transducing human hepatocytes in vivo; modifying the sequence of a reference capsid polypeptide to introduce one or more amino acid substitutions that alter the affinity of the capsid polypeptide for heparin, to thereby produce a modified capsid polypeptide; and vectorising the modified capsid polypeptide to thereby produce a modified AAV vector; wherein the modified AAV vector is characterized in that it elutes from a heparin affinity chromatography medium with a NaCl concentration of no more than 330 mM or with a conductivity of no more than 30 ms/cm; and has an in vivo transduction efficiency of hepatocytes (e.g. human hepatocytes) that is enhanced compared to a reference AAV vector having a capsid comprising the reference capsid polypeptide.

In some embodiments, the modifying step comprises modifying the sequence of the reference capsid polypeptide to introduce one or more amino acid substitutions at an amino acid position corresponding to a position selected from among 482, 485, 525, 528 and 530 of SEQ ID NO:55. For example, the one or more amino acid substitutions may be selected from among: substitution of the amino acid residue at the position corresponding to position 482 of SEQ ID NO:55 with a glutamine (Q) or asparagine (N); substitution of the amino acid residue at the position corresponding to position 485 of SEQ ID NO:55 with a glutamine (Q) or asparagine (N); substitution of the amino acid residue at the position corresponding to position 525 of SEQ ID NO:55 with a glutamic acid (E) or glutamine (Q); substitution of the amino acid residue at the position corresponding to position 528 of SEQ ID NO:3 with a glutamic acid (E) or glutamine (Q); and substitution of the amino acid residue at the position corresponding to position 530 of SEQ ID NO:55 with a glutamic acid (E) or glutamine (Q) (e.g. R482Q, R482N, R485Q, R485N, K525E, K525Q, K528E, K528Q K530E or K530Q relative to SEQ ID NO:55). In particular embodiments, the reference capsid polypeptide comprises the sequence set forth in SEQ ID NO:55 or a sequence having at least or about 90% sequence identity thereto (i.e. is an AAV13 or AAV13-like capsid polypeptide).

In some examples of the above methods, at least 1, 2, 3, 4, 5, 6, 7, 8, 9, 10 or 11 amino acid substitutions are introduced. In further examples, the modified AAV vector has an in vivo transduction efficiency that is enhanced by at least or about 10%, 20%, 30%, 40%, 50%, 60%, 70%, 80%, 90%, 100%, 150%, 200%, 300% or more compared to the reference AAV vector. The methods may also optionally comprise performing a binding assay to assess the binding of the modified AAV vector to an affinity chromatography medium to which heparin, heparan sulphate and/or heparan sulphate proteoglycan is attached. The methods may also optionally comprise assessing the transduction efficiency of the modified AAV vector in vivo system that utilises human hepatocytes (e.g. a small animal (e.g. a mouse) with a chimeric liver comprising human hepatocytes, such as a hFRG mouse).

In another aspect, the disclosure provides a method for identifying a candidate AAV vector that is suitable for in vivo transduction of human cells (e.g. human hepatocytes), comprising assessing the heparin binding capacity of a test AAV vector and comparing the heparin binding capacity of the test AAV vector to the heparin binding capacity of a reference AAV vector, wherein: the test AAV vector is an AAV2 or AAV2-like vector comprising a capsid polypeptide comprising the sequence set forth in SEQ ID NO:1 or a sequence having at least or about 90% sequence identity thereto; the reference AAV vector elutes from a heparin affinity chromatography medium with a NaCl concentration at least 450 mM or with a conductivity of at least 41 ms/cm; and when the test AAV vector has a heparin binding capacity below the reference AAV vector, the test AAV vector is identified as a candidate AAV vector suitable for in vivo transduction of human cells (e.g. human hepatocytes).

In a further aspect, the disclosure provides a method for identifying a candidate AAV vector that is suitable for in vivo transduction of human cells (e.g. human hepatocytes), comprising assessing the heparin binding capacity of a test AAV vector and comparing the heparin binding capacity of the test AAV vector to the heparin binding capacity of a reference AAV vector, wherein: the test AAV vector is an AAV3 or AAV3-like vector comprising a capsid polypeptide comprising the sequence set forth in SEQ ID NO:56 or a sequence having at least or about 90% sequence identity thereto; the reference AAV vector elutes from a heparin affinity chromatography medium with a NaCl concentration at least 275 mM or with a conductivity of at least 25 ms/cm; and when the test AAV vector has a heparin binding capacity below the reference AAV vector, the test AAV vector is identified as a candidate AAV vector suitable for in vivo transduction of human cells (e.g. human hepatocytes).

In a still further aspect, the disclosure provides a method for identifying a candidate AAV vector that is suitable for in vivo transduction of human cells (e.g. human hepatocytes), comprising assessing the heparin binding capacity of a test AAV vector and comparing the heparin binding capacity of the test AAV vector to the heparin binding capacity of a reference AAV vector, wherein: the test AAV vector is an AAV13 or AAV13-like vector comprising a capsid polypeptide comprising the sequence set forth in SEQ ID NO:55 or a sequence having at least or about 90% sequence identity thereto; the reference AAV vector elutes from a heparin affinity chromatography medium with a NaCl concentration at least 330 mM or with a conductivity of at least 30 ms/cm; and when the test AAV vector has a heparin binding capacity below the reference AAV vector, the test AAV vector is identified as a candidate AAV vector suitable for in vivo transduction of human cells (e.g. human hepatocytes).

In some embodiment of the methods for identifying a candidate AAV vector, the methods further comprise assessing the ability of the candidate AAV vector to transduce human cells in vivo (e.g. using small animal (e.g. a mouse) with a chimeric liver comprising human hepatocytes, such as a hFRG mouse). In other embodiments, assessing the heparin binding capacity comprises performing a binding assay to assess the binding of the modified AAV vector to an affinity chromatography medium to which heparin, heparan sulphate and/or heparan sulphate proteoglycan is attached. In some examples of these methods, the cells are hepatocytes. In further examples, the candidate AAV vector is produced according to the methods described above and herein for producing a modified AAV vector.

A further aspect of the disclosure relates to an AAV vector that is produced by the methods described above and herein for producing a modified AAV vector.

In another aspect, the disclosure provides a method for delivering a heterologous coding sequence to a hepatocyte in a human subject, comprising administering to human subject an AAV vector comprising a heterologous coding sequence, wherein the AAV vector comprises a capsid comprising a wild-type AAV2 capsid polypeptide comprising an amino acid sequence having at least 92% sequence identity to the polypeptide set forth in SEQ ID NO:5.

In some embodiments, the capsid polypeptide comprises an amino acid residue at the position corresponding to position 585 of SEQ ID NO:5 that is not an arginine. In other embodiments, the capsid polypeptide comprises an amino acid residue at the position corresponding to position 588 of SEQ ID NO:5 that is not an arginine.

In particular embodiments of the method for delivering a heterologous coding sequence, the human subject has a liver-associated disease treatable by the in vivo expression of the heterologous coding sequence. The liver-associated disease may be, for example, a urea cycle disorder (UCD), organic acidopathy, amino acidopathy, glycogenoses (Types I, III and IV), Wilson's disease, Progressive Familial Intrahepatic Cholestasis, primary hyperoxaluria, complementopathy, coagulopathy, Crigler Najjar syndrome, familial hypercholesterolaemia, α-1-antitrypsin deficiency, mitochondria respiratory chain hepatopathy, or citrin deficiency. In such examples, the heterologous coding sequence may comprise all or a part of a gene selected from among OTC, ASS, CPS1, ASL, ARG1, PCCA, PCCB, MMUT, PAH, FAH, SLC37A4, ATP7B, ABCB4, ABCB11, ATP8B1, AGXT, CFH, CFI, F8, F9, UGT1A1, LDLR, SERPINA1, POLG and SLC25A13; and/or encodes for a protein encoded by a gene selected from among OTC, ASS, CPS1, ASL, ARG1, PCCA, PCCB, MMUT, PAH, FAH, SLC37A4, ATP7B, ABCB4, ABCB11, ATP8B1, AGXT, CFH, CFI, F8, F9, UGT1A1, LDLR, SERPINA1, POLG and SLC25A13.

In a further aspect, the disclosure is directed to the use of an AAV vector in the preparation of a medicament for treating a liver-associated disease in a human subject, wherein: the AAV vector comprises a heterologous coding sequence; the AAV vector comprises a capsid comprising a wild-type AAV2 capsid polypeptide comprising an amino acid sequence having at least 92% sequence identity to the polypeptide set forth in SEQ ID NO:5; and the liver-associated disease is treatable by the in vivo expression of the heterologous coding sequence. In some embodiments, the capsid polypeptide comprises an amino acid residue at the position corresponding to position 585 of SEQ ID NO:5 that is not an arginine. In further embodiments, the capsid polypeptide comprises an amino acid residue at the position corresponding to position 588 of SEQ ID NO:5 that is not an arginine.

In particular embodiments of this use, the human subject has a liver-associated disease treatable by the in vivo expression of the heterologous coding sequence. The liver-associated disease may be, for example, a urea cycle disorder (UCD), organic acidopathy, amino acidopathy, glycogenoses (Types I, III and IV), Wilson's disease, Progressive Familial Intrahepatic Cholestasis, primary hyperoxaluria, complementopathy, coagulopathy, Crigler Najjar syndrome, familial hypercholesterolaemia, α-1-antitrypsin deficiency, mitochondria respiratory chain hepatopathy, or citrin deficiency. In such examples, the heterologous coding sequence may comprise all or a part of a gene selected from among OTC, ASS, CPS1, ASL, ARG1, PCCA, PCCB, MMUT, PAH, FAH, SLC37A4, ATP7B, ABCB4, ABCB11, ATP8B1, AGXT, CFH, CFI, F8, F9, UGT1A1, LDLR, SERPINA1, POLG and SLC25A13; and/or encodes for a protein encoded by a gene selected from among OTC, ASS, CPS1, ASL, ARG1, PCCA, PCCB, MMUT, PAH, FAH, SLC37A4, ATP7B, ABCB4, ABCB11, ATP8B1, AGXT, CFH, CFI, F8, F9, UGT1A1, LDLR, SERPINA1, POLG and SLC25A13.

The present disclosure is also predicated in part on the identification of novel AAV capsid polypeptides. In particular embodiments, the capsid polypeptides facilitate efficient transduction of human cells (such as human hepatocytes) when contained in an AAV vector. Typically, the in vivo transduction efficiency of AAV vectors comprising a capsid polypeptide of the present disclosure is significantly improved compared to AAV vectors comprising other AAV capsid polypeptides (e.g. one set forth in SEQ ID NO:1, 55 or 56). The capsids polypeptides of the present disclosure are therefore particularly useful in preparing AAV vectors, and in particular, AAV vectors for gene therapy uses. Similarly, AAV vectors comprising a capsid polypeptide of the present disclosure (i.e. having a capsid comprising or consisting of a capsid polypeptide of the present disclosure) are of particular use in gene therapy applications, such as for delivery of heterologous nucleic acids for the treatment of various diseases and conditions.

In one aspect, the disclosure provides an isolated capsid polypeptide, comprising: the sequence of amino acids set forth in any one of SEQ ID Nos:2-27, 62-65 and 71-74 or a sequence having at least or about 90% sequence identity thereto; the sequence of amino acids at positions 138-735 of any one of SEQ ID NOs:2, 3, 5-11, 19-27 and 62-65, positions 138-734 of SEQ ID NO:4 or 24, positions 138-736 of SEQ ID NO:16 and 71-74, or positions 138-737 of any one of SEQ ID NOs:12-15, 17 and 18; or a sequence having at least or about 90% sequence identity thereto; and/or the sequence of amino acids at positions 203-735 of any one of SEQ ID Nos:2, 3, 5-11, 19-27 and 62-65, positions 203-734 of SEQ ID NO:4, positions 204-736 of SEQ ID NO:16 and 71-74, or positions 204-737 of any one of SEQ ID NOs:12-15, 17 and 18; or a sequence having at least or about 90% sequence identity thereto.

In another aspect, the disclosure provides an isolated capsid polypeptide, comprising a sequence of amino acids comprising one or more amino acid substitutions relative to the AAV2 capsid polypeptide set forth in SEQ ID NO:1 or a polypeptide having at least or about 90% sequence identity to the polypeptide set forth in SEQ ID NO:1, wherein the one or more amino acid substitutions are at an amino acid position selected from among those corresponding to positions 482, 484, 487, 496, 503, 532, 582, 585, 588, 589 and 596 of the AAV2 capsid polypeptide set forth in SEQ ID NO:1. In one example, the polypeptides comprises two or more amino acid substitutions at amino acid positions selected from among those corresponding positions 482, 496, 503, 532, 585, 588 and 596 of SEQ ID NO:1, e.g. at the amino acid positions corresponding to positions 482 and 496; 482 and 532; 482, 496 and 532; 482 and 585; 482, 503 and 596; 496, 588 and 596; 496, 532, 585, 588 and 596; or 503, 585, 588 and 596 of SEQ ID NO:1.

In some embodiments, the isolated capsid polypeptides comprises 1, 2, 3, 4, 5, 6, 7, 8, 9, 10 or 11 amino acid substitutions at one or more positions selected from among those corresponding to positions 482, 484, 487, 496, 503, 532, 582, 585, 588, 589 and 596 of the AAV2 capsid polypeptide set forth in SEQ ID NO:1. In a particular embodiment, the one or more amino acid substitutions are selected from among: substitution of the amino acid residue at the position corresponding to position 482 of SEQ ID NO:1 with a serine (S) or threonine (T); substitution of the amino acid residue at the position corresponding to position 484 of SEQ ID NO:1 with a glutamine (Q) or asparagine (N); substitution of the amino acid residue at the position corresponding to position 487 of SEQ ID NO:1 with a glutamine (Q) or asparagine (N); substitution of the amino acid residue at the position corresponding to position 496 of SEQ ID NO:1 with an aspartic acid (D) or glutamic acid (E); substitution of the amino acid residue at the position corresponding to position 503 of SEQ ID NO:1 with an alanine (A), valine (V) or isoleucine (I); substitution of the amino acid residue at the position corresponding to position 532 of SEQ ID NO:1 with a glutamic acid (E) or glutamine (Q); substitution of the amino acid residue at the position corresponding to position 582 of SEQ ID NO:1 with a serine (S), aspartic acid (D), tyrosine (Y), or threonine (T); substitution of the amino acid residue at the position corresponding to position 585 of SEQ ID NO:1 with a serine (S), threonine (T), glycine (G), alanine (A) or glutamic acid (E); substitution of the amino acid residue at the position corresponding to position 588 of SEQ ID NO:1 with a threonine (T), isoleucine (I) or alanine (A); substitution of the amino acid residue at the position corresponding to position 589 of SEQ ID NO:1 with an aspartic acid (D), glutamic acid (E) or alanine (A); and substitution of the amino acid residue at the position corresponding to position 596 of SEQ ID NO:1 with an aspartic acid (D) or glutamic acid (E). For example, the one or more amino acid substitutions may be selected from among C482S, C482T, R484Q, R484N, R487Q, R487N, N496D, N496E, T503A, T503V, T503I, K532E, K532Q, N582S, N582D, N582Y, N582T, R585S, R585T, R585G, R585A, R585E, R588T, R588I, R588A, Q589D, Q589E, Q589A, N596D and N596E, relative to SEQ ID NO:1. In an illustrative embodiment, the capsid polypeptide comprises: a serine (S) at the position corresponding to position 482 and an aspartic acid (D) at the position corresponding to position 496 (e.g. C482S and N496D relative to SEQ ID NO:1); a serine (S) at the position corresponding to position 482 and a glutamine (Q) at the position corresponding to position 532 (e.g. C482S and K532Q); a serine (S) at the position corresponding to position 482, an aspartic acid (D) at the position corresponding to position 496 and a glutamine (Q) at the position corresponding to position 532 (e.g. C482S, N496D and K532Q relative to SEQ ID NO:1); a serine (S) at the position corresponding to position 482 and a serine (S) at the position corresponding to position 585 (e.g. C482S and R585S relative to SEQ ID NO:1); a serine (S) at the position corresponding to position 482, an alanine (A) at the position corresponding to position 503 and an aspartic acid (D) at the position corresponding to position 596 (e.g. C482S, T503A and N596D relative to SEQ ID NO:1); an alanine (A) at the position corresponding to position 503 and an aspartic acid (D) at the position corresponding to position 596 (e.g. T503A and N596D relative to SEQ ID NO:1); an aspartic acid (D) at the position corresponding to position 496, a glutamine (Q) at the position corresponding to position 532, a serine (S) at the position corresponding to position 585, a threonine (T) at the position corresponding to position 588 and an aspartic acid (D) at the position corresponding to position 596 (e.g. N496D, K532Q, R585S, R588T and N596D relative to SEQ ID NO:1); an aspartic acid (D) at the position corresponding to position 496, a threonine (T) at the position corresponding to position 588 and an aspartic acid (D) at the position corresponding to position 596 (e.g. N496D, R588T and N596D relative to SEQ ID NO:1); or an alanine (A) at the position corresponding to position 503, a serine (S) at the position corresponding to position 585, a threonine (T) at the position corresponding to position 588 and an aspartic acid (D) at the position corresponding to position 596 (e.g. T503A, R585S, R588T and N596D relative to SEQ ID NO:1).

In a further aspect, provided is an isolated capsid polypeptide, comprising a sequence of amino acids comprising one or more amino acid substitutions relative to the AAV3B capsid polypeptide set forth in SEQ ID NO:56 or a polypeptide having at least or about 90% sequence identity to the polypeptide set forth in SEQ ID NO:56, wherein the one or more amino acid substitutions are at an amino acid position selected from among those corresponding to positions 476, 485, 488, 533, 594 and 598 of the AAV3B capsid polypeptide set forth in SEQ ID NO:56. In one example, the polypeptide comprises amino acid substitutions at positions corresponding to positions 594 and 598 of the AAV3B capsid polypeptide set forth in SEQ ID NO:56.

In one embodiment, the isolated capsid polypeptide comprises 1, 2, 3, 4, 5, or 6 amino acid substitutions at one or more positions selected from among those corresponding to positions 476, 485, 488, 533, 594 and 598 of the AAV3B capsid polypeptide set forth in SEQ ID NO:56. In a particular embodiment, the one or more amino acid substitutions is selected from among substitution of the amino acid residue at the position corresponding to position 476 of SEQ ID NO:56 with a glutamine (Q) or asparagine (N); substitution of the amino acid residue at the position corresponding to position 485 of SEQ ID NO:56 with a glutamine (Q) or asparagine (N); substitution of the amino acid residue at the position corresponding to position 488 of SEQ ID NO:56 with a glutamine (Q) or asparagine (N); substitution of the amino acid residue at the position corresponding to position 533 of SEQ ID NO:56 with a glutamic acid (E) or glutamine (Q); substitution of the amino acid residue at the position corresponding to position 598 of SEQ ID NO:56 with a glutamic acid (E) or histidine (H); and substitution of the amino acid residue at the position corresponding to position 594 of SEQ ID NO:56 with a glycine (G), glutamic acid (E) or alanine (A). For example, the one or more amino acid substitutions may be selected from among R476Q, R476N, R485Q, R485N, R488Q, R488N, K533E, K533Q, D598E, D598H, R594G, R594E and R594A relative to SEQ ID NO:56. In one examples, the polypeptide comprises a substitution of the amino acid residue at the position corresponding to position 594 of SEQ ID NO:56 with a glutamic acid (E) and substitution of the amino acid residue at the position corresponding to position 598 of SEQ ID NO:56 with a histidine (H) (e.g. R594E and D598H relative to SEQ ID NO:56).

In a further aspect, provided is an isolated capsid polypeptide, comprising a sequence of amino acids comprising one or more amino acid substitutions relative to the AAV13 capsid polypeptide set forth in SEQ ID NO:55 or a polypeptide having at least or about 90% sequence identity to the polypeptide set forth in SEQ ID NO:55, wherein the one or more amino acid substitutions are at an amino acid position selected from among those corresponding to positions 482, 485, 525, 528 and 530 of the AAV13 capsid polypeptide set forth in SEQ ID NO:55.

In one embodiment, the isolated capsid polypeptide comprises 1, 2, 3, 4 or 5 amino acid substitutions at one or more positions selected from among those corresponding to positions 482, 485, 525, 528 and 530 of the AAV13 capsid polypeptide set forth in SEQ ID NO:55. In a particular embodiment, the one or more amino acid substitutions is selected from among: substitution of the amino acid residue at the position corresponding to position 482 of SEQ ID NO:55 with a glutamine (Q) or asparagine (N); substitution of the amino acid residue at the position corresponding to position 485 of SEQ ID NO:55 with a glutamine (Q) or asparagine (N); substitution of the amino acid residue at the position corresponding to position 525 of SEQ ID NO:55 with a glutamic acid (E) or glutamine (Q); substitution of the amino acid residue at the position corresponding to position 528 of SEQ ID NO:55 with a glutamic acid (E) or glutamine (Q); and substitution of the amino acid residue at the position corresponding to position 530 of SEQ ID NO:55 with a glutamic acid (E) or glutamine (Q). For example, the one or more amino acid substitutions may be selected from among R482Q, R482N, R485Q, R485N, K525E, K525Q, K528E, K528Q K530E and K530Q relative to SEQ ID NO:55. In some examples, the capsid polypeptide comprises a substitution of the amino acid residue at the position corresponding to position 594 of SEQ ID NO:56 with a glutamic acid (E) and a substitution of the amino acid residue at the position corresponding to position 598 of SEQ ID NO:56 with a histidine (H), e.g. the amino acid substitutions R594E and D598H relative to SEQ ID NO:56.

Also provided is an AAV vector, comprising a capsid polypeptide described above or herein. In some examples, the AAV vector comprises a heterologous coding sequence, e.g. one that encodes a peptide, polypeptide or polynucleotide.

In further aspects, provided is an isolated nucleic acid molecule encoding a capsid polypeptide described above or herein, and a vector comprising the nucleic acid molecule. In some examples, the vector is selected from among a plasmid, cosmid, phage and transposon. A host cell comprising an AAV vector, a nucleic acid molecule or a vector described above and herein is also provided.

Also provided is a method for introducing a heterologous coding sequence into a host cell, comprising contacting a host cell with the AAV vector of the present disclosure that comprises a heterologous coding sequence. In some examples, the host cell is a hepatocyte.

In another aspect, provided is a method for producing an AAV vector, comprising culturing a host cell comprising a nucleic acid molecule encoding a capsid polypeptide of the present disclosure, an AAV rep gene, a heterologous coding sequence flanked by AAV inverted terminal repeats, and helper functions for generating a productive AAV infection, under conditions suitable to facilitate assembly of an AAV vector comprising a capsid comprising the capsid polypeptide, wherein the capsid encapsidates the heterologous coding sequence. In some examples, the host cell is a hepatocyte.

### BRIEF DESCRIPTION OF THE DRAWINGS

Embodiments of the disclosure are described herein, by way of non-limiting example only, with reference to the following drawings.

FIG. 1 is a graphical representation of the in vivo transduction of hepatocytes by various AAV vectors. An hFRG mouse was injected with 5.0×10¹⁰ barcoded vector genomes of each of the AAV vectors. One week after injection the chimeric liver of the mouse was perfused and human and murine hepatocytes were single cell sorted. DNA and RNA were recovered from the human population of hepatocytes and Next Generation Sequencing (NGS) of the barcode region in the transgene in each of the AAV vectors was performed. DNA and RNA specific for the barcodes and thus each vector was then quantified and shown here as a percentage of the total AAV-derived DNA and cDNA populations.

FIG. 2 is a graphical representation of the in vivo transduction of hepatocytes by various AAV vectors. (A) In vivo transduction of hepatocytes by the CMRI_18 AAV variant. An hFRG mouse was injected intravenously with 2.00×10¹¹ vg barcoded vector genomes of each of the CMRI_18, AAV2 and AAV8 vectors. Percentage of the total AAV-derived DNA is shown. (B)-(D) In vivo physical and functional transduction comparison of in vivo adapted AAV2 variants using xenograft liver model. Each of the AAV variants were used to package six barcoded ssAAV-LSP1-GFP-BC (1-6)-WPRE-BGHpA constructs. AAV2, AAV8, AAV-LK03 and AAV13 were used as controls. Equimolar mix of all seven variants was injected intravenously into two humanized FRG mice (total 3.5×10¹¹ vg/mouse) (n=6 BCs×7 variants=42 BCs total). Percentage of NGS reads mapped to each capsid in human cells at the DNA (375.56 vgs/diploid human genome) and RNA level, normalised to the injection mix, is shown. Immunohistochemical analysis of liver was also performed: (B) DNA specific for the vectors in human hepatocytes, representing hepatocyte entry. (C) cDNA specific for the vectors in human hepatocytes, representing hepatocyte expression. (D) Immunohistochemical analysis. Quantification of the percentage of transduced human hepatocytes per human cluster. Data shown as mean+SD, n=10 human clusters/mouse, n=1 mouse/vector. Statistical significances were calculated using the two Mann-Whitney test. P value <0.0001 (****); n.s.=non-significant (p value >0.05).

FIG. 3 is a graphical representation of the in vivo transduction of hepatocytes by various AAV vectors. An hFRG mouse was injected with each of the AAV vectors packaged with a barcoded transgene harbouring a Liver Specific Promoter driving eGFP (2 barcodes/capsid). One week after injection the chimeric liver of the mouse was perfused and human and murine hepatocytes were single cell sorted. DNA and RNA were recovered from the human population of hepatocytes and Next Generation Sequencing (NGS) of the barcoded transgene in each of the AAV vectors was performed. DNA and RNA specific for the barcodes, and thus each AAV variant, was then quantified before being normalised to preinjection reads (A) DNA specific for the vectors in human hepatocytes, representing hepatocyte entry. (B) cDNA specific for the vectors in human hepatocytes, representing hepatocyte expression.

FIG. 4 is a graphical representation of the in vivo transduction of hepatocytes by various AAV vectors. An hFRG mouse was injected with each of the AAV vectors packaged with a barcoded transgene harbouring a Liver Specific Promoter driving eGFP. One week after injection the chimeric liver of the mouse was perfused and human and murine hepatocytes were single cell sorted. DNA and RNA were recovered from the human population of hepatocytes and Next Generation Sequencing (NGS) of the barcoded transgene in each of the AAV vectors was performed. DNA and RNA specific for the barcodes, and thus each AAV variant, was then quantified and is shown here as a percentage of the total NGS sequencing reads (DNA and cDNA). The initial vector mix was also deep sequenced for validation and is shown on the preinjection graph.

FIG. 5 is a graphical representation of the in vivo transduction of hepatocytes by various AAV vectors. An hFRG mouse was injected with each of the AAV vectors (5 barcoded transgenes/capsid). One week after injection the chimeric liver of the mouse was perfused and human and murine hepatocytes were single cell sorted. DNA was recovered from both the human and the murine populations of hepatocytes and Next Generation Sequencing (NGS) of the barcoded transgene in each of the AAV vectors was performed. DNA and RNA specific for the barcodes, and thus each vector, was then quantified and is shown here as a percentage of the total NGS sequencing reads. The initial vector mix was also deep sequenced for validation. (A) DNA specific for the vectors in the preinjection mix. (B) DNA specific for the vectors in human hepatocytes. (C) DNA specific for the vectors in mouse hepatocytes. (D) cDNA (RNA) specific for the vectors in human hepatocytes. (E) cDNA (RNA) specific for the vectors in mouse hepatocytes.

FIG. 6 is a graphical representation of the in vivo transduction of hepatocytes by various AAV vectors. An hFRG mouse was injected with each of the AAV vectors. One week after injection the chimeric liver of the mouse was perfused and human and murine hepatocytes were single cell sorted. DNA was recovered from the human population of hepatocytes and Next Generation Sequencing (NGS) of the barcoded transgene in each of the AAV vectors was performed. DNA specific for the barcodes and thus each vector was then quantified and is shown here as a percentage of the total NGS sequencing reads. (A) Entry index ((499.3 vgs/diploid 653 cell). (B) Expression index. (C) Immunohistochemical analysis. Quantification of the percentage of transduced human hepatocytes per human cluster. Data shown as mean+SD, n=10 human clusters/mouse, n=1 mouse/vector. Statistical significances were calculated using the two Mann-Whitney test. P value <0.0001 (****); n.s.=non-significant (p value >0.05).

FIG. 7 is a graphical representation of the enrichment of capsid variants in a permutational AAV library following in vitro or in vivo selection. The data shows the percentage of NGS reads reflecting the prototypic AAV2 amino acid residue at a given position in the capsid polypeptide, or the “mutant” amino acid residue at a given position in the capsid polypeptide (e.g. where C is the “AAV2” residue at position 482, and S is the “mutant” residue at position 482). (A) Percentage of NGS reads for each amino acid at each amino acid position in the plasmid library prior to vectorisation. (B) Percentage of NGS reads for each amino acid at each amino acid position in the vector library prior to selection. (C) Percentage of NGS reads for each amino acid at each position in the vector library after three rounds of selection in HUH7 cells. (D) Percentage of NGS reads for each amino acid at each position in the vector library after two rounds of selection in hFRG mice.

FIG. 8 is a graphical representation of the in vivo transduction of hepatocytes by various AAV vectors. A naïve FRG mouse (without human hepatocytes) was injected with mix of AAV8 vector and two variants of AAV8 that contained residues that were known to increase binding to HSPG, AAV8_E533K and AAV8_RQNR. All three AAVs contained an eGFP expression cassette with unique barcodes. Mouse hepatocytes were recovered one week after AAV injection and next-generation sequencing (NGS) was performed on the DNA extracted from the cells. DNA specific for the barcodes and thus each vector was then quantified and is shown here as percentage of total NGS reads. NGS of the preinjection mix of the three AAVs was also performed for normalisation purposes. The initial vector mix was also deep sequenced for validation as shown.

FIG. 9 is a graphical representation of the in vitro transduction of HuH-7 cells by various AAV vectors. HuH-7 cells were transduced with AAV8 or AAV8_RQNR vectors at various MOIs, and transduction was assessed by detecting the percentage of GFP-positive cells.

FIG. 10 is a graphical representation of the in vivo transduction of an hFRG mouse by various AAV vectors. One hFRG mouse injected with each of the AAV vectors (5 barcoded transgenes/capsid). One week after injection the chimeric liver of the mouse was perfused and human and murine hepatocytes were single cell sorted. DNA was recovered from the populations of hepatocytes and Next Generation Sequencing (NGS) of the barcoded transgene in each of the AAV vectors was performed. DNA specific for the barcodes, and thus each vector, was then quantified. NGS reads from the DNA populations were normalized to the reads from the pre-injection mix.

FIG. 11 is a graphical representation of the in vivo transduction of hFRG mice by various AAV vectors. Two hFRG mice were injected with each of the AAV vectors (5 barcoded transgenes/capsid). One week after injection the chimeric liver of the mouse was perfused and human and murine hepatocytes were single cell sorted. DNA was recovered from the populations of hepatocytes and Next Generation Sequencing (NGS) of the barcoded transgene in each of the AAV vectors was performed. DNA and RNA specific for the barcodes, and thus each vector, was then quantified. NGS reads from the DNA and cDNA populations were normalized to the reads from the pre-injection mix. (A) DNA specific for the vectors in human hepatocytes. (B) cDNA (RNA) specific for the vectors in human hepatocytes. (C) Immunohistochemical analysis of hFRG mouse liver transduced with indicated the AAV vectors. Quantification of the percentage of transduced human hepatocytes per human cluster. Data shown as mean+SD, n=10 human clusters/mouse.

FIG. 12 is a graphical representation of the in vitro transduction of HuH7 cells by various AAV vectors. HuH7 cells were transduced with vectors at an MOI of 50,000 in the presence or absence of 400 μg/mL heparin. (A) Transduction as assessed by determining the percentage of eGFP-positive cells. (B) Transduction as assessed by determining the mean fluorescence intensity (MFI).

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, a “vector” includes reference to both polynucleotide vectors and viral vectors, each of which are capable of delivering a transgene contained within the vector into a host cell. Vectors can be episomal, i.e., do not integrate into the genome of a host cell, or can integrate into the host cell genome. The vectors may also be replication competent or replication-deficient. Exemplary polynucleotide vectors include, but are not limited to, plasmids, cosmids and transposons. Exemplary viral vectors include, for example, AAV, lentiviral, retroviral, adenoviral, herpes viral and hepatitis viral vectors.

As used herein, “adeno-associated viral vector” or AAV vector refers to a vector in which the capsid is derived from an adeno-associated virus, including without limitation, AAV1, AAV2, AAV3, AAV4, AAV5, AAV6, AAV7, AAV8, AAV9, AAV10, AAV11, AAV12 or AAV13, AAV from other clades or isolates, or is derived from synthetic or modified AAV capsid proteins, including chimeric capsid proteins. In particular embodiments, the AAV vector has a capsid comprising a capsid polypeptide of the present disclosure. When referring to AAV vectors, both the source of the genome and the source of the capsid can be identified, where the source of the genome is the first number designated and the source of the capsid is the second number designated. Thus, for example, a vector in which both the capsid and genome are derived from AAV2 is more accurately referred to as AAV2/2. A vector with an AAV6-derived capsid and an AAV2-derived genome is most accurately referred to as AAV2/6. A vector with the synthetic DJ capsid and an AAV2-derived genome is most accurately referred to as AAV2/DJ. For simplicity, and because most vectors use an AAV2-derived genome, it is understood that reference to an AAV6 vector generally refers to an AAV2/6 vector, reference to an AAV2 vector generally refers to an AAV2/2 vector, etc. An AAV vector may also be referred to herein as “recombinant AAV”, “rAAV”, “recombinant AAV virion”, and “rAAV virion,” terms which are used interchangeably and refer to a replication-defective virus that includes an AAV capsid shell encapsidating an AAV genome. The AAV vector genome (also referred to as vector genome, recombinant AAV genome or rAAV genome) comprises a transgene flanked on both sides by functional AAV ITRs. Typically, one or more of the wild-type AAV genes have been deleted from the genome in whole or part, preferably the rep and/or cap genes. Functional ITR sequences are necessary for the rescue, replication and packaging of the vector genome into the rAAV virion.

The term “ITR” refers to an inverted terminal repeat at either end of the AAV genome. This sequence can form hairpin structures and is involved in AAV DNA replication and rescue, or excision, from prokaryotic plasmids. ITRs for use in the present disclosure need not be the wild-type nucleotide sequences, and may be altered, e.g., by the insertion, deletion or substitution of nucleotides, as long as the sequences provide for functional rescue, replication and packaging of rAAV.

As used herein, “functional” with reference to a capsid polypeptide means that the polypeptide can self-assemble or assemble with different capsid polypeptides to produce the proteinaceous shell (capsid) of an AAV virion. It is to be understood that not all capsid polypeptides in a given host cell assemble into AAV capsids. Preferably, at least 25%, at least 50%, at least 75%, at least 85%, at least 90%, at least 95% of all AAV capsid polypeptide molecules assemble into AAV capsids. Suitable assays for measuring this biological activity are described e.g. in Smith-Arica and Bartlett (2001), Curr Cardiol Rep 3(1): 43-49.

“AAV helper functions” or “helper functions” refer to functions that allow AAV to be replicated and packaged by a host cell. AAV helper functions can be provided in any of a number of forms, including, but not limited to, as a helper virus or as helper virus genes which aid in AAV replication and packaging. Helper virus genes include, but are not limited to, adenoviral helper genes such as E1A, E1B, E2A, E4 and VA. Helper viruses include, but are not limited to, adenoviruses, herpesviruses, poxviruses such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

As used herein, “corresponding nucleotides” or “corresponding amino acid residues” refer to nucleotides or amino acids that occur at aligned loci. The sequences of related or variant polynucleotides or polypeptides are aligned by any method known to those of skill in the art. Such methods typically maximize matches (e.g. identical nucleotides or amino acids at positions), and include methods such as using manual alignments and by using the numerous alignment programs available (for example, BLASTN, BLASTP, ClustIW, ClustlW2, EMBOSS, LALIGN, Kalign, etc.) and others known to those of skill in the art. By aligning the sequences of polynucleotides or polypeptides, one skilled in the art can identify corresponding positions, nucleotides or amino acid residues. For example, by aligning the AAV capsid polypeptide set forth in SEQ ID NO:1 with another AAV capsid polypeptide, one of skill in the art can identify positions or amino acids residues within the other AAV polypeptide that correspond to positions or amino acid residues in the AAV polypeptide set forth in SEQ ID NO:1. For example, the methionine at position 204 of SEQ ID NO:1 is the corresponding amino acid of, or corresponds to, the methionine at position 203 of SEQ ID NO:55.

A “heterologous coding sequence” as used herein refers to nucleic acid sequence present in a polynucleotide, vector, or host cell that is not naturally found in the polynucleotide, vector, or host cell or is not naturally found at the position that it is at in the polynucleotide, vector, or host cell, i.e. is non-native. A “heterologous coding sequence” can encode a peptide or polypeptide, or a polynucleotide that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous sequence and the genomic DNA can occur. In one example, the heterologous coding sequence is a functional copy of a gene for introduction into a cell that has a defective/mutated copy.

As used herein, the term “operably-linked” with reference to a promoter and a coding sequence means that the transcription of the coding sequence is under the control of, or driven by, the promoter.

The term “heparin binding capacity” as used herein refers to the ability of a capsid polypeptide or AAV vector to bind to heparin. Heparin binding capacity can be assessed using, for example, heparin binding assays in which the capsid polypeptide or AAV vector is applied to an affinity chromatography medium to which heparin, heparan sulphate and/or heparan sulphate proteoglycan is attached. The strength to which the capsid polypeptide or AAV vector binds to the medium is then assessed, and may be represented as, for example, NaCl concentration or conductivity at peak elution. Other techniques can be used to assess heparin binding capacity. For example, the binding affinity of capsid polypeptide or AAV vector to heparin (as represented by the equilibrium dissociation constant K_{D}) can be assessed by surface plasmon resonance, e.g. using a BIAcore™ system. As would be appreciated, given that heparin is closely related to heparan sulphate and HSPG, the ability of an AAV vector or capsid polypeptide to bind to heparin is considered to be representative of the heparan sulphate and/or HSPG binding capacity of the AAV vector or capsid polypeptide. Thus, heparan sulphate and/or HSPG binding capacity can be assessed indirectly by measuring the ability of an AAV vector or capsid polypeptide to bind to. heparin.

The term “host cell” refers to a cell, such as a mammalian cell, that has introduced into it exogenous DNA, such as a vector or other polynucleotide. The term includes the progeny of the original cell into which the exogenous DNA has been introduced. Thus, a “host cell” as used herein generally refers to a cell that has been transfected or transduced with exogenous DNA.

As used herein, “isolated” with reference to a polynucleotide or polypeptide means that the polynucleotide or polypeptide is substantially free of cellular material or other contaminating proteins from the cells from which the polynucleotide or polypeptide is derived, or substantially free from chemical precursors or other chemicals when chemically synthesized.

As used herein, a “liver-associated disease or condition” is any disease or condition that can be treated by expression of a heterologous coding sequence in hepatocytes of the subject with the disease or condition.

The term “transduction efficiency” and grammatical variations thereof refers to the ability of an AAV vector to transduce host cells, and more particularly the efficiency with which an AAV vector transduces host cells. In particular embodiment, the transduction efficiency is in vivo transduction efficiency, and refers to the ability of an AAV vector to transduce host cells in vivo following administration of the vector to the subject. Transduction efficiency can be assessed in a number of ways known to those in the art, including assessing the number of host cells transduced following exposure to, or administration of, a given number of vector particles (e.g. as assessed by expression of a reporter gene from the vector genome, such as GFP or eGFP, using microscopy or flow cytometry techniques); the amount of vector DNA (e.g. number of vector genomes) in a population of host cells following exposure to a given number of vector particles; the amount of vector RNA in population of host cells following exposure to a given number of vector particles; and the level of protein expression from a reporter gene (e.g. GFP or eGFP) in the vector genome in a population of host cells following exposure to, or administration of, a given number of vector particles. The population of host cells can represent a particular number of host cells, a volume or weight of tissue, or an entire organ (e.g. liver). In vivo transduction efficiency can reflect the ability of an AAV vector to access host cells, such as hepatocytes in the liver; the ability of an AAV vector to enter host cells; and/or expression of a heterologous coding sequence contained in the vector genome upon host cell entry.

The phrase “suitable for in vivo transduction of human hepatocytes” means that the AAV vector has a transduction efficiency in human hepatocytes that results in sufficient cell entry of the vector, and/or sufficient expression in the cell of a heterologous coding sequence present in the vector genome, for the desired purpose, e.g. therapeutic effect. Typically, the transduction efficiency of an AAV vector that is suitable for in vivo transduction of human hepatocytes is at least or about 4-fold, 6-fold, 8-fold, 10-fold, 15-fold, 20-fold, 25-fold, 30-fold, 40-fold, 50-fold, 60-fold, 70-fold, 80-fold, 90-fold or 100-fold greater than an AAV vector comprising the prototypic AAV2 capsid set forth in SEQ ID NO:1.

The term “subject” as used herein refers to an animal, in particular a mammal and more particularly a primate including a lower primate and even more particularly, a human who can benefit from the present invention. A subject regardless of whether a human or non-human animal or embryo may be referred to as an individual, subject, animal, patient, host or recipient. The present disclosure has both human and veterinary applications. For convenience, an “animal” specifically includes livestock animals such as cattle, horses, sheep, pigs, camelids, goats and donkeys, as well as domestic animals, such as dogs and cats. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates. In some embodiments, the subject is human.

It will be appreciated that the above described terms and associated definitions are used for the purpose of explanation only and are not intended to be limiting.

TABLE 1
Brief Description of the Sequences

| SEQ ID NO. | Description |
|---|---|
| 1 | Prototypic AAV2 capsid polypeptide |
| 2 | CMRI_01 capsid polypeptide (VP1) |
| 3 | CMRI_02 capsid polypeptide (VP1) |
| 4 | CMRI_03 capsid polypeptide (VP1) |
| 5 | CMRI_04 capsid polypeptide (VP1) |
| 6 | CMRI_05 capsid polypeptide (VP1) |
| 7 | CMRI_06 capsid polypeptide (VP1) |
| 8 | CMRI_07 capsid polypeptide (VP1) |
| 9 | CMRI_08 capsid polypeptide (VP1) |
| 10 | CMRI_09 capsid polypeptide (VP1) |
| 11 | CMRI_10 capsid polypeptide (VP1) |
| 12 | CMRI_11 capsid polypeptide (VP1) |
| 13 | CMRI_12 capsid polypeptide (VP1) |
| 14 | CMRI_13 capsid polypeptide (VP1) |
| 15 | CMRI_14 capsid polypeptide (VP1) |
| 16 | CMRI_15 capsid polypeptide (VP1) |
| 17 | CMRI_16 capsid polypeptide (VP1) |
| 18 | CMRI_17 capsid polypeptide (VP1) |
| 19 | CMRI_18 capsid polypeptide (VP1) |
| 20 | CMRI_19 capsid polypeptide (VP1) |
| 21 | CMRI_20 capsid polypeptide (VP1) |
| 22 | CMRI_21 capsid polypeptide (VP1) |
| 23 | CMRI_22 capsid polypeptide (VP1) |
| 24 | CMRI_23 capsid polypeptide (VP1) |
| 25 | CMRI_24 capsid polypeptide (VP1) |
| 26 | CMRI_25 capsid polypeptide (VP1) |
| 27 | CMRI_26 capsid polypeptide (VP1) |
| 28 | CMRI_01 capsid polynucleotide |
| 29 | CMRI_02 capsid polynucleotide |
| 30 | CMRI_03 capsid polynucleotide |
| 31 | CMRI_04 capsid polynucleotide |
| 32 | CMRI_05 capsid polynucleotide |
| 33 | CMRI_06 capsid polynucleotide |
| 34 | CMRI_07 capsid polynucleotide |
| 35 | CMRI_08 capsid polynucleotide |
| 36 | CMRI_09 capsid polynucleotide |
| 37 | CMRI_10 capsid polynucleotide |
| 38 | CMRI_11 capsid polynucleotide |
| 39 | CMRI_12 capsid polynucleotide |
| 40 | CMRI_13 capsid polynucleotide |
| 41 | CMRI_14 capsid polynucleotide |
| 42 | CMRI_15 capsid polynucleotide |
| 43 | CMRI_16 capsid polynucleotide |
| 44 | CMRI_17 capsid polynucleotide |
| 45 | CMRI_18 capsid polynucleotide |
| 46 | CMRI_19 capsid polynucleotide |
| 47 | CMRI_20 capsid polynucleotide |
| 48 | CMRI_21 capsid polynucleotide |
| 49 | CMRI_22 capsid polynucleotide |
| 50 | CMRI_23 capsid polynucleotide |
| 51 | CMRI_24 capsid polynucleotide |
| 52 | CMRI_25 capsid polynucleotide |
| 53 | CMRI_26 capsid polynucleotide |
| 54 | Prototypic AAV2 capsid polynucleotide (cap) |
| 55 | Prototypic AAV13 capsid polypeptide |
| 56 | Prototypic AAV3 capsid polypeptide |
| 57 | Rep_mpx1_F primer |
| 58 | 3UTR_mpx2_R primer |
| 59 | AAV(excl5)rep_fwd primer |
| 60 | AAV(prim)3ITR-D + G_R primer |
| 61 | AAV(prim)3ITR-D + C_R primer |
| 62 | CMRI_27 capsid polypeptide |
| 63 | CMRI_28 capsid polypeptide |
| 64 | CMRI_29 capsid polypeptide |
| 65 | CMRI_30 capsid polypeptide |
| 66 | CMRI_27 capsid polynucleotide |
| 67 | CMRI_28 capsid polynucleotide |
| 68 | CMRI_29 capsid polynucleotide |
| 69 | CMRI_30 capsid polynucleotide |
| 70 | LK03 capsid polypeptide |
| 71 | CMRI_31 capsid polynucleotide |
| 72 | CMRI_32 capsid polynucleotide |
| 73 | CMRI_33 capsid polynucleotide |
| 74 | CMRI_34 capsid polynucleotide |
| 75 | Reverse primer cDNA_R |
| 76 | primer F_NGS |
| 77 | Primer R_NGS |

### 1. Methods for Producing AAV Vectors and Enhancing In Vivo Transduction Efficiency

The present disclosure is predicated in part on the determination that the in vivo transduction efficiency of AAV vectors is related to their ability to bind heparin, whereby AAV vectors that bind heparin too strongly typically exhibit inferior in vivo transduction efficiency compared to AAV vectors that bind heparin less strongly. Heparin is closely related to HSPG, which is present on the extracellular matrix of liver and other organs. Binding of AAV to heparin is reflective of the ability of the AAV to bind to HSPG. While not being bound by theory, it is postulated that HSPG molecules may be sequestering AAV vectors that bind strongly to HSPG, reducing the ability of the vectors to access and transduce the desired target cells, such as human hepatocytes in the liver. Consequently, reducing the binding affinity of an AAV vector for heparin, and thus for HSPG, can increase the in vivo transduction efficacy of the vector. This can be done, for example, by modifying the capsid polypeptide, e.g. by substituting one or more amino acid residues that may contribute to the binding of the AAV capsid to HSPG such that the affinity of the capsid polypeptide for heparin after modification is altered compared to its affinity before modification. Thus, in some aspects, the methods of the present disclosure are broadly directed to identifying (or selecting) a reference capsid polypeptide for in vivo transduction of human hepatocytes, then modifying that capsid polypeptide so as to reduce heparin binding affinity/capacity, thereby enhancing the in vivo human hepatocyte transduction efficiency of an AAV vector with a capsid comprising or consisting of the modified capsid polypeptide (e.g. an AAV vector produced by vectorising the modified capsid polypeptide) compared to an AAV vector with a capsid comprising or consisting of the reference capsid polypeptide (e.g. an AAV vector produced by vectorising the modified capsid polypeptide).

Assessment of the heparin binding capacity of AAV vectors using a heparin affinity chromatography medium is well known in the art. Typically, the AAV vector is applied to an affinity chromatography medium (e.g. sepharose, agarose, etc.) to which heparin, heparan sulphate and/or HSPG is attached. The AAV vector is applied under conditions suitable for binding of the vector to the medium, and then eluted with varying concentrations of salt, e.g. NaCl. In particular embodiments, a gradient of salt, e.g. NaCl, is applied to the column to elute the vector (e.g. a gradient of 0-100% NaCl). In representative embodiments, the chromatography medium is packed into a chromatography column and chromatography is carried out using gravity flow, HPLC (high performance liquid chromatography) or FPLC Fast protein liquid chromatography. The NaCl concentration and/or the conductivity at peak elution can then be determined. In exemplary embodiments, the chromatography medium is sepharose, to which heparin is attached, e.g. via the N-hydroxysuccinamide coupling method. The chromatography medium may be suitably packed into a column, e.g. at a volume of 1 mL. A suitable pre-packed column for assessing the heparin binding capacity of an AAV vector is the HiTrap® Heparin HP column (GE Healthcare). In illustrative embodiments, the NaCl concentration and/or the conductivity at peak elution is then determined by chromatography at a flow of, or about, 1 mL/minute.

The methods of can also include assessing the ability of the candidate AAV vector to transduce human hepatocytes in vivo. This can be done using small animal models, such as small animals (e.g. mouse, rat, guinea pig, rabbit, ferret, hamster, etc.) that have a chimeric liver that comprises human hepatocytes. Such models are known in the art and include, but are not limited to, albumin enhancer promoter-driven urokinase plasminogen activator transgenic mice (uPA-Tg mice) repopulated with human hepatocytes, Fah^{-/-}/Rag2^{-/-}/Il2rg^{-/-} (FRG) mice repopulated with human hepatocytes (hFRG mice), urokinase-type plasminogen activator/severe combined immunodeficiency (uPA/SCID) chimeric mice repopulated with human hepatocytes, hemizygous cDNA-uPA/SCID chimeric mice repopulated with human hepatocytes.

1.1. Modifying the Capsid Polypeptide

Provided are methods for producing modified AAV vectors, such as a modified AAV vectors for use in transducing human hepatocytes in vivo, and methods for enhancing the in vivo human hepatocyte transduction efficiency of an AAV vector, wherein the methods include identifying a reference capsid polypeptide (e.g. an AAV2 or AAV2-like capsid polypeptide) for transducing human hepatocytes in vivo, modifying the sequence of the reference capsid polypeptide so as to produce a modified capsid polypeptide (e.g. by modifying the sequence of a reference capsid polynucleotide encoding the reference capsid polypeptide so as to produce a modified capsid polynucleotide encoding a modified capsid polypeptide), and then vectorising the modified capsid polypeptide to produce the modified AAV vector, wherein the modified AAV vector is characterized in that it elutes from a heparin affinity chromatography medium with a NaCl concentration of less than, or no more than, about 400-450 mM NaCl, such as less than, or no more than, about 400, 405, 410, 415, 420, 425, 430, 435, 440, 445, or 450 mM; and/or with a conductivity of less than, or no more than, about 35-41 mS/cm, such as less than, or no more than, about 35, 35.5, 36, 36.5, 37, 37.5, 38, 38.5, 39, 39.5, 40, 40.5 or 41 mS/cm (e.g. when assessed by chromatography at a flow rate of 1 mL/min using a chromatography column packed with 1 mL sepharose to which heparin is immobilized (e.g. a HiTrap® Heparin HP column)). The AAV vector produced by these methods typically has an in vivo transduction efficiency that is enhanced compared to a reference AAV vector having a capsid comprising the reference capsid polypeptide. The transduction efficiency can be enhanced by at least or about, 10%, 20%, 30%, 40%, 50%, 60%, 70%, 80%, 90%, 100%, 200%, 300%, 400%, 500%, 600%, 700%, 800%, 900% 1,000%, or more, e.g. the transduction efficiency of the AAV vector can be at least or about 2×, 3×, 4×, 5×, 6×, 7×, 8×, 9×, 10×, 12×, 13×, 14×, 15×, 16×, 17×, 18×, 19×, 20×, 30×, 40×, 50×, 60×, 70×, 80×, 90×, 100× or more efficient at transducing cells in vivo.

At least one of the modifications made to the reference capsid polypeptide will alter the affinity, or binding capacity, of the polypeptide for heparin and/or HSPG. The binding of an AAV capsid to HSPG is mainly mediated by electrostatic interactions, whereby, in a simplification of the interaction, positive charges on the AAV capsid interact with negative charges on HSPG. Thus, in some instances, the AAV capsid polypeptide is modified to reduce the negative charge of the capsid, and in particular the negative charge of one or more amino acid residues or regions of the capsid that interacts with HSPG. Other modifications that do not necessarily affect the charge of an amino acid residue of an AAV capsid polypeptide can also, or can alternatively, be made to alter the ability of the capsid to bind to heparin/HSPG. Most typically, the ability of the AAV capsid polypeptide (or the ability of the AAV vector comprising the capsid polypeptide) to bind to heparin/HSPG is reduced compared to the reference capsid polypeptide (or the reference AAV vector comprising the reference capsid polypeptide), such as when the reference polypeptide comprises a sequence set forth in SEQ ID NO:1.

Exemplary modifications that can be made to a reference AAV capsid polypeptide (or polynucleotide, e.g. a polynucleotide encoding an AAV2 or AAV2-like capsid polypeptide) in the methods of the present disclosure include those resulting in an amino acid substitution in the encoded polypeptide of any one or more of the amino acid residues at positions corresponding to positions 482, 484, 487, 496, 503, 532, 582, 585, 588, 589 and 596 of the prototypic AAV2 capsid polypeptide set forth in SEQ ID NO:1. Amino acid substitutions at 1, 2, 3, 4, 5, 6, 7, 8, 9, 10 or all 11 positions corresponding to positions 482, 484, 487, 496, 503, 532, 582, 585, 588, 589 and 596 of SEQ ID NO:1 may be made. The amino acid residue at any one or more of these positions may be substituted with any another amino acid residue. In particular examples, positions corresponding to positions 482 and 496; 482 and 532; 482, 496 and 532; 482 and 585; 482, 503 and 596; 496, 588 and 596; 496, 532, 585, 588 and 596; or 503, 585, 588 and 596, are modified, such as by amino acid substitution.

In particular examples, the modifications include those resulting in an amino acid substitution in the encoded polypeptide of any one or more of the amino acid residues at positions corresponding to positions 482, 484, 487, 496, 503, 532, 582, 585, 588, 589 and 596 of SEQ ID NO:1. Exemplary substitutions include those resulting in a serine (S) or threonine (T) at the position corresponding to position 482 of SEQ ID NO:1 (e.g. C482S or C482T relative to SEQ ID NO:1); those resulting in a glutamine (Q) or asparagine (N) at position 484 (e.g. R484Q or R484N relative to SEQ ID NO:1); those resulting in a glutamine (Q) or asparagine (N) at position 487 (e.g. R487Q or R487N relative to SEQ ID NO:1); those resulting in an aspartic acid (D) or glutamic acid (E) at the position corresponding to position 496 of SEQ ID NO:1 (e.g. N496D or N496E relative to SEQ ID NO:1); those resulting in an alanine (A), valine (V) or isoleucine (I) at the position corresponding to position 503 of SEQ ID NO:1 (e.g. T503A, T503V, or T503I relative to SEQ ID NO:1); those resulting in a glutamic acid (E) or glutamine (Q) at the position corresponding to position 532 of SEQ ID NO:1 (e.g. K532E or K532Q relative to SEQ ID NO:1); those resulting in a serine (S), aspartic acid (D), tyrosine (Y), or threonine (T) at the position corresponding to position 582 of SEQ ID NO:1 (e.g. N582S, N582D, N582Y or N582T relative to SEQ ID NO:1); those resulting in a serine (S), threonine (T), glycine (G), alanine (A) or glutamic acid (E) at the position corresponding to position 585 of SEQ ID NO:1 (e.g. R585S, R585T, R585G, R585A or R585E, relative to SEQ ID NO:1); those resulting in a threonine (T), isoleucine (I) or alanine (A) at the position corresponding to position 588 of SEQ ID NO:1 (e.g. R588T, R588I or R588A relative to SEQ ID NO:1); and those resulting in an aspartic acid (D), glutamic acid (E) or alanine (A) at the position corresponding to position 589 of SEQ ID NO:1 (e.g. Q589D, Q589E or Q589A relative to SEQ ID NO:1); and those resulting in an aspartic acid (D) or glutamic acid (E) at the position corresponding to position 596 of SEQ ID NO:1 (e.g. N596D or N596E relative to SEQ ID NO:1).

In particular examples, two or more of the above modifications are made, and include those resulting in a serine (S) at the position corresponding to position 482 and an aspartic acid (D) at the position corresponding to position 496 (e.g. C482S and N496D relative to SEQ ID NO:1); a serine (S) at the position corresponding to position 482 and a glutamine (Q) at the position corresponding to position 532 (e.g. C482S and K532Q); a serine (S) at the position corresponding to position 482, an aspartic acid (D) at the position corresponding to position 496 and a glutamine (Q) at the position corresponding to position 532 (e.g. C482S, N496D and K532Q relative to SEQ ID NO:1); a serine (S) at the position corresponding to position 482 and a serine (S) at the position corresponding to position 585 (e.g. C482S and R585S relative to SEQ ID NO:1); a serine (S) at the position corresponding to position 482, an alanine (A) at the position corresponding to position 503 and an aspartic acid (D) at the position corresponding to position 596 (e.g. C482S, T503A and N596D relative to SEQ ID NO:1); an alanine (A) at the position corresponding to position 503 and an aspartic acid (D) at the position corresponding to position 596 (e.g. T503A and N596D relative to SEQ ID NO:1); an aspartic acid (D) at the position corresponding to position 496, a glutamine (Q) at the position corresponding to position 532, a serine (S) at the position corresponding to position 585, a threonine (T) at the position corresponding to position 588 and an aspartic acid (D) at the position corresponding to position 596 (e.g. N496D, K532Q, R585S, R588T and N596D relative to SEQ ID NO:1); an aspartic acid (D) at the position corresponding to position 496, a threonine (T) at the position corresponding to position 588 and an aspartic acid (D) at the position corresponding to position 596 (e.g. N496D, R588T and N596D relative to SEQ ID NO:1); an alanine (A) at the position corresponding to position 503, a serine (S) at the position corresponding to position 585, a threonine (T) at the position corresponding to position 588 and an aspartic acid (D) at the position corresponding to position 596 (e.g. T503A, R585S, R588T and N596D relative to SEQ ID NO:1).

Also provided are methods for producing modified AAV vectors (such as a modified AAV vector for use in transducing human hepatocytes in vivo) and methods for enhancing the transduction efficiency of an AAV vector, wherein the methods include identifying a reference capsid polypeptide (e.g. an AAV3B or AAV3B-like capsid polypeptide) for transducing human hepatocytes in vivo, modifying the sequence of the reference capsid polypeptide so as to produce a modified capsid polypeptide (e.g. by modifying the sequence of a reference capsid polynucleotide encoding the reference capsid polypeptide so as to produce a modified capsid polynucleotide encoding a modified capsid polypeptide), and then vectorising the modified capsid polypeptide to produce the modified AAV vector, wherein the modified AAV vector is characterized in that it elutes from a heparin affinity chromatography medium with a NaCl concentration of less than, or no more than, about 225-277 mM NaCl, such as less than, or no more than, about 225, 230, 235, 240, 245, 250, 255, 260, 265, 270, 275, or 277 mM; and/or with a conductivity of less than, or no more than, about 20-26 mS/cm, such as less than, or no more than, about 20, 20.5, 21, 21.5, 22, 22.5, 23, 23.5, 24, 24.5, 25, 25.5 or 26 mS/cm (e.g. when assessed by chromatography at a flow rate of 1 mL/min using a chromatography column packed with 1 mL sepharose to which heparin is immobilized (e.g. a HiTrap® Heparin HP column)). The AAV vector produced by these methods typically has an in vivo transduction efficiency that is enhanced compared to a reference AAV vector having a capsid comprising the reference capsid polypeptide. The transduction efficiency can be enhanced by at least or about, 10%, 20%, 30%, 40%, 50%, 60%, 70%, 80%, 90%, 100%, 200%, 300%, 400%, 500%, 600%, 700%, 800%, 900% 1,000%, or more, e.g. the transduction efficiency of the AAV vector can be at least or about 2×, 3×, 4×, 5×, 6×, 7×, 8×, 9×, 10×, 12×, 13×, 14×, 15×, 16×, 17×, 18×, 19×, 20×, 30×, 40×, 50×, 60×, 70×, 80×, 90×, 100× or more efficient at transducing cells in vivo.

Exemplary modifications that can be made to a reference AAV capsid polypeptide (or polynucleotide, e.g. a polynucleotide encoding an AAV3B or AAV3B-like capsid polypeptide) in the methods of the present disclosure include those resulting in an amino acid substitution in the encoded polypeptide of any one or more of the amino acid residues at positions corresponding to positions 476, 485, 488, 533, 594 and 598 of the prototypic AAV3B capsid polypeptide set forth in SEQ ID NO:56). Amino acid substitutions at 1, 2, 3, 4, 5 or 6 positions corresponding to positions 476, 485, 488, 533, 594 and 598 of SEQ ID NO:56 may be made. In one example, substitutions at positions corresponding to positions 594 and 598 of SEQ ID NO:56 are made. The amino acid residue at any one or more of these positions may be substituted with any another amino acid residue. Exemplary substitutions include those resulting in a glutamine (Q) or asparagine (N) at position 476 (e.g. R476Q or R476N relative to SEQ ID NO:56); those resulting in a glutamine (Q) or asparagine (N) at position 485 (e.g. R485Q or R485N relative to SEQ ID NO:56); those resulting in a glutamine (Q) or asparagine (N) at position 488 (e.g. R488Q or R488N relative to SEQ ID NO:56); those resulting in a glutamic acid (E) or glutamine (Q) at position 533 (e.g. K533E or K533Q relative to SEQ ID NO:56); those resulting in a glutamic acid (E) or histidine (H) at position 598 (e.g. D598E or D598H relative to SEQ ID NO:56); and those resulting in a glycine (G), glutamic acid (E) or alanine (A) at position 594 (e.g. R594G, R594E or R594A relative to SEQ ID NO:56). In one embodiment, the two modifications are made, with one resulting in glutamic acid (E) at position 594 (e.g. R594E) and one resulting in histidine (H) at position 598 (e.g. D598H).

Also provided are methods for producing modified AAV vectors, such as a modified AAV vectors for use in transducing human hepatocytes in vivo, and methods for enhancing the transduction efficiency of an AAV vector, wherein the methods include identifying a reference capsid polypeptide (e.g. an AAV13 or AAV13-like capsid polypeptide) for transducing human hepatocytes in vivo, modifying the sequence of the reference capsid polypeptide so as to produce a modified capsid polypeptide (e.g. by modifying the sequence of a reference capsid polynucleotide encoding the reference capsid polypeptide so as to produce a modified capsid polynucleotide encoding a modified capsid polypeptide), and then vectorising the modified capsid polypeptide to produce the modified AAV vector, wherein the modified AAV vector is characterized in that it elutes from a heparin affinity chromatography medium with a NaCl concentration of less than, or no more than, about 280-330 mM NaCl, such as less than, or no more than, about 280, 285, 290, 295, 300, 305, 310, 315, 320, 325 or 330 mM; and/or with a conductivity of less than, or no more than, about 24-30 mS/cm, such as less than, or no more than, about 24, 24.5, 25, 25.5, 26, 26.5, 27, 27.5, 28, 28.5, 29, 29.5 or 30 mS/cm (e.g. when assessed by chromatography at a flow rate of 1 mL/min using a chromatography column packed with 1 mL sepharose to which heparin is immobilized (e.g. a HiTrap® Heparin HP column)). The AAV vector produced by these methods typically has an in vivo transduction efficiency that is enhanced compared to a reference AAV vector having a capsid comprising the reference capsid polypeptide. The transduction efficiency can be enhanced by at least or about, 10%, 20%, 30%, 40%, 50%, 60%, 70%, 80%, 90%, 100%, 200%, 300%, 400%, 500%, 600%, 700%, 800%, 900% 1,000%, or more, e.g. the transduction efficiency of the AAV vector can be at least or about 2×, 3×, 4×, 5×, 6×, 7×, 8×, 9×, 10×, 12×, 13×, 14×, 15×, 16×, 17×, 18×, 19×, 20×, 30×, 40×, 50×, 60×, 70×, 80×, 90×, 100× or more efficient at transducing cells in vivo.

Exemplary modifications that can be made to a reference AAV capsid polypeptide (or polynucleotide, e.g. a polynucleotide encoding an AAV13 or AAV13-like capsid polypeptide) in the methods of the present disclosure include those resulting in an amino acid substitution in the encoded polypeptide of any one or more of the amino acid residues at positions corresponding to positions 482, 485, 525, 528 and 530 of the prototypic AAV13 capsid polypeptide set forth in SEQ ID NO:55. The amino acid residue at any one or more of these positions may be substituted with any another amino acid residue. As discussed above, an AAV vector comprising such a capsid polypeptide typically exhibits improved in vivo transduction efficiency compared to an AAV vector comprising a prototypic AAV13 capsid polypeptide. Exemplary substitutions include those resulting in a glutamine (Q) or asparagine (N) at position 482 (e.g. R482Q or R482N relative to SEQ ID NO:55); those resulting in a glutamine (Q) or asparagine (N) at position 485 (e.g. R485Q or R485N relative to SEQ ID NO:55); those resulting in a glutamic acid (E) or aspartic acid (D) at position 525 (e.g. K525E or K525Q relative to SEQ ID NO:55); those resulting in a glutamic acid (E) or aspartic acid (D) at position 528 (e.g. K528E or K528Q relative to SEQ ID NO:55); and those resulting in a glutamic acid (E) or aspartic acid (D) at position 530 (e.g. K530E or K530Q relative to SEQ ID NO:55).

The reference capsid polypeptide may be any AAV polypeptide, such as an AAV1, AAV2, AAV3, AAV3B, AAV4, AAV5, AAV6, AAV7, AAV8, AAV9, AAV10, AAV11, AAV12 or AAV13 capsid polypeptide, or a synthetic or chimeric capsid polypeptide. In illustrative embodiments, the reference polypeptide is an AAV2 (or AAV2-like), AAV3B (or AAV3B-like) or AAV13 (or AAV13-like) capsid polypeptide. In particular examples, the reference polypeptide is an AAV2 or AAV2-like polypeptide, such as one set forth in SEQ ID NO:1 or a polypeptide having at least or about 85%, 86%, 87%, 88%, 89%, 90%, 91%, 92%, 93%, 94%, 95%, 96%, 97%, 98%, or 99% sequence identity to the sequence set forth in SEQ ID NO:1. Thus, for example, the reference capsid polypeptide may comprise a sequence of amino acids set forth in SEQ ID NO:1, and the one or more substitutions that are introduced into the sequence of the reference capsid polypeptide so as to produce the modified capsid polypeptide include one or more of the amino acid substitutions selected from among C482S, C482T, R484Q, R484N, R487Q, R487N, N496D, N496E, T503A, T503V, T503I, K532E, K532Q, N582S, N582D, N582Y, N582T, R585S, R585T, R585G, R585A, R585E, R588T, R588I, R588A, Q589D, Q589E, Q589A, N596D and N596E. In other examples, the reference capsid polypeptide comprises a sequence of amino acids having at least or about 90% or 95% sequence identity to the polypeptide set forth in SEQ ID NO:1. In further examples, the reference polypeptide is an AAV3B or AAV3B-like polypeptide, such as a polypeptide set forth in SEQ ID NO:56 or a polypeptide having at least or about 85%, 86%, 87%, 88%, 89%, 90%, 91%, 92%, 93%, 94%, 95%, 96%, 97%, 98%, or 99% sequence identity to the sequence set forth in SEQ ID NO:56. Thus, for example, the reference capsid polypeptide may comprise a sequence of amino acids set forth in SEQ ID NO:56, and the one or more substitutions that are introduced into the sequence of the reference capsid polypeptide so as to produce the modified capsid polypeptide include one or more of the amino acid substitutions selected from among R476Q, R476N, R485Q, R485N, R488Q, R488N, K533E, K533Q, D598E, D598H, R594G, R594E and R594A. In still further examples, the reference polypeptide is an AAV13 or AAV13-like polypeptide, such as a polypeptide set forth in SEQ ID NO:55 or a polypeptide having at least or about 85%, 86%, 87%, 88%, 89%, 90%, 91%, 92%, 93%, 94%, 95%, 96%, 97%, 98%, or 99% sequence identity to the sequence set forth in SEQ ID NO:55. Thus, for example, the reference capsid polypeptide may comprise a sequence of amino acids set forth in SEQ ID NO:55, and the one or more substitutions that are introduced into the sequence of the reference capsid polypeptide so as to produce the modified capsid polypeptide include one or more of the amino acid substitutions selected from among R482Q, R482N, R485Q, R485N, K525E, K525Q, K528E, K528Q K530E and K530Q.

Reference capsid polypeptides include those comprising all or a portion of the VP1 protein (comprising amino acid residues corresponding to those at positions 1-735 of SEQ ID NO:1, positions 1-736 of SEQ ID NO:56, or positions 1-733 of SEQ ID NO:55), VP2 protein (comprising amino acid residues corresponding to those at positions 138-735 of SEQ ID NO:1, positions 138-736 of SEQ ID NO:56, or positions 137-733 of SEQ ID NO:55) and/or the VP3 protein (comprising amino acid residues corresponding to those at positions 203-735 of SEQ ID NO:1, positions 203-736 of SEQ ID NO:56, or positions 202-733 of SEQ ID NO:55), as well as functional fragments thereof. As would be appreciated, the modified AAV capsid polypeptides can therefore include those comprising all or a portion of the VP1 protein (comprising amino acid residues corresponding to those at positions 1-735 of SEQ ID NO:1, positions 1-736 of SEQ ID NO:56, or positions 1-733 of SEQ ID NO:55), VP2 protein (comprising amino acid residues corresponding to those at positions 138-735 of SEQ ID NO:1 positions 138-736 of SEQ ID NO:56, or positions 137-733 of SEQ ID NO:55) and/or the VP3 protein (comprising amino acid residues corresponding to those at positions 203-735 of SEQ ID NO:1, positions 203-736 of SEQ ID NO:56, or positions 202-733 of SEQ ID NO:55) or functional fragments thereof.

Methods for modifying the sequence of a reference capsid polypeptide or polynucleotide so as to produce a modified capsid polypeptide or polynucleotide are well known in the art, and any such method can be utilised so as to perform the methods of the present disclosure. For example, the modification of the sequence of the reference capsid polynucleotide to produce a modified capsid polynucleotide can be performed using any method known in the art, including recombinant and synthetic methods, performed (either in part or in whole) in silico and/or in vitro. In a particular example, the modification of the sequence is performed in silico, followed by de novo synthesis of the modified capsid polynucleotide having the modified sequence (e.g. by gene synthesis methods such as those involving the chemical synthesis of overlapping oligonucleotides following by gene assembly).

The modified capsid polynucleotides may be contained in nucleic acid vector, such as a plasmid, for subsequent expression, replication, amplification and/or manipulation. Vectors suitable for use in bacterial, insect and mammalian cells are widely described and well-known in the art. Those skilled in the art would appreciate that the vectors may also contain additional sequences and elements useful for the replication of the vector in prokaryotic and/or eukaryotic cells, selection of the vector and the expression of a heterologous sequence in a variety of host cells. For example, the vectors can include a prokaryotic replicon, which is a sequence having the ability to direct autonomous replication and maintenance of the vector extrachromosomally in a prokaryotic host cell, such as a bacterial host cell. Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors can be constructed using known techniques, including, without limitation, the standard techniques of restriction endonuclease digestion, ligation, transformation, plasmid purification, in vitro or chemical synthesis of DNA, and DNA sequencing. The vectors comprising a modified capsid polynucleotide may be introduced into a host cell using any method known in the art.

1.2. Vectorising Capsid Polypeptides

Methods for vectorising a capsid polypeptide are well known in the art and any suitable method can be employed for the purposes of the present disclosure. For example, the modified capsid polynucleotide can be recovered (e.g. by PCR or digestion with restriction enzymes) and cloned into a packaging construct containing rep. Any AAV rep gene may be used, including, for example, a rep gene from AAV1, AAV2, AAV3, AAV4, AAV5, AAV6, AAV7, AAV8, AAV9, AAV10, AAV11, AAV12 or AAV13 and any variants thereof. Typically, the modified capsid polynucleotide is cloned downstream of rep so the rep p40 promoter can drive modified capsid polynucleotide expression, however constructs/system that utilise ubiquitous or tissue specific promoters to drive cap gene expression have been described. This construct does not contain ITRs. This construct is then introduced into a packaging cell line with a second construct containing ITRs, typically flanking a heterologous coding sequence. Helper elements/functions, or a helper virus, are also introduced, and recombinant AAV comprising a capsid generated from capsid proteins expressed from the modified capsid polynucleotide sequence, and encapsidating a genome comprising the transgene flanked by the ITRs, is recovered from the supernatant of the packaging cell line and/or from inside the packaging cells. Various types of cells can be used as the packaging cell line. For example, packaging cell lines that can be used include, but are not limited to, HEK 293 cells, HeLa cells, and Vero cells, for example as disclosed in US20110201088. The helper functions may be provided by one or more helper plasmids or helper viruses. Non-limiting examples of the adenoviral helper genes include E1A, E1B, E2A, E4 and VA, which can provide helper functions to AAV packaging. Helper viruses of AAV are known in the art and include, for example, viruses from the Adenoviridae family and the Herpesviridae family. Examples of helper viruses of AAV include, but are not limited to, SAdV-13 helper virus and SAdV-13-like helper virus described in US20110201088, helper vectors pHELP (Applied Viromics). A skilled artisan will appreciate that any helper virus or helper plasmid of AAV that can provide adequate helper function to AAV can be used herein.

In some instances, rAAV virions are produced using a cell line that stably expresses some of the necessary components for AAV virion production. For example, a plasmid (or multiple plasmids) comprising the nucleic acid containing a cap gene identified as described herein and a rep gene, and a selectable marker, such as a neomycin resistance gene, can be integrated into the genome of a cell (the packaging cells). The packaging cell line can then be transfected with an AAV vector and a helper plasmid or transfected with an AAV vector and co-infected with a helper virus (e.g., adenovirus providing the helper functions). The advantages of this method are that the cells are selectable and are suitable for large-scale production of the recombinant AAV. As another non-limiting example, adenovirus or baculovirus rather than plasmids can be used to introduce the nucleic acid encoding the capsid polypeptide, and optionally the rep gene, into packaging cells. As yet another non-limiting example, the AAV vector is also stably integrated into the DNA of producer cells, and the helper functions can be provided by a wild-type adenovirus to produce the recombinant AAV. In still further instances, the AAV vectors are produced synthetically, by synthesising AAV capsid proteins and assembling and packaging the capsids in vitro.

Thus, also provided are AAV vectors produced by the methods of the present disclosure.

1.3. Heterologous Coding Sequences and Other Elements

In some examples, the AAV vectors also comprise a heterologous coding sequence. The heterologous coding sequence may be operably linked to a promoter to facilitate expression of the sequence. The heterologous coding sequence can encode a peptide or polypeptide, such as a reporter protein (e.g. GFP or eGFP) or a therapeutic peptide or polypeptide, or can encode a polynucleotide or transcript that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous coding sequence and the genomic DNA can occur. As would be appreciated, the nature of the heterologous coding sequence is not essential to the present disclosure. In particular embodiments, the vectors comprising the heterologous coding sequence(s) will be used in gene therapy.

In particular examples, the heterologous coding sequence encodes a peptide or polypeptide, or polynucleotide, whose expression is of therapeutic use, such as, for example, for the treatment of a disease or disorder. For example, expression of a therapeutic peptide or polypeptide may serve to restore or replace the function of the endogenous form of the peptide or polypeptide that is defective (i.e. gene replacement therapy). In other examples, expression of a therapeutic peptide or polypeptide, or polynucleotide, from the heterologous sequence serves to alter the levels and/or activity of one or more other peptides, polypeptides or polynucleotides in the host cell. Thus, according to particular embodiments, the expression of a heterologous coding sequence introduced by a vector described herein into a host cell can be used to provide a therapeutic amount of a peptide, polypeptide or polynucleotide to ameliorate the symptoms of a disease or disorder. In other instances, the heterologous coding sequence is a stretch of nucleic acids that is essentially homologous to a stretch of nucleic acids in the genomic DNA of an animal, such that when the heterologous sequence is introduced into a cell of the animal, homologous recombination between the heterologous coding sequence and the genomic DNA can occur. Accordingly, the introduction of a heterologous sequence by an AAV vector described herein into a host cell can be used to correct mutations in genomic DNA, which in turn can ameliorate the symptoms of a disease or disorder.

The heterologous coding sequence in the AAV vector is flanked by the 3′ and 5′ AAV ITRs. AAV ITRs used in the vectors of the disclosure need not have a wild-type nucleotide sequence, and may be altered, e.g., by the insertion, deletion or substitution of nucleotides. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12 or AAV13. Such ITRs are well known in the art.

The nucleic acid vectors (e.g. plasmids, cosmids, etc.) comprising the modified capsid polynucleotides and the AAV vectors produced by the methods of the disclosure can comprise promoters. For example, a nucleic acid vector comprising a modified capsid polynucleotides may include a promoter that facilitates expression of the modified capsid polynucleotide. An AAV vector produced by the methods of the disclosure typically will comprise, in its vector genome, a promoter that facilitates expression of a heterologous coding sequence, as described above.

In some examples, the promoters are AAV promoters, such as the p5, p19 or p40 promoter. In other examples, the promoters are derived from other sources. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In some embodiments, tissue specific promoters are used. Non-limiting examples of such promoters include the liver-specific thyroxin binding globulin (TBG) promoter, insulin promoter, glucagon promoter, somatostatin promoter, pancreatic polypeptide (PPY) promoter, synapsin-1 (Syn) promoter, creatine kinase (MCK) promoter, mammalian desmin (DES) promoter, a α-myosin heavy chain (a-MHC) promoter, a cardiac Troponin T (cTnT) promoter, beta-actin promoter, and hepatitis B virus core promoter. The selection of an appropriate promoter is well within the ability of one of ordinary skill in the art.

The vectors can also include transcriptional enhancers, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly(A) sequences, and any variants thereof. In some embodiments, the transcriptional termination region is located downstream of the posttranscriptional regulatory element. In some embodiments, the transcriptional termination region is a polyadenylation signal sequence.

The vectors can include various posttranscriptional regulatory elements. In some embodiments, the posttranscriptional regulatory element can be a viral posttranscriptional regulatory element. Non-limiting examples of viral posttranscriptional regulatory element include woodchuck hepatitis virus posttranscriptional regulatory element (WPRE), hepatitis B virus posttranscriptional regulatory element (HBVPRE), RNA transport element, and any variants thereof. The RTE can be a rev response element (RRE), for example, a lentiviral RRE. A non-limiting example is bovine immunodeficiency virus rev response element (RRE). In some embodiments, the RTE is a constitutive transport element (CTE). Examples of CTE include, but are not limited, to Mason-Pfizer Monkey Virus CTE and Avian Leukemia Virus CTE.

A signal peptide sequence can also be included in the vector to provide for secretion of a polypeptide from a mammalian cell. Examples of signal peptides include, but are not limited to, the endogenous signal peptide for HGH and variants thereof; the endogenous signal peptide for interferons and variants thereof, including the signal peptide of type I, II and III interferons and variants thereof; and the endogenous signal peptides for known cytokines and variants thereof, such as the signal peptide of erythropoietin (EPO), insulin, TGF-β1, TNF, IL1-α, and IL1-β, and variants thereof. Typically, the nucleotide sequence of the signal peptide is located immediately upstream of the heterologous sequence (e.g., fused at the 5′ of the coding region of the protein of interest) in the vector.

In further examples, the vectors can contain a regulatory sequence that allows, for example, the translation of multiple proteins from a single mRNA. Non-limiting examples of such regulatory sequences include internal ribosome entry site (IRES) and 2A self-processing sequence, such as a 2A peptide site from foot-and-mouth disease virus (F2A sequence).

### 2. Methods of Screening and Identifying AAV Vectors

The determination that the heparin binding affinity of an AAV vector is related to its in vivo transduction efficiency can be exploited in methods for screening AAV vectors to identify those likely to be suitable for in vivo transduction of human hepatocytes. Screening candidate AAV vectors for their ability to bind heparin prior to performing in vivo studies can be an effective means for more rapidly, and more cheaply, identifying vectors that are likely to be effective gene delivery vehicles in vivo.

Thus, provided is a method for identifying a candidate AAV vector that is suitable for in vivo transduction of human hepatocytes, comprising assessing the heparin binding capacity of a test AAV vector and comparing the heparin binding capacity of the test AAV vector to the heparin binding capacity of a reference AAV vector (e.g. a prototypic AAV2 vector), wherein the test AAV vector is an AAV2 or AAV2-like vector comprising a capsid polypeptide comprising the sequence set forth in SEQ ID NO:1 or a sequence having at least or about 90%, 91%, 92%, 93%, 94%, 95%, 96%, 97%, 98% or 99% sequence identity thereto; and the reference AAV vector elutes from a heparin affinity chromatography medium with a NaCl concentration of at least 400-450 mM NaCl, such at least 400, 405, 410, 415, 420, 425, 430, 435, 440, 445, or 450 mM; and/or with a conductivity of at least 35-41 mS/cm, such as at least 35, 35.5, 36, 36.5, 37, 37.5, 38, 38.5, 39, 39.5, 40, 40.5 or 41 mS/cm (e.g. when assessed by chromatography at a flow rate of 1 mL/min using a chromatography column packed with 1 mL sepharose to which heparin is immobilized (e.g. a HiTrap® Heparin HP column)). When the test AAV vector has a heparin binding capacity below or less than that of the reference AAV vector, the test AAV vector is identified as a candidate AAV vector suitable for in vivo transduction of human hepatocytes.

Thus, the method can involve assessing the heparin binding capacity of a test AAV vector, wherein the test AAV vector is an AAV2 or AAV2-like vector comprising a capsid polypeptide comprising the sequence set forth in SEQ ID NO:1 or a sequence having at least or about 90%, 91%, 92%, 93%, 94%, 95%, 96%, 97%, 98% or 99% sequence identity thereto. If the heparin binding capacity of the test AAV vector is less than that of a reference AAV vector that elutes from a heparin affinity chromatography medium with a NaCl concentration of at least 400-450 mM NaCl, such at least 400, 405, 410, 415, 420, 425, 430, 435, 440, 445, or 450 mM, and/or with a conductivity of at least 35-41 mS/cm, such as at least 35, 35.5, 36, 36.5, 37, 37.5, 38, 38.5, 39, 39.5, 40, 40.5 or 41 mS/cm, then test AAV vector is identified as a candidate AAV vector suitable for in vivo transduction of human hepatocytes.

Also contemplated is a method for identifying a candidate AAV vector that is suitable for in vivo transduction of human hepatocytes, comprising assessing the heparin binding capacity of a test AAV vector and comparing the heparin binding capacity of the test AAV vector to the heparin binding capacity of a reference AAV vector (e.g. a prototypic AAV3B vector), wherein the test AAV vector is an AAV3B or AAV3B-like vector comprising a capsid polypeptide comprising the sequence set forth in SEQ ID NO:56 or a sequence having at least or about 90%, 91%, 92%, 93%, 94%, 95%, 96%, 97%, 98% or 99% sequence identity thereto; and the reference AAV vector elutes from a heparin affinity chromatography medium with a NaCl concentration of at least 225-277 mM NaCl, such as at least 225, 230, 235, 240, 245, 250, 255, 260, 265, 270, 275, or 277 mM; and/or with a conductivity of at least 20-26 mS/cm, such as at least 20, 20.5, 21, 21.5, 22, 22.5, 23, 23.5, 24, 24.5, 25, 25.5 or 26 mS/cm (e.g. when assessed by chromatography at a flow rate of 1 mL/min using a chromatography column packed with 1 mL sepharose to which heparin is immobilized (e.g. a HiTrap® Heparin HP column)). When the test AAV vector has a heparin binding capacity below or less than that of the reference AAV vector, the test AAV vector is identified as a candidate AAV vector suitable for in vivo transduction of human hepatocytes.

Thus, the method can involve assessing the heparin binding capacity of a test AAV vector, wherein the test AAV vector is an AAV3B or AAV3B-like vector comprising a capsid polypeptide comprising the sequence set forth in SEQ ID NO:56 or a sequence having at least or about 90%, 91%, 92%, 93%, 94%, 95%, 96%, 97%, 98% or 99% sequence identity thereto. If the heparin binding capacity of the test AAV vector is less than that of a reference AAV vector that elutes from a heparin affinity chromatography medium with a NaCl concentration of at least 225-277 mM NaCl, such as at least 225, 230, 235, 240, 245, 250, 255, 260, 265, 270, 275, or 277 mM, and/or with a conductivity of at least 20-26 mS/cm, such as at least 20, 20.5, 21, 21.5, 22, 22.5, 23, 23.5, 24, 24.5, 25, 25.5 or 26 mS/cm, then test AAV vector is identified as a candidate AAV vector suitable for in vivo transduction of human hepatocytes.

Also provided is a method for identifying a candidate AAV vector that is suitable for in vivo transduction of human hepatocytes, comprising assessing the heparin binding capacity of a test AAV vector and comparing the heparin binding capacity of the test AAV vector to the heparin binding capacity of a reference AAV vector (e.g. a prototypic AAV13 vector), wherein the test AAV vector is an AAV13 or AAV13-like vector comprising a capsid polypeptide comprising the sequence set forth in SEQ ID NO:55 or a sequence having at least or about 90%, 91%, 92%, 93%, 94%, 95%, 96%, 97%, 98% or 99% sequence identity thereto; and the reference AAV vector elutes from a heparin affinity chromatography medium with a NaCl concentration of at least 280-330 mM NaCl, such as at least 280, 285, 290, 295, 300, 305, 310, 315, 320, 325 or 330 mM; and/or with a conductivity of at least 24-30 mS/cm, such as at least 24, 24.5, 25, 25.5, 26, 26.5, 27, 27.5, 28, 28.5, 29, 29.5 or 30 mS/cm (e.g. when assessed by chromatography at a flow rate of 1 mL/min using a chromatography column packed with 1 mL sepharose to which heparin is immobilized (e.g. a HiTrap® Heparin HP column)). When the test AAV vector has a heparin binding capacity below or less than that of the reference AAV vector, the test AAV vector is identified as a candidate AAV vector suitable for in vivo transduction of human hepatocytes.

Thus, the method can involve assessing the heparin binding capacity of a test AAV vector, wherein the test AAV vector is an AAV13 or AAV13-like vector comprising a capsid polypeptide comprising the sequence set forth in SEQ ID NO:55 or a sequence having at least or about 90%, 91%, 92%, 93%, 94%, 95%, 96%, 97%, 98% or 99% sequence identity thereto. If the heparin binding capacity of the test AAV vector is less than that of a reference AAV vector that elutes from a heparin affinity chromatography medium with a NaCl concentration of at least 280-330 mM NaCl, such as at least 280, 285, 290, 295, 300, 305, 310, 315, 320, 325 or 330 mM; and/or with a conductivity of at least 24-30 mS/cm, such as at least 24, 24.5, 25, 25.5, 26, 26.5, 27, 27.5, 28, 28.5, 29, 29.5 or 30 mS/cm, then test AAV vector is identified as a candidate AAV vector suitable for in vivo transduction of human hepatocytes.

The heparin binding capacity of an AAV vector (i.e. the ability of the AAV vector to bind to heparin) can be assessed by any means know to the skilled person. In some examples, the heparin binding capacity of an AAV vector is measured indirectly by assessing the heparin binding capacity of the capsid polypeptide present in the capsid of the AAV vector. In particular embodiments, the heparin binding capacity is assessed in a binding assay in which the AAV vector or capsid polypeptide is applied to an affinity chromatography medium to which heparin, heparan sulphate and/or HSPG is attached. Such assays and chromatography media are widely used in the art and are exemplified herein in the Examples.

As would be appreciated, the binding assays typically involve contacting the affinity chromatography medium (e.g. sepharose, agarose etc.) with a sample containing the AAV vector or capsid polypeptide under conditions suitable for binding of the vector or polypeptide to the medium; and eluting the bound AAV vector or capsid polypeptide from the chromatography medium. Elution can be performed by changing salt concentrations, pH, pI, charge and/or ionic strength. In representative embodiments, the chromatography medium is packed into a chromatography column, and the AAV vector is purified from the sample by affinity chromatography using a NaCl gradient for elution. Chromatography can be carried out using, for example, gravity flow protocols, spin protocols, by HPLC (high performance liquid chromatography) or FPLC (fast protein liquid chromatography). The salt concentration, pH, pI, charge and/or ionic strength at peak elution of the AAV vector or capsid polypeptide can then be assessed so as to determine the binding capacity of the AAV vector or capsid polypeptide. In particular embodiments, the NaCl concentration and/or the conductivity at peak elution can then be determined.

In other examples, the binding capacity of AAV vector for heparin is assessed by surface plasmon resonance, e.g. with a BIAcore system, utilizing a solid support, such as a chip, to which heparin, heparan sulphate and/or HSPG is attached. In such examples, the heparin binding capacity is expressed as an equilibrium dissociation constant (K_{D}).

When assessing the heparin binding capacity of a test AAV vector to determine whether it elutes from a heparin affinity chromatography medium at the NaCl concentrations or conductivity described above, or to compare it to the heparin binding capacity of a reference AAV vector, the heparin binding capacity of the test vector need not be assessed using a binding assay that utilises a heparin affinity chromatography medium and elution with NaCl or another salt. It is understood that a different means for assessing the heparin binding capacity of a test AAV vector, e.g. surface plasmon resonance, can be used, provided the heparin binding results obtained with the different means have been correlated with or standardised to heparin binding results obtained using a binding assay that utilises a heparin affinity chromatography medium and elution with NaCl or another salt.

The methods can further include assessing the ability of the candidate AAV vector to transduce human hepatocytes in vivo. This can be done using small animal models, such as small animals (e.g. mouse, rat, guinea pig, rabbit, ferret, hamster, etc.) that have a chimeric liver that comprises human hepatocytes. Such models are known in the art and include, but are not limited to, albumin enhancer promoter-driven urokinase plasminogen activator transgenic mice (uPA-Tg mice) repopulated with human hepatocytes, Fah^{-/-}/Rag2^{-/-}/Il2rg^{-/-} (FRG) mice repopulated with human hepatocytes (hFRG mice), urokinase-type plasminogen activator/severe combined immunodeficiency (uPA/SCID) chimeric mice repopulated with human hepatocytes, hemizygous cDNA-uPA/SCID chimeric mice repopulated with human hepatocytes.

### 3. Use of Wild-Type AAV2 Vectors in Therapy

While there is substantial evidence that liver is a major target organ for AAV2, including the identification of liver-specific enhancer-promoter activity in the 3′ untranslated region of prototypic AAV2 (Logan et al. Nat Genet. 2017 August; 49(8):1267-1273), when tested in vivo on human hepatocytes using the FRG mouse xenografted liver model, AAV2 vectors containing the prototypic capsid exhibit very poor transduction (see Examples below). The low in vivo transduction of primary human hepatocytes is supported by what was observed in a clinical trial for haemophilia B in which prototypic AAV2 vector was used and led to low expression of therapeutic protein (Manno et al. Nat Med. 2006 March; 12(3):342-7). Thus, prototypic AAV2 has not been considered as a viable vector for delivery of heterologous coding sequences to the liver of human subjects, such as for gene therapy applications.

However, as determined herein for the first time, it is likely that the prototypic AAV2 having a capsid sequence set forth in SEQ ID NO:1 is actually a culture-adapted isolate and not a “true wild-type” AAV2. As demonstrated herein, AAV vectors comprising the prototypic AAV2 capsid have a high binding affinity for heparin. While this translates to effective transduction of human hepatocytes in vitro, transduction of hepatocytes in vivo is relatively poor, possibly due to sequestration of vectors by HSPG in vivo before effective transduction of target cells is achieved, as discussed above. Notably, modification of the prototypic AAV2 capsid polypeptide at one or more amino acid positions identified herein as being involved in heparin/HSPG binding can reduce the ability of a vector comprising the now-modified capsid polypeptide to bind to heparin/HSPG and increase the in vivo transduction efficiency of the vector (compared to a vector containing the prototypic AAV2 capsid polypeptide). Moreover, as demonstrated herein, novel AAV2 capsid polypeptides identified from primary human liver samples can be vectorised to produce AAV vectors that have reduced binding to heparin/HSPG compared to a vector containing the prototypic AAV2 capsid polypeptide. Importantly, these AAV vectors are very effective at transducing human hepatocytes in vivo, while being less effective at in vitro transduction.

Thus, without being bound by theory, it is postulated that the prototypic AAV2 having a capsid comprising a polypeptide set forth in SEQ ID NO:1 is culture-adapted, having accumulated amino acid substitutions that increase binding affinity for heparin/HPSG. While this promotes infection/transduction of hepatocytes in vitro, it impairs infection/transduction of hepatocytes in vivo. Conversely, non-culture-adapted wild-type AAV2 isolates, such as those isolated directly from primary human hepatocytes, typically are very effective at infecting/transducing human hepatocytes in vivo.

Consequently, it is determined herein for the first time that vectors produced by vectorising the capsid polypeptide from non-culture-adapted wild-type AAV2 and AAV2-like variants (such as using the methods for vectorising capsid polypeptides described above) are effective at delivering a heterologous coding sequence to a human hepatocyte in vivo, such as for gene therapy applications. This is in contrast to the previous understanding in the art that wild-type AAV2 vectors are not suitable for delivering a heterologous coding sequence to the human liver in vivo. Suitable wild-type AAV2 capsid polypeptides and AAV2-like variants can be isolated from human liver samples using well known-methods, such as those described in the Examples below.

Thus, provided herein is a method for delivering a heterologous coding sequence to a hepatocyte in a human subject, comprising administering to human subject an AAV vector comprising a heterologous coding sequence, wherein the AAV vector comprises a capsid comprising a wild-type AAV2 capsid polypeptide comprising an amino acid sequence having at least or about 90%, 91%, 92%, 93%, 94%, 95%, 96%, 97%, 98% or 99% sequence identity to the wild-type AAV2 capsid polypeptide set forth in SEQ ID NO:5. In an exemplary embodiment, the capsid polypeptide comprises an amino acid residue at the position corresponding to position 585 and/or position 588 of SEQ ID NO:5 that is not an arginine, i.e. is selected from among with a histidine (H), lysine (K), aspartic acid (D), glutamic acid (E), serine (S), threonine (T), asparagine (N), glutamine (Q), cysteine (C), selenocysteine (U), glycine (G), proline (P), alanine (A), valine (V), isoleucine (I), leucine (L), methionine (M), phenylalanine (F), tyrosine (Y) and tryptophan (W).

Typically, the delivery of the heterologous coding sequence to the hepatocyte is for therapeutic purposes, whereby expression of the heterologous coding sequence results in the treatment of a disease or condition in the subject. Thus, the AAV vectors can be administered to a human subject with a liver-associated disease or condition amendable to treatment with a protein, peptide or polynucleotide encoded by a heterologous coding sequence described herein.

In illustrative embodiments, the liver-associated disease or condition is selected from among a urea cycle disorder (UCD; including N-acetylglutamate synthase deficiency (NAGSD), carbamoylphosphate synthetase 1 deficiency (CPS1D), ornithine transcarbamylase deficiency (OTCD), argininosuccinate synthetase deficiency (ASSD), argininosuccinate lyase (ASLD), arginase 1 deficiency (ARG1D), citrin or aspartate/glutamate carrier deficiency and the mitochondrial ornithine transporter 1 deficiency causing hyperornithinemia-hyperammonemia-homocitrullinuria syndrome (HHH syndrome)), organic acidopathy (or organic academia, including methylmalonic acidemia, propionic acidemia, isovaleric acidemia, and maple syrup urine disease), aminoacidopathy, glycogenoses (Types I, III and IV), Wilson's disease, Progressive Familial Intrahepatic Cholestasis, primary hyperoxaluria, complementopathy, coagulopathy (e.g. hemophilia A, hemophilia B, von Willebrand disease (VWD)), Crigler Najjar syndrome, familial hypercholesterolaemia, α-1-antitrypsin deficiency, mitochondria respiratory chain hepatopathy, and citrin deficiency. Those skilled in the art would readily be able to select an appropriate heterologous coding sequence useful for treating such diseases. In some examples, the heterologous coding sequence comprises all or a part of a gene that is associated with the disease, such as all or a part of a gene set forth in Table 2. Introduction of such a sequence to the liver can be used for gene replacement or gene editing/correction, e.g. using CRISPR-Cas9. In particular examples, the heterologous coding sequence encodes a protein encoded by a gene that is associated with the disease, such as a gene set forth in Table 2.

TABLE 2

| Exemplary liver-associated | |
|---|---|
| diseases Indications | Exemplary associated genes |
| Urea cycle disorders (UCDs) | OTC, ASS, CPS1, ASL, ARG1 |
| Organic acidopathies | PCCA, PCCB, MMUT |
| Aminoacidopathies | PAH, FAH |
| Glycogenoses (Types I, III and IV) | SLC37A4 |
| Wilson's Disease | ATP7B |
| Progressive Familial Intrahepatic | ABCB4, ABCB11, ATP8B1 |
| Cholestasis | |
| Primary Hyperoxaluria | AGXT |
| Complementopathies | CFH, CFI |
| Coagulopathies | F8, F9, VWF |
| Crigler Najjar syndrome | UGT1A1 |
| Familial Hypercholesterolaemia | LDLR |
| a-1-antitrypsin Deficiency | SERPINA1 |
| Mitochondria Respiratory | POLG |
| Chain Hepatopathies | |
| Citrin Deficiency | SLC25A13 |

When used in vivo, titers of AAV vectors to be administered to a subject will vary depending on, for example, the particular recombinant virus, the disease or disorder to be treated, the mode of administration, the treatment goal, the individual to be treated, and the cell type(s) being targeted, and can be determined by methods well known to those skilled in the art. Although the exact dosage will be determined on an individual basis, in most cases, typically, recombinant viruses of the present disclosure can be administered to a subject at a dose of between 1×10¹⁰ genome copies of the recombinant virus per kg of the subject and 1×10¹⁴ genome copies per kg.

The route of the administration is not particularly limited. For example, a therapeutically effective amount of the AAV vector can be administered to the subject via, intramuscular, intravaginal, intravenous, intraperitoneal, subcutaneous, epicutaneous, intradermal, rectal, intraocular, pulmonary, intracranial, intraosseous, oral, buccal, or nasal routes. The AAV vector can be administrated as a single dose or multiple doses, and at varying intervals.

### 4. Capsid Polypeptides

The present disclosure is predicated in part on the identification of novel AAV capsid polypeptides. Typically, the capsid polypeptides, when present in the capsid of an AAV vector, facilitate efficient transduction of human cells (such as human hepatocytes). The in vivo transduction efficiency of AAV vectors having a capsid comprising a capsid polypeptide of the present disclosure is generally increased or enhanced compared to AAV vectors comprising a reference AAV capsid polypeptide (e.g. one set forth in SEQ ID NO:1, 55 or 56). The transduction efficiency can be enhanced by at least or about, 10%, 20%, 30%, 40%, 50%, 60%, 70%, 80%, 90%, 100%, 200%, 300%, 400%, 500%, 600%, 700%, 800%, 900% 1,000%, or more, e.g. the transduction efficiency of the AAV vector comprising a capsid polypeptide of the present disclosure can be at least or about 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more efficient at transducing cells in vivo compared to a reference AAV capsid polypeptide (e.g. one set forth in SEQ ID NO:1, 55 or 56).

The capsid polypeptides of the present disclosure are therefore particularly useful in preparing AAV vectors, and in particular AAV vectors for gene therapy uses.

The AAV vectors comprising a capsid polypeptide of the present disclosure typically have an in vivo transduction efficiency that is enhanced compared to a reference AAV vector having a capsid comprising the reference capsid polypeptide.

Thus, provided herein are polypeptides, including isolated polypeptides, comprising all or a portion of an AAV capsid polypeptide set forth in any one of SEQ ID Nos: 2-23, including all or a portion of the VP1 protein (comprising amino acid residues corresponding to those at positions 1-735 of SEQ ID NO:1), VP2 protein (comprising amino acid residues corresponding to those at positions 138-735 of SEQ ID NO:1) and/or the VP3 protein (comprising amino acid residues corresponding to those at positions 203-735 of SEQ ID NO:1), and variants thereof, including variants comprising at least or about 85%, 86%, 87%, 88%, 89%, 90%, 91%, 92%, 93%, 94%, 95%, 96%, 97%, 98%, or 99% sequence identity to the VP1, VP2 or VP3 proteins described herein.

Capsid polypeptides of the disclosure include those comprising all or a portion of the VP1 protein set forth in SEQ ID NO:2 (also referred to as CMRI_01) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:2 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:2 or a functional fragment thereof; and capsid polypeptides comprising all or a portion of the VP3 protein set forth as amino acids 203-735 of SEQ ID NO:2 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:2 or a functional fragment thereof.

Capsid polypeptides of the disclosure also include those comprising all or a portion of the VP1 protein set forth in SEQ ID NO:3 (also referred to as CMRI_02) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:3 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:3 or a functional fragment thereof; and capsid polypeptides comprising all or a portion of the VP3 protein set forth as amino acids 203-735 of SEQ ID NO:3 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:3 or a functional fragment thereof.

Exemplary capsid polypeptides of the disclosure also include those comprising all or a portion of the VP1 protein set forth in SEQ ID NO:4 (also referred to as CMRI_03) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-734 of SEQ ID NO:4 (also referred to as CMRI03) or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-734 of SEQ ID NO:4 or a functional fragment thereof; and capsid polypeptides comprising all or a portion of the VP3 protein set forth as amino acids 203-734 of SEQ ID NO:4 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-734 of SEQ ID NO:4 or a functional fragment thereof.

Also provided herein are capsid polypeptides comprising all or a portion of the VP1 protein set forth in SEQ ID NO:5 (also referred to as CMRI_04) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:5 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:5 or a functional fragment thereof; and capsid polypeptides comprising all or a portion of the VP3 protein set forth as amino acids 203-735 of SEQ ID NO:5 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:5 or a functional fragment thereof.

Capsid polypeptides of the disclosure also include those comprising all or a portion of the VP1 protein set forth in SEQ ID NO:6 (also referred to as CMRI_05) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:6 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:6 or a functional fragment thereof; and capsid polypeptides comprising all or a portion of the VP3 protein set forth as amino acids 203-735 of SEQ ID NO:6 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:6 or a functional fragment thereof.

Capsid polypeptides of the disclosure also include those comprising all or a portion of the VP1 protein set forth in SEQ ID NO:7 (also referred to as CMRI_06) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:7 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:7 or a functional fragment thereof; and capsid polypeptides comprising all or a portion of the VP3 protein set forth as amino acids 203-735 of SEQ ID NO:7 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:7 or a functional fragment thereof.

Other exemplary capsid polypeptides of the disclosure include those comprising all or a portion of the VP1 protein set forth in SEQ ID NO:8 (also referred to as CMRI_07) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:8 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:8 or a functional fragment thereof; and capsid polypeptides comprising all or a portion of the VP3 protein set forth as amino acids 203-735 of SEQ ID NO:8 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:8 or a functional fragment thereof.

Further exemplary capsid polypeptides of the disclosure include those comprising all or a portion of the VP1 protein set forth in SEQ ID NO:9 (also referred to as CMRI_08) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:9 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:9 or a functional fragment thereof; and capsid polypeptides comprising all or a portion of the VP3 protein set forth as amino acids 203-735 of SEQ ID NO:9 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:9 or a functional fragment thereof.

Capsid polypeptides of the present disclosure also include those comprising all or a portion of the VP1 protein set forth in SEQ ID NO:10 (also referred to as CMRI_09) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:10 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:10 or a functional fragment thereof; and capsid polypeptides comprising all or a portion of the VP3 protein set forth as amino acids 203-735 of SEQ ID NO:10 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:10 or a functional fragment thereof.

Capsid polypeptides of the present disclosure also include those comprising all or a portion of the VP1 protein set forth in SEQ ID NO:11 (also referred to as CMRI_10) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:11 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:11 or a functional fragment thereof; and capsid polypeptides comprising all or a portion of the VP3 protein set forth as amino acids 203-735 of SEQ ID NO:11 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:11 or a functional fragment thereof.

Exemplary capsid polypeptides of the present disclosure also include those comprising all or a portion of the VP1 protein set forth in SEQ ID NO:12 (also referred to as CMRI_11) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-737 of SEQ ID NO:12 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-737 of SEQ ID NO:12 or a functional fragment thereof; and capsid polypeptides comprising all or a portion of the VP3 protein set forth as amino acids 204-737 of SEQ ID NO:12 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-737 of SEQ ID NO:12 or a functional fragment thereof.

Further exemplary capsid polypeptides of the present disclosure include those comprising all or a portion of the VP1 protein set forth in SEQ ID NO:13 (also referred to as CMRI_12) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-737 of SEQ ID NO:13 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-737 of SEQ ID NO:13 or a functional fragment thereof; and capsid polypeptides comprising all or a portion of the VP3 protein set forth as amino acids 204-737 of SEQ ID NO:13 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-737 of SEQ ID NO:13 or a functional fragment thereof.

Also provided are capsid polypeptides that comprise all or a portion of the VP1 protein set forth in SEQ ID NO:14 (also referred to as CMRI_13) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-737 of SEQ ID NO:14 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-737 of SEQ ID NO:14 or a functional fragment thereof; and capsid polypeptides comprising all or a portion of the VP3 protein set forth as amino acids 204-737 of SEQ ID NO:14 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-737 of SEQ ID NO:14 or a functional fragment thereof.

Capsid polypeptides of the present disclosure also include those that comprise all or a portion of the VP1 protein set forth in SEQ ID NO:15 (also referred to as CMRI_14) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-737 of SEQ ID NO:15 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-737 of SEQ ID NO:15 or a functional fragment thereof; and capsid polypeptides comprising all or a portion of the VP3 protein set forth as amino acids 204-737 of SEQ ID NO:15 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-737 of SEQ ID NO:15 or a functional fragment thereof.

Capsid polypeptides of the present disclosure also include those that comprise all or a portion of the VP1 protein set forth in SEQ ID NO:16 (also referred to as CMRI_15) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-736 of SEQ ID NO:16 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-736 of SEQ ID NO:16 or a functional fragment thereof; and capsid polypeptides comprising all or a portion of the VP3 protein set forth as amino acids 204-736 of SEQ ID NO:16 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-736 of SEQ ID NO:16 or a functional fragment thereof.

Exemplary capsid polypeptides of the present disclosure also include those that comprise all or a portion of the VP1 protein set forth in SEQ ID NO:17 (also referred to as CMRI_16) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-737 of SEQ ID NO:17 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-737 of SEQ ID NO:17 or a functional fragment thereof; and capsid polypeptides comprising all or a portion of the VP3 protein set forth as amino acids 204-737 of SEQ ID NO:17 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-737 of SEQ ID NO:17 or a functional fragment thereof.

Exemplary capsid polypeptides also include those comprising all or a portion of the VP1 protein set forth in SEQ ID NO:18 (also referred to as CMRI_17) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-737 of SEQ ID NO:18 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-737 of SEQ ID NO:18 or a functional fragment thereof; and capsid polypeptides comprising all or a portion of the VP3 protein set forth as amino acids 204-737 of SEQ ID NO:18 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-737 of SEQ ID NO:18 or a functional fragment thereof.

Further exemplary capsid polypeptides of the present disclosure include those comprising all or a portion of the VP1 protein set forth in SEQ ID NO:19 (also referred to as CMRI_18) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:19 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:19 or a functional fragment thereof; and capsid polypeptides comprising all or a portion of the VP3 protein set forth as amino acids 203-735 of SEQ ID NO:19 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:19 or a functional fragment thereof.

Capsid polypeptides of the present disclosure also include those comprising all or a portion of the VP1 protein set forth in SEQ ID NO:20 (also referred to as CMRI_19) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:20 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:20 or a functional fragment thereof; and capsid polypeptides comprising all or a portion of the VP3 protein set forth as amino acids 203-735 of SEQ ID NO:20 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:20 or a functional fragment thereof.

Also provided are capsid polypeptides comprising all or a portion of the VP1 protein set forth in SEQ ID NO:21 (also referred to as CMRI_20) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:21 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:21 or a functional fragment thereof; and capsid polypeptides comprising all or a portion of the VP3 protein set forth as amino acids 203-735 of SEQ ID NO:21 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:21 or a functional fragment thereof.

Also provided are capsid polypeptides comprising all or a portion of the VP1 protein set forth in SEQ ID NO:22 (also referred to as CMRI_21) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:22 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:22 or a functional fragment thereof; and capsid polypeptides comprising all or a portion of the VP3 protein set forth as amino acids 203-735 of SEQ ID NO:22 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:22 or a functional fragment thereof.

Also provided are capsid polypeptides comprising all or a portion of the VP1 protein set forth in SEQ ID NO:23 (also referred to as CMRI_22) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:23 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:23 or a functional fragment thereof; and capsid polypeptides comprising all or a portion of the VP3 protein set forth as amino acids 203-735 of SEQ ID NO:23 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:23 or a functional fragment thereof.

Also provided are capsid polypeptides comprising all or a portion of the VP1 protein set forth in SEQ ID NO:24 (also referred to as CMRI_23) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:24 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:24 or a functional fragment thereof; and capsid polypeptides comprising all or a portion of the VP3 protein set forth as amino acids 203-735 of SEQ ID NO:24 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:24 or a functional fragment thereof.

Provided are capsid polypeptides comprising all or a portion of the VP1 protein set forth in SEQ ID NO:25 (also referred to as CMRI_24) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:25 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:25 or a functional fragment thereof; and capsid polypeptides comprising all or a portion of the VP3 protein set forth as amino acids 203-735 of SEQ ID NO:25 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:25 or a functional fragment thereof.

Capsid polypeptides of the present disclosure also include those comprising all or a portion of the VP1 protein set forth in SEQ ID NO:26 (also referred to as CMRI_25) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:26 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:26 or a functional fragment thereof; and capsid polypeptides comprising all or a portion of the VP3 protein set forth as amino acids 203-735 of SEQ ID NO:26 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:26 or a functional fragment thereof.

Also provided are capsid polypeptides comprising all or a portion of the VP1 protein set forth in SEQ ID NO:27 (also referred to as CMRI_26) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:27 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:27 or a functional fragment thereof; and capsid polypeptides comprising all or a portion of the VP3 protein set forth as amino acids 203-735 of SEQ ID NO:27 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:27 or a functional fragment thereof.

Also provided are capsid polypeptides comprising all or a portion of the VP1 protein set forth in SEQ ID NO:62 (also referred to as CMRI_27) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:62 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:62 or a functional fragment thereof; and capsid polypeptides comprising all or a portion of the VP3 protein set forth as amino acids 203-735 of SEQ ID NO:62 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:62 or a functional fragment thereof.

Also provided are capsid polypeptides comprising all or a portion of the VP1 protein set forth in SEQ ID NO:63 (also referred to as CMRI_28) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:63 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:63 or a functional fragment thereof; and capsid polypeptides comprising all or a portion of the VP3 protein set forth as amino acids 203-735 of SEQ ID NO:63 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:63 or a functional fragment thereof.

Additionally, provided are capsid polypeptides comprising all or a portion of the VP1 protein set forth in SEQ ID NO:64 (also referred to as CMRI_29) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:64 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:64 or a functional fragment thereof; and capsid polypeptides comprising all or a portion of the VP3 protein set forth as amino acids 203-735 of SEQ ID NO:64 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:64 or a functional fragment thereof.

Also provided are capsid polypeptides comprising all or a portion of the VP1 protein set forth in SEQ ID NO:65 (also referred to as CMRI_30) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:65 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:65 or a functional fragment thereof; and capsid polypeptides comprising all or a portion of the VP3 protein set forth as amino acids 203-735 of SEQ ID NO:65 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-735 of SEQ ID NO:65 or a functional fragment thereof.

Capsid polypeptides of the present disclosure also include those that comprise all or a portion of the VP1 protein set forth in SEQ ID NO:71 (also referred to as CMRI_31) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-736 of SEQ ID NO:71 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-736 of SEQ ID NO:71 or a functional fragment thereof; and capsid polypeptides comprising all or a portion of the VP3 protein set forth as amino acids 204-736 of SEQ ID NO:71 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-736 of SEQ ID NO:71 or a functional fragment thereof.

Capsid polypeptides of the present disclosure also include those that comprise all or a portion of the VP1 protein set forth in SEQ ID NO:72 (also referred to as CMRI_32) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-736 of SEQ ID NO:72 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-736 of SEQ ID NO:72 or a functional fragment thereof; and capsid polypeptides comprising all or a portion of the VP3 protein set forth as amino acids 204-736 of SEQ ID NO:72 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-736 of SEQ ID NO:72 or a functional fragment thereof.

Capsid polypeptides of the present disclosure also include those that comprise all or a portion of the VP1 protein set forth in SEQ ID NO:73 (also referred to as CMRI_33) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-736 of SEQ ID NO:73 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-736 of SEQ ID NO:73 or a functional fragment thereof; and capsid polypeptides comprising all or a portion of the VP3 protein set forth as amino acids 204-736 of SEQ ID NO:73 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-736 of SEQ ID NO:73 or a functional fragment thereof.

Capsid polypeptides of the present disclosure also include those that comprise all or a portion of the VP1 protein set forth in SEQ ID NO:74 (also referred to as CMRI_34) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-736 of SEQ ID NO:74 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-736 of SEQ ID NO:74 or a functional fragment thereof; and capsid polypeptides comprising all or a portion of the VP3 protein set forth as amino acids 204-736 of SEQ ID NO:74 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-736 of SEQ ID NO:74 or a functional fragment thereof.

Capsid polypeptides of the present disclosure also include those comprising 1, 2, 3, 4, 5, 6, 7, 8, 9, 10 or 11 modifications relative to the reference or prototypic AAV2 capsid polypeptide set forth in SEQ ID NO:1 (also Genbank Accession NC_001401), wherein the modifications are at positions selected from among those corresponding to positions 482, 484, 487, 496, 503, 532, 582, 585, 588, 589 and 596 of the prototypic AAV2 capsid polypeptide set forth in SEQ ID NO:1. The amino acid residue at any one or more of these positions may be substituted with any another amino acid residue. In particular examples, the capsid polypeptides comprise modifications at positions 482 and 496; 482 and 532; 482, 496 and 532; 482 and 585; 482, 503 and 596; 496, 588 and 596; 496, 532, 585, 588 and 596; or 503, 585, 588 and 596, relative to the prototypic AAV2 capsid polypeptide set forth in SEQ ID NO:1. As discussed above, an AAV vector comprising such a capsid polypeptide typically exhibits improved in vivo transduction efficiency compared to an AAV vector comprising a the prototypic AAV2 capsid polypeptide.

Exemplary modifications that may be present in a capsid polypeptide of the present disclosure therefore include those resulting in a serine (S) or threonine (T) at the position corresponding to position 482 of SEQ ID NO:1 (e.g. C482S or C482T relative to SEQ ID NO:1); those resulting in a glutamine (Q) or asparagine (N) at position 484 (e.g. R484Q or R484N relative to SEQ ID NO:1); those resulting in a glutamine (Q) or asparagine (N) at position 487 (e.g. R487Q or R487N relative to SEQ ID NO:1); those resulting in an aspartic acid (D) or glutamic acid (E) at the position corresponding to position 496 of SEQ ID NO:1 (e.g. N496D or N496E relative to SEQ ID NO:1); those resulting in an alanine (A), valine (V) or isoleucine (I) at the position corresponding to position 503 of SEQ ID NO:1 (e.g. T503A, T503V, or T503I relative to SEQ ID NO:1); those resulting in a glutamic acid (E) or glutamine (Q) at the position corresponding to position 532 of SEQ ID NO:1 (e.g. K532E or K532Q relative to SEQ ID NO:1); those resulting in a serine (S), aspartic acid (D), tyrosine (Y), or threonine (T) at the position corresponding to position 582 of SEQ ID NO:1 (e.g. N582S, N582D, N582Y or N582T relative to SEQ ID NO:1); those resulting in a serine (S), threonine (T), glycine (G), alanine (A) or glutamic acid (E) at the position corresponding to position 585 of SEQ ID NO:1 (e.g. R585S, R585T, R585G, R585A or R585E, relative to SEQ ID NO:1); those resulting in a threonine (T), isoleucine (I) or alanine (A) at the position corresponding to position 588 of SEQ ID NO:1 (e.g. R588T, R588I or R588A relative to SEQ ID NO:1); and those resulting in an aspartic acid (D), glutamic acid (E) or alanine (A) at the position corresponding to position 589 of SEQ ID NO:1 (e.g. Q589D, Q589E or Q589A relative to SEQ ID NO:1); and those resulting in an aspartic acid (D) or glutamic acid (E) at the position corresponding to position 596 of SEQ ID NO:1 (e.g. N596D or N596E relative to SEQ ID NO:1).

The capsid polypeptides may comprise 1, 2, 3, 4, 5, 6, 7, 8, 9, 10 or 11 of these modifications (i.e. modifications at positions selected from among those corresponding to positions 482, 484, 487, 496, 503, 532, 582, 585, 588, 589 and 596 of the prototypic AAV2 capsid polypeptide set forth in SEQ ID NO:1) in an AAV2 capsid polypeptide set forth in SEQ ID NO:1 or in a polypeptide having at least or about 85%, 86%, 87%, 88%, 89%, 90%, 91%, 92%, 93%, 94%, 95%, 96%, 97%, 98%, or 99% sequence identity to the sequence set forth in SEQ ID NO:1. Capsid polypeptides comprising such modifications include those comprising all or a portion of the VP1 protein (comprising amino acid residues corresponding to those at positions 1-735 of SEQ ID NO:1), VP2 protein (comprising amino acid residues corresponding to those at positions 138-735 of SEQ ID NO:1) and/or the VP3 protein (comprising amino acid residues corresponding to those at positions 203-735 of SEQ ID NO:1), including functional fragments thereof.

In particular embodiments of the present disclosure, the capsid polypeptides include those comprising 1, 2, 3, 4, 5, 7 or 8 amino acid modifications relative to the prototypic AAV2 capsid polypeptide set forth in SEQ ID NO:1 (also Genbank Accession NC_001401), wherein the modifications are at positions selected from among those corresponding to positions 482, 496, 503, 532, 582, 585, 588 and 596 of the prototypic AAV2 capsid polypeptide set forth in SEQ ID NO:1. In particular embodiments, the modifications are amino acid substitutions. Exemplary substitutions include those resulting in a serine (S) at the position corresponding to position 482 of SEQ ID NO:1 (e.g. C482S relative to SEQ ID NO:1); those resulting in an aspartic acid (D) at the position corresponding to position 496 of SEQ ID NO:1 (e.g. N496D relative to SEQ ID NO:1); those resulting in an alanine (A) at the position corresponding to position 503 of SEQ ID NO:1 (e.g. T503A relative to SEQ ID NO:1); those resulting in a glutamic acid (E) or glutamine (Q) at the position corresponding to position 532 of SEQ ID NO:1 (e.g. K532E or K532Q relative to SEQ ID NO:1); those resulting in a serine (S) at the position corresponding to position 582 of SEQ ID NO:1 (e.g. N582S relative to SEQ ID NO:1); those resulting in a serine (S) at the position corresponding to position 585 of SEQ ID NO:1 (e.g. R585S relative to SEQ ID NO:1); those resulting in a threonine (T) at the position corresponding to position 588 of SEQ ID NO:1 (e.g. R588T relative to SEQ ID NO:1); and those resulting in an aspartic acid (D) at the position corresponding to position 596 of SEQ ID NO:1 (e.g. N596D relative to SEQ ID NO:1). Exemplary capsid polypeptides of the present disclosure therefore include those that comprise the amino acids residues at positions corresponding to positions 482, 496, 503, 532, 582, 585, 588 and 596 of SEQ ID NO:1 as shown in Table 3.

TABLE 3
Amino acid residues at positions 482, 496, 503, 532, 582, 585, 588 and 596

| | Amino acid position (relative to SEQ ID NO: 1) | | | | | | | |
|---|---|---|---|---|---|---|---|---|
| | 482 | 496 | 503 | 532 | 582 | 585 | 588 | 596 |
| 1 | C | N | T | K | N | R | R | N |
| (prototypic) | | | | | | | | |
| 2 | C | N | T | K | N | R | R | D |
| 3 | C | N | T | K | N | R | T | N |
| 4 | C | N | T | K | N | R | T | D |
| 5 | C | N | T | K | N | S | R | N |
| 6 | C | N | T | K | N | S | R | D |
| 7 | C | N | T | K | N | S | T | N |
| 8 | C | N | T | K | N | S | T | D |
| 9 | C | N | T | K | S | R | R | N |
| 10 | C | N | T | K | S | R | R | D |
| 11 | C | N | T | K | S | R | T | N |
| 12 | C | N | T | K | S | R | T | D |
| 13 | C | N | T | K | S | S | R | N |
| 14 | C | N | T | K | S | S | R | D |
| 15 | C | N | T | K | S | S | T | N |
| 16 | C | N | T | K | S | S | T | D |
| 17 | C | N | T | E | N | R | R | N |
| 18 | C | N | T | E | N | R | R | D |
| 19 | C | N | T | E | N | R | T | N |
| 20 | C | N | T | E | N | R | T | D |
| 21 | C | N | T | E | N | S | R | N |
| 22 | C | N | T | E | N | S | R | D |
| 23 | C | N | T | E | N | S | T | N |
| 24 | C | N | T | E | N | S | T | D |
| 25 | C | N | T | E | S | R | R | N |
| 26 | C | N | T | E | S | R | R | D |
| 27 | C | N | T | E | S | R | T | N |
| 28 | C | N | T | E | S | R | T | D |
| 29 | C | N | T | E | S | S | R | N |
| 30 | C | N | T | E | S | S | R | D |
| 31 | C | N | T | E | S | S | T | N |
| 32 | C | N | T | E | S | S | T | D |
| 33 | C | N | T | Q | N | R | R | N |
| 34 | C | N | T | Q | N | R | R | D |
| 35 | C | N | T | Q | N | R | T | N |
| 36 | C | N | T | Q | N | R | T | D |
| 37 | C | N | T | Q | N | S | R | N |
| 38 | C | N | T | Q | N | S | R | D |
| 39 | C | N | T | Q | N | S | T | N |
| 40 | C | N | T | Q | N | S | T | D |
| 41 | C | N | T | Q | S | R | R | N |
| 42 | C | N | T | Q | S | R | R | D |
| 43 | C | N | T | Q | S | R | T | N |
| 44 | C | N | T | Q | S | R | T | D |
| 45 | C | N | T | Q | S | S | R | N |
| 46 | C | N | T | Q | S | S | R | D |
| 47 | C | N | T | Q | S | S | T | N |
| 48 | C | N | T | Q | S | S | T | D |
| 49 | C | N | A | K | N | R | R | N |
| 50 | C | N | A | K | N | R | R | D |
| 51 | C | N | A | K | N | R | T | N |
| 52 | C | N | A | K | N | R | T | D |
| 53 | C | N | A | K | N | S | R | N |
| 54 | C | N | A | K | N | S | R | D |
| 55 | C | N | A | K | N | S | T | N |
| 56 | C | N | A | K | N | S | T | D |
| 57 | C | N | A | K | S | R | R | N |
| 58 | C | N | A | K | S | R | R | D |
| 59 | C | N | A | K | S | R | T | N |
| 60 | C | N | A | K | S | R | T | D |
| 61 | C | N | A | K | S | S | R | N |
| 62 | C | N | A | K | S | S | R | D |
| 63 | C | N | A | K | S | S | T | N |
| 64 | C | N | A | K | S | S | T | D |
| 65 | C | N | A | E | N | R | R | N |
| 66 | C | N | A | E | N | R | R | D |
| 67 | C | N | A | E | N | R | T | N |
| 68 | C | N | A | E | N | R | T | D |
| 69 | C | N | A | E | N | S | R | N |
| 70 | C | N | A | E | N | S | R | D |
| 71 | C | N | A | E | N | S | T | N |
| 72 | C | N | A | E | N | S | T | D |
| 73 | C | N | A | E | S | R | R | N |
| 74 | C | N | A | E | S | R | R | D |
| 75 | C | N | A | E | S | R | T | N |
| 76 | C | N | A | E | S | R | T | D |
| 77 | C | N | A | E | S | S | R | N |
| 78 | C | N | A | E | S | S | R | D |
| 79 | C | N | A | E | S | S | T | N |
| 80 | C | N | A | E | S | S | T | D |
| 81 | C | N | A | Q | N | R | R | N |
| 82 | C | N | A | Q | N | R | R | D |
| 83 | C | N | A | Q | N | R | T | N |
| 84 | C | N | A | Q | N | R | T | D |
| 85 | C | N | A | Q | N | S | R | N |
| 86 | C | N | A | Q | N | S | R | D |
| 87 | C | N | A | Q | N | S | T | N |
| 88 | C | N | A | Q | N | S | T | D |
| 89 | C | N | A | Q | S | R | R | N |
| 90 | C | N | A | Q | S | R | R | D |
| 91 | C | N | A | Q | S | R | T | N |
| 92 | C | N | A | Q | S | R | T | D |
| 93 | C | N | A | Q | S | S | R | N |
| 94 | C | N | A | Q | S | S | R | D |
| 95 | C | N | A | Q | S | S | T | N |
| 96 | C | N | A | Q | S | S | T | D |
| 97 | C | D | T | K | N | R | R | N |
| 98 | C | D | T | K | N | R | R | D |
| 99 | C | D | T | K | N | R | T | N |
| 100 | C | D | T | K | N | R | T | D |
| 101 | C | D | T | K | N | S | R | N |
| 102 | C | D | T | K | N | S | R | D |
| 103 | C | D | T | K | N | S | T | N |
| 104 | C | D | T | K | N | S | T | D |
| 105 | C | D | T | K | S | R | R | N |
| 106 | C | D | T | K | S | R | R | D |
| 107 | C | D | T | K | S | R | T | N |
| 108 | C | D | T | K | S | R | T | D |
| 109 | C | D | T | K | S | S | R | N |
| 110 | C | D | T | K | S | S | R | D |
| 111 | C | D | T | K | S | S | T | N |
| 112 | C | D | T | K | S | S | T | D |
| 113 | C | D | T | E | N | R | R | N |
| 114 | C | D | T | E | N | R | R | D |
| 115 | C | D | T | E | N | R | T | N |
| 116 | C | D | T | E | N | R | T | D |
| 117 | C | D | T | E | N | S | R | N |
| 118 | C | D | T | E | N | S | R | D |
| 119 | C | D | T | E | N | S | T | N |
| 120 | C | D | T | E | N | S | T | D |
| 121 | C | D | T | E | S | R | R | N |
| 122 | C | D | T | E | S | R | R | D |
| 123 | C | D | T | E | S | R | T | N |
| 124 | C | D | T | E | S | R | T | D |
| 125 | C | D | T | E | S | S | R | N |
| 126 | C | D | T | E | S | S | R | D |
| 127 | C | D | T | E | S | S | T | N |
| 128 | C | D | T | E | S | S | T | D |
| 129 | C | D | T | Q | N | R | R | N |
| 130 | C | D | T | Q | N | R | R | D |
| 131 | C | D | T | Q | N | R | T | N |
| 132 | C | D | T | Q | N | R | T | D |
| 133 | C | D | T | Q | N | S | R | N |
| 134 | C | D | T | Q | N | S | R | D |
| 135 | C | D | T | Q | N | S | T | N |
| 136 | C | D | T | Q | N | S | T | D |
| 137 | C | D | T | Q | S | R | R | N |
| 138 | C | D | T | Q | S | R | R | D |
| 139 | C | D | T | Q | S | R | T | N |
| 140 | C | D | T | Q | S | R | T | D |
| 141 | C | D | T | Q | S | S | R | N |
| 142 | C | D | T | Q | S | S | R | D |
| 143 | C | D | T | Q | S | S | T | N |
| 144 | C | D | T | Q | S | S | T | D |
| 145 | C | D | A | K | N | R | R | N |
| 146 | C | D | A | K | N | R | R | D |
| 147 | C | D | A | K | N | R | T | N |
| 148 | C | D | A | K | N | R | T | D |
| 149 | C | D | A | K | N | S | R | N |
| 150 | C | D | A | K | N | S | R | D |
| 151 | C | D | A | K | N | S | T | N |
| 152 | C | D | A | K | N | S | T | D |
| 153 | C | D | A | K | S | R | R | N |
| 154 | C | D | A | K | S | R | R | D |
| 155 | C | D | A | K | S | R | T | N |
| 156 | C | D | A | K | S | R | T | D |
| 157 | C | D | A | K | S | S | R | N |
| 158 | C | D | A | K | S | S | R | D |
| 159 | C | D | A | K | S | S | T | N |
| 160 | C | D | A | K | S | S | T | D |
| 161 | C | D | A | E | N | R | R | N |
| 162 | C | D | A | E | N | R | R | D |
| 163 | C | D | A | E | N | R | T | N |
| 164 | C | D | A | E | N | R | T | D |
| 165 | C | D | A | E | N | S | R | N |
| 166 | C | D | A | E | N | S | R | D |
| 167 | C | D | A | E | N | S | T | N |
| 168 | C | D | A | E | N | S | T | D |
| 169 | C | D | A | E | S | R | R | N |
| 170 | C | D | A | E | S | R | R | D |
| 171 | C | D | A | E | S | R | T | N |
| 172 | C | D | A | E | S | R | T | D |
| 173 | C | D | A | E | S | S | R | N |
| 174 | C | D | A | E | S | S | R | D |
| 175 | C | D | A | E | S | S | T | N |
| 176 | C | D | A | E | S | S | T | D |
| 177 | C | D | A | Q | N | R | R | N |
| 178 | C | D | A | Q | N | R | R | D |
| 179 | C | D | A | Q | N | R | T | N |
| 180 | C | D | A | Q | N | R | T | D |
| 181 | C | D | A | Q | N | S | R | N |
| 182 | C | D | A | Q | N | S | R | D |
| 183 | C | D | A | Q | N | S | T | N |
| 184 | C | D | A | Q | N | S | T | D |
| 185 | C | D | A | Q | S | R | R | N |
| 186 | C | D | A | Q | S | R | R | D |
| 187 | C | D | A | Q | S | R | T | N |
| 188 | C | D | A | Q | S | R | T | D |
| 189 | C | D | A | Q | S | S | R | N |
| 190 | C | D | A | Q | S | S | R | D |
| 191 | C | D | A | Q | S | S | T | N |
| 192 | C | D | A | Q | S | S | T | D |
| 193 | S | N | T | K | N | R | R | N |
| 194 | S | N | T | K | N | R | R | D |
| 195 | S | N | T | K | N | R | T | N |
| 196 | S | N | T | K | N | R | T | D |
| 197 | S | N | T | K | N | S | R | N |
| 198 | S | N | T | K | N | S | R | D |
| 199 | S | N | T | K | N | S | T | N |
| 200 | S | N | T | K | N | S | T | D |
| 201 | S | N | T | K | S | R | R | N |
| 202 | S | N | T | K | S | R | R | D |
| 203 | S | N | T | K | S | R | T | N |
| 204 | S | N | T | K | S | R | T | D |
| 205 | S | N | T | K | S | S | R | N |
| 206 | S | N | T | K | S | S | R | D |
| 207 | S | N | T | K | S | S | T | N |
| 208 | S | N | T | K | S | S | T | D |
| 209 | S | N | T | E | N | R | R | N |
| 210 | S | N | T | E | N | R | R | D |
| 211 | S | N | T | E | N | R | T | N |
| 212 | S | N | T | E | N | R | T | D |
| 213 | S | N | T | E | N | S | R | N |
| 214 | S | N | T | E | N | S | R | D |
| 215 | S | N | T | E | N | S | T | N |
| 216 | S | N | T | E | N | S | T | D |
| 217 | S | N | T | E | S | R | R | N |
| 218 | S | N | T | E | S | R | R | D |
| 219 | S | N | T | E | S | R | T | N |
| 220 | S | N | T | E | S | R | T | D |
| 221 | S | N | T | E | S | S | R | N |
| 222 | S | N | T | E | S | S | R | D |
| 223 | S | N | T | E | S | S | T | N |
| 224 | S | N | T | E | S | S | T | D |
| 225 | S | N | T | Q | N | R | R | N |
| 226 | S | N | T | Q | N | R | R | D |
| 227 | S | N | T | Q | N | R | T | N |
| 228 | S | N | T | Q | N | R | T | D |
| 229 | S | N | T | Q | N | S | R | N |
| 230 | S | N | T | Q | N | S | R | D |
| 231 | S | N | T | Q | N | S | T | N |
| 232 | S | N | T | Q | N | S | T | D |
| 233 | S | N | T | Q | S | R | R | N |
| 234 | S | N | T | Q | S | R | R | D |
| 235 | S | N | T | Q | S | R | T | N |
| 236 | S | N | T | Q | S | R | T | D |
| 237 | S | N | T | Q | S | S | R | N |
| 238 | S | N | T | Q | S | S | R | D |
| 239 | S | N | T | Q | S | S | T | N |
| 240 | S | N | T | Q | S | S | T | D |
| 241 | S | N | A | K | N | R | R | N |
| 242 | S | N | A | K | N | R | R | D |
| 243 | S | N | A | K | N | R | T | N |
| 244 | S | N | A | K | N | R | T | D |
| 245 | S | N | A | K | N | S | R | N |
| 246 | S | N | A | K | N | S | R | D |
| 247 | S | N | A | K | N | S | T | N |
| 248 | S | N | A | K | N | S | T | D |
| 249 | S | N | A | K | S | R | R | N |
| 250 | S | N | A | K | S | R | R | D |
| 251 | S | N | A | K | S | R | T | N |
| 252 | S | N | A | K | S | R | T | D |
| 253 | S | N | A | K | S | S | R | N |
| 254 | S | N | A | K | S | S | R | D |
| 255 | S | N | A | K | S | S | T | N |
| 256 | S | N | A | K | S | S | T | D |
| 257 | S | N | A | E | N | R | R | N |
| 258 | S | N | A | E | N | R | R | D |
| 259 | S | N | A | E | N | R | T | N |
| 260 | S | N | A | E | N | R | T | D |
| 261 | S | N | A | E | N | S | R | N |
| 262 | S | N | A | E | N | S | R | D |
| 263 | S | N | A | E | N | S | T | N |
| 264 | S | N | A | E | N | S | T | D |
| 265 | S | N | A | E | S | R | R | N |
| 266 | S | N | A | E | S | R | R | D |
| 267 | S | N | A | E | S | R | T | N |
| 268 | S | N | A | E | S | R | T | D |
| 269 | S | N | A | E | S | S | R | N |
| 270 | S | N | A | E | S | S | R | D |
| 271 | S | N | A | E | S | S | T | N |
| 272 | S | N | A | E | S | S | T | D |
| 273 | S | N | A | Q | N | R | R | N |
| 274 | S | N | A | Q | N | R | R | D |
| 275 | S | N | A | Q | N | R | T | N |
| 276 | S | N | A | Q | N | R | T | D |
| 277 | S | N | A | Q | N | S | R | N |
| 278 | S | N | A | Q | N | S | R | D |
| 279 | S | N | A | Q | N | S | T | N |
| 280 | S | N | A | Q | N | S | T | D |
| 281 | S | N | A | Q | S | R | R | N |
| 282 | S | N | A | Q | S | R | R | D |
| 283 | S | N | A | Q | S | R | T | N |
| 284 | S | N | A | Q | S | R | T | D |
| 285 | S | N | A | Q | S | S | R | N |
| 286 | S | N | A | Q | S | S | R | D |
| 287 | S | N | A | Q | S | S | T | N |
| 288 | S | N | A | Q | S | S | T | D |
| 289 | S | D | T | K | N | R | R | N |
| 290 | S | D | T | K | N | R | R | D |
| 291 | S | D | T | K | N | R | T | N |
| 292 | S | D | T | K | N | R | T | D |
| 293 | S | D | T | K | N | S | R | N |
| 294 | S | D | T | K | N | S | R | D |
| 295 | S | D | T | K | N | S | T | N |
| 296 | S | D | T | K | N | S | T | D |
| 297 | S | D | T | K | S | R | R | N |
| 298 | S | D | T | K | S | R | R | D |
| 299 | S | D | T | K | S | R | T | N |
| 300 | S | D | T | K | S | R | T | D |
| 301 | S | D | T | K | S | S | R | N |
| 302 | S | D | T | K | S | S | R | D |
| 303 | S | D | T | K | S | S | T | N |
| 304 | S | D | T | K | S | S | T | D |
| 305 | S | D | T | E | N | R | R | N |
| 306 | S | D | T | E | N | R | R | D |
| 307 | S | D | T | E | N | R | T | N |
| 308 | S | D | T | E | N | R | T | D |
| 309 | S | D | T | E | N | S | R | N |
| 310 | S | D | T | E | N | S | R | D |
| 311 | S | D | T | E | N | S | T | N |
| 312 | S | D | T | E | N | S | T | D |
| 313 | S | D | T | E | S | R | R | N |
| 314 | S | D | T | E | S | R | R | D |
| 315 | S | D | T | E | S | R | T | N |
| 316 | S | D | T | E | S | R | T | D |
| 317 | S | D | T | E | S | S | R | N |
| 318 | S | D | T | E | S | S | R | D |
| 319 | S | D | T | E | S | S | T | N |
| 320 | S | D | T | E | S | S | T | D |
| 321 | S | D | T | Q | N | R | R | N |
| 322 | S | D | T | Q | N | R | R | D |
| 323 | S | D | T | Q | N | R | T | N |
| 324 | S | D | T | Q | N | R | T | D |
| 325 | S | D | T | Q | N | S | R | N |
| 326 | S | D | T | Q | N | S | R | D |
| 327 | S | D | T | Q | N | S | T | N |
| 328 | S | D | T | Q | N | S | T | D |
| 329 | S | D | T | Q | S | R | R | N |
| 330 | S | D | T | Q | S | R | R | D |
| 331 | S | D | T | Q | S | R | T | N |
| 332 | S | D | T | Q | S | R | T | D |
| 333 | S | D | T | Q | S | S | R | N |
| 334 | S | D | T | Q | S | S | R | D |
| 335 | S | D | T | Q | S | S | T | N |
| 336 | S | D | T | Q | S | S | T | D |
| 337 | S | D | A | K | N | R | R | N |
| 338 | S | D | A | K | N | R | R | D |
| 339 | S | D | A | K | N | R | T | N |
| 340 | S | D | A | K | N | R | T | D |
| 341 | S | D | A | K | N | s | R | N |
| 342 | S | D | A | K | N | S | R | D |
| 343 | S | D | A | K | N | S | T | N |
| 344 | S | D | A | K | N | S | T | D |
| 345 | S | D | A | K | S | R | R | N |
| 346 | S | D | A | K | S | R | R | D |
| 347 | S | D | A | K | S | R | T | N |
| 348 | S | D | A | K | S | R | T | D |
| 349 | S | D | A | K | S | S | R | N |
| 350 | S | D | A | K | S | S | R | D |
| 351 | S | D | A | K | S | S | T | N |
| 352 | S | D | A | K | S | S | T | D |
| 353 | S | D | A | E | N | R | R | N |
| 354 | S | D | A | E | N | R | R | D |
| 355 | S | D | A | E | N | R | T | N |
| 356 | S | D | A | E | N | R | T | D |
| 357 | S | D | A | E | N | S | R | N |
| 358 | S | D | A | E | N | S | R | D |
| 359 | S | D | A | E | N | S | T | N |
| 360 | S | D | A | E | N | S | T | D |
| 361 | S | D | A | E | S | R | R | N |
| 362 | S | D | A | E | S | R | R | D |
| 363 | S | D | A | E | S | R | T | N |
| 364 | S | D | A | E | S | R | T | D |
| 365 | S | D | A | E | S | S | R | N |
| 366 | S | D | A | E | S | S | R | D |
| 367 | S | D | A | E | S | S | T | N |
| 368 | S | D | A | E | S | S | T | D |
| 369 | S | D | A | Q | N | R | R | N |
| 370 | S | D | A | Q | N | R | R | D |
| 371 | S | D | A | Q | N | R | T | N |
| 372 | S | D | A | Q | N | R | T | D |
| 373 | S | D | A | Q | N | S | R | N |
| 374 | S | D | A | Q | N | S | R | D |
| 375 | S | D | A | Q | N | S | T | N |
| 376 | S | D | A | Q | N | S | T | D |
| 377 | S | D | A | Q | S | R | R | N |
| 378 | S | D | A | Q | S | R | R | D |
| 379 | S | D | A | Q | S | R | T | N |
| 380 | S | D | A | Q | S | R | T | D |
| 381 | S | D | A | Q | S | S | R | N |
| 382 | S | D | A | Q | S | S | R | D |
| 383 | S | D | A | Q | S | S | T | N |
| 384 | S | D | A | Q | S | S | T | D |

In particular examples, a capsid polypeptide of the present disclosure comprises a serine (S) at the position corresponding to position 482 and an aspartic acid (D) at the position corresponding to position 496 (e.g. C482S and N496D relative to SEQ ID NO:1); a serine (S) at the position corresponding to position 482 and a glutamine (Q) at the position corresponding to position 532 (e.g. C482S and K532Q); a serine (S) at the position corresponding to position 482, an aspartic acid (D) at the position corresponding to position 496 and a glutamine (Q) at the position corresponding to position 532 (e.g. C482S, N496D and K532Q relative to SEQ ID NO:1); a serine (S) at the position corresponding to position 482 (e.g. C482S relative to SEQ ID NO:1); a serine (S) at the position corresponding to position 482 and a serine (S) at the position corresponding to position 585 (e.g. C482S and R585S relative to SEQ ID NO:1); a serine (S) at the position corresponding to position 482, an alanine (A) at the position corresponding to position 503 and an aspartic acid (D) at the position corresponding to position 596 (e.g. C482S, T503A and N596D relative to SEQ ID NO:1); an alanine (A) at the position corresponding to position 503 and an aspartic acid (D) at the position corresponding to position 596 (e.g. T503A and N596D relative to SEQ ID NO:1); an aspartic acid (D) at the position corresponding to position 496, a glutamine (Q) at the position corresponding to position 532, a serine (S) at the position corresponding to position 585, a threonine (T) at the position corresponding to position 588 and an aspartic acid (D) at the position corresponding to position 596 (e.g. N496D, K532Q, R585S, R588T and N596D relative to SEQ ID NO:1); an aspartic acid (D) at the position corresponding to position 496, a threonine (T) at the position corresponding to position 588 and an aspartic acid (D) at the position corresponding to position 596 (e.g. N496D, R588T and N596D relative to SEQ ID NO:1); an alanine (A) at the position corresponding to position 503, a serine (S) at the position corresponding to position 585, a threonine (T) at the position corresponding to position 588 and an aspartic acid (D) at the position corresponding to position 596 (e.g. T503A, R585S, R588T and N596D relative to SEQ ID NO:1); or any combination of amino acid residues set forth in Table 6, below. In particular examples, the capsid polypeptides comprise the combination of amino acids at positions 482, 496, 503, 532, 582, 585, 588 and 596 as set forth in Table 8, below.

The capsid polypeptides may comprise 1, 2, 3, 4, 5, 6, 7, or 8 of these modifications (i.e. modifications at positions selected from among those corresponding to positions 482, 496, 503, 532, 582, 585, 588 and 596 of the AAV2 capsid polypeptide set forth in SEQ ID NO:1) in an AAV2 capsid polypeptide such as that set forth in SEQ ID NO:1, or in a polypeptide having at least or about 85%, 86%, 87%, 88%, 89%, 90%, 91%, 92%, 93%, 94%, 95%, 96%, 97%, 98%, or 99% sequence identity to the sequence set forth in SEQ ID NO:1. Thus, for example, the capsid polypeptide may comprise a sequence of amino acids set forth in SEQ ID NO:1 but further comprising one or more of the amino acid substitutions selected from among C482S, N496D, T503A, L532E, K532Q, N582S, R585S, R588T and N596D, such as in combinations shown in Table 3 above. In particular examples, the capsid polypeptide comprises a sequence of amino acids set forth in SEQ ID NO:1 but further comprising the amino acid substitutions C482S and N496D; C482S and K532Q; C482S, N496D and K532Q; just C482S; or any combination set forth in Table 6, below. In further examples, the capsid polypeptides comprise the combination of amino acids at positions 482, 496, 503, 532, 582, 585, 588 and 596 as set forth in Table 8, below. In other examples, the capsid polypeptides comprise a sequence of amino acids having at least or about 95% sequence identity to the polypeptide set forth in SEQ ID NO:1 and comprise one or more of the amino acid substitutions at positions corresponding to positions 482, 496, 503, 532, 582, 585, 588 and 596 of SEQ ID NO:1 as described above and shown in Table 3 (e.g. one or more of C482S, N496D, T503A, L532E, K532Q, N582S, R585S, R588T and N596D). In further examples, the Capsid polypeptides comprising such modifications include those comprising all or a portion of the VP1 protein (comprising amino acid residues corresponding to those at positions 1-735 of SEQ ID NO:1), VP2 protein (comprising amino acid residues corresponding to those at positions 138-735 of SEQ ID NO:1) and/or the VP3 protein (comprising amino acid residues corresponding to those at positions 203-735 of SEQ ID NO:1), including functional fragments thereof.

Capsid polypeptides of the present disclosure also include those comprising 1, 2, 3, 4, 5, or 6 modifications relative to the prototypic AAV3B capsid polypeptide set forth in SEQ ID NO:56 (also Genbank Accession AAB95452.1), wherein the modifications are at positions selected from among those corresponding to positions 476, 485, 488, 533, 594 and 598 of the prototypic AAV3B capsid polypeptide set forth in SEQ ID NO:56. The amino acid residue at any one or more of these positions may be substituted with any another amino acid residue. As discussed above, an AAV vector comprising such a capsid polypeptide typically exhibits improved in vivo transduction efficiency compared to an AAV vector comprising a prototypic AAV3B capsid polypeptide.

Exemplary modifications that may be present in a modified capsid polypeptide of the present disclosure include those resulting in a glutamine (Q) or asparagine (N) at position 476 (e.g. R476Q or R476N relative to SEQ ID NO:56); those resulting in a glutamine (Q) or asparagine (N) at position 485 (e.g. R485Q or R485N relative to SEQ ID NO:56); those resulting in a glutamine (Q) or asparagine (N) at position 488 (e.g. R488Q or R488N relative to SEQ ID NO:56); those resulting in a glutamic acid (E) or glutamine (Q) at position 533 (e.g. K533E or K533Q relative to SEQ ID NO:56); those resulting in a glutamic acid (E) or histidine (H) at position 598 (e.g. D598E or D598H relative to SEQ ID NO:56); and those resulting in a glycine (G), glutamic acid (E) or alanine (A) at position 594 (e.g. R594G, R594E or R594A relative to SEQ ID NO:56). In one embodiment, the modified capsid polypeptide comprises at least two modifications, with one resulting in glutamic acid (E) at position 594 (e.g. R594E) and one resulting in histidine (H) at position 598 (e.g. D598H).

The capsid polypeptides may comprise 1, 2, 3, 4, 5 or 6 of these modifications (i.e. modifications at positions selected from among those corresponding to positions 476, 485, 488, 533, 594 and 598 of the prototypic AAV3B capsid polypeptide set forth in SEQ ID NO:56) in an AAV3B capsid polypeptide such as that set forth in SEQ ID NO:56, or in a polypeptide having at least or about 85%, 86%, 87%, 88%, 89%, 90%, 91%, 92%, 93%, 94%, 95%, 96%, 97%, 98%, or 99% sequence identity to the sequence set forth in SEQ ID NO:56. Capsid polypeptides comprising such modifications include those comprising all or a portion of the VP1 protein (comprising amino acid residues corresponding to those at positions 1-736 of SEQ ID NO:56), VP2 protein (comprising amino acid residues corresponding to those at positions 138-736 of SEQ ID NO:56) and/or the VP3 protein (comprising amino acid residues corresponding to those at positions 203-736 of SEQ ID NO:56), including functional fragments thereof.

Capsid polypeptides of the present disclosure also include those comprising 1, 2, 3, 4 or 5 modifications relative to the prototypic AAV13 capsid polypeptide set forth in SEQ ID NO:55 (also Genbank Accession ABZ10812.1), wherein the modifications are at positions selected from among those corresponding to positions 482, 485, 525, 528 and 530 of the prototypic AAV13 capsid polypeptide set forth in SEQ ID NO:55. The amino acid residue at any one or more of these positions may be substituted with any another amino acid residue. As discussed above, an AAV vector comprising such a capsid polypeptide typically exhibits improved in vivo transduction efficiency compared to an AAV vector comprising a prototypic AAV13 capsid polypeptide.

Exemplary modifications that may be present in a capsid polypeptide of the present disclosure therefore include those resulting in a glutamine (Q) or asparagine (N) at position 482 (e.g. R482Q or R482N relative to SEQ ID NO:55); those resulting in a glutamine (Q) or asparagine (N) at position 485 (e.g. R485Q or R485N relative to SEQ ID NO:55); those resulting in a glutamic acid (E) or aspartic acid (D) at position 525 (e.g. K525E or K525Q relative to SEQ ID NO:55); those resulting in a glutamic acid (E) or aspartic acid (D) at position 528 (e.g. K528E or K528Q relative to SEQ ID NO:55); and those resulting in a glutamic acid (E) or aspartic acid (D) at position 530 (e.g. K530E or K530Q relative to SEQ ID NO:55).

The capsid polypeptides may comprise 1, 2, 3, 4 or 5 of these modifications (i.e. modifications at positions selected from among those corresponding to positions 482, 485, 525, 528 and 530 of the prototypic AAV13 capsid polypeptide set forth in SEQ ID NO:55) in an AAV13 capsid polypeptide such as that set forth in SEQ ID NO:55, or in a polypeptide having at least or about 85%, 86%, 87%, 88%, 89%, 90%, 91%, 92%, 93%, 94%, 95%, 96%, 97%, 98%, or 99% sequence identity to the sequence set forth in SEQ ID NO:55. Capsid polypeptides comprising such modifications include those comprising all or a portion of the VP1 protein (comprising amino acid residues corresponding to those at positions 1-733 of SEQ ID NO:55), VP2 protein (comprising amino acid residues corresponding to those at positions 137-733 of SEQ ID NO:55) and/or the VP3 protein (comprising amino acid residues corresponding to those at positions 202-733 of SEQ ID NO:55), including functional fragments thereof.

Also provided are nucleic acid molecules, including isolated nucleic acid molecules, encoding a capsid polypeptide of the disclosure. Exemplary nucleic acid molecules include those set forth in SEQ ID NOs:28-53 and 66-69 and those having at least or about 85%, 86%, 87%, 88%, 89%, 90%, 91%, 92%, 93%, 94%, 95%, 96%, 97%, 98%, or 99% sequence identity thereto.

5. Vectors

The present disclosure also provides vectors comprising a nucleic acid molecule that encodes a capsid polypeptide described herein, and vectors comprising a capsid polypeptide described herein. The vectors include nucleic acid vectors that comprise a nucleic acid molecule that encodes a capsid polypeptide described herein, and AAV vectors that have a capsid comprising a capsid polypeptide described herein.

5.1. Nucleic Acid Vectors

Vectors of the present disclosure include nucleic acid vectors that comprise a polynucleotide that encodes all or a portion of a capsid polypeptide described herein, e.g. that encodes a polypeptide comprising an amino acid sequence set forth in any one of SEQ ID NOs:2-27 and 62-64 or an amino acid sequence having at least or about 85%, 86%, 87%, 88%, 89%, 90%, 91%, 92%, 93%, 94%, 95%, 96%, 97%, 98% or 99% sequence identity to a sequence set forth in any one of SEQ ID NOs:2-27 and 62-64, or a fragment thereof (e.g. all or a portion of the VP2 or VP3 protein), as described above. The vectors can be episomal vectors (i.e., that do not integrate into the genome of a host cell), or can be vectors that integrate into the host cell genome. Exemplary vectors that comprise a nucleic acid molecule encoding a capsid polypeptide include, but are not limited to, plasmids, cosmids, transposons and artificial chromosomes. In particular examples, the vectors are plasmids.

Vectors, such as plasmids, suitable for use in bacterial, insect and mammalian cells are widely described and well-known in the art. Those skilled in the art would appreciate that vectors of the present disclosure may also contain additional sequences and elements useful for the replication of the vector in prokaryotic and/or eukaryotic cells, selection of the vector and the expression of a heterologous sequence in a variety of host cells. For example, the vectors of the present disclosure can include a prokaryotic replicon (that is, a sequence having the ability to direct autonomous replication and maintenance of the vector extra-chromosomally in a prokaryotic host cell, such as a bacterial host cell. Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors of the present disclosure can be constructed using known techniques, including, without limitation, the standard techniques of restriction endonuclease digestion, ligation, transformation, plasmid purification, in vitro or chemical synthesis of DNA, and DNA sequencing. The vectors of the present disclosure may be introduced into a host cell using any method known in the art. Accordingly, the present disclosure is also directed to host cells comprising a vector or nucleic acid described herein.

### 5.2. AAV Vectors

Provided herein are AAV vectors comprising a capsid polypeptide described herein, such as a polypeptide comprising all or a portion of a AAV capsid protein (e.g. a polypeptide comprising the amino acid sequence set forth in any one of SEQ ID NOs:2-27 and 62-65 or an amino acid sequence having at least or about 85%, 86%, 87%, 88%, 89%, 90%, 91%, 92%, 93%, 94%, 95%, 96%, 97%, 98% or 99% sequence identity to a sequence set forth in any one of SEQ ID NOs:2-27 and 62-65, or a fragment thereof (e.g. all or a portion of the VP2 or VP3 protein).

Methods for vectorizing a capsid protein are well known in the art and are described above (see e.g. section 1.2).

Typically, the AAV vectors of the present disclosure also comprise a heterologous coding sequence. The heterologous coding sequence may be operably linked to a promoter to facilitate expression of the sequence. Non-limiting examples of exemplary heterologous sequences and their construction within a vector are described above and are relevant for use in with AAV vectors of the present disclosure (see e.g. section 1.3). Additional elements, such as promoters, transcriptional enhancers, translational signals, transcriptional and translational termination signals, posttranscriptional regulatory elements etc. that can be present in an AAV vector of the present disclosure are known in the art (see e.g. section 1.3, above).

As will be appreciated by a skilled artisan, any method suitable for purifying AAV can be used in the embodiments described herein to purify the AAV vectors, and such methods are well known in the art. For example, the AAV vectors can be isolated and purified from packaging cells and/or the supernatant of the packaging cells. In some embodiments, the AAV is purified by separation method using a CsCl or iodixanol gradient centrifugation. In other embodiments, AAV is purified as described in US20020136710 using a solid support that includes a matrix to which an artificial receptor or receptor-like molecule that mediates AAV attachment is immobilized.

### 6. Host Cells

Also provided herein are host cells comprising a nucleic acid molecule or vector or of the present disclosure. In some instances, the host cells are used to amplify, replicate, package and/or purify a polynucleotide or vector. In other examples, the host cells are used to express a heterologous sequence, such as one packaged within AAV vector. Exemplary host cells include prokaryotic and eukaryotic cells. In some instances, the host cell is a mammalian host cell. It is well within the skill of a skilled artisan to select an appropriate host cell for the expression, amplification, replication, packaging and/or purification of a polynucleotide, vector or rAAV virion of the present disclosure. Exemplary mammalian host cells include, but are not limited to, HEK-293 cells, HeLa cells, Vero cells, HuH-7 cells, and HepG2 cells. In particular examples, the host cell is a hepatocyte or cell-line derived from a hepatocyte.

7. Compositions and Methods for Use

Also provided are compositions comprising the nucleic acid molecules, polypeptides and/or vectors of the present disclosure. In particular examples, provided are pharmaceutical compositions comprising the AAV vectors disclosed herein and a pharmaceutically acceptable carrier. The compositions can also comprise additional ingredients such as diluents, stabilizers, excipients, and adjuvants.

The carriers, diluents and adjuvants can include buffers such as phosphate, citrate, or other organic acids; antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

The AAV vectors of the present disclosure, and compositions containing the AAV vectors, may be used in methods for the introduction of a heterologous coding sequence into a host cell. Such methods involve contacting the host cell with the AAV vector. This may be performed in vitro, ex vivo or in vivo. In particular embodiments, the host cell is a hepatocyte (e.g. a human hepatocyte).

When the methods are performed ex vivo or in vivo, typically the introduction of the heterologous sequence into the host cell is for therapeutic purposes, whereby expression of the heterologous sequence results in the treatment of a disease or condition. Thus, the AAV vectors disclosed herein can be administered to a subject (e.g., a human) in need thereof, such as subject with a disease or condition amendable to treatment with a protein, peptide or polynucleotide encoded by a heterologous sequence described herein.

When used in vivo, titers of AAV vectors to be administered to a subject will vary depending on, for example, the particular recombinant virus, the disease or disorder to be treated, the mode of administration, the treatment goal, the individual to be treated, and the cell type(s) being targeted, and can be determined by methods well known to those skilled in the art. Although the exact dosage will be determined on an individual basis, in most cases, typically, recombinant viruses of the present disclosure can be administered to a subject at a dose of between 1×10¹⁰ genome copies of the recombinant virus per kg of the subject and 1×10¹⁴ genome copies per kg.

The route of the administration is not particularly limited. For example, a therapeutically effective amount of the AAV vector can be administered to the subject via, for example, intramuscular, intravaginal, intravenous, intraperitoneal, subcutaneous, epicutaneous, intradermal, rectal, intraocular, pulmonary, intracranial, intraosseous, oral, buccal, or nasal routes. The AAV vector can be administrated as a single dose or multiple doses, and at varying intervals.

Also provided are methods for producing an AAV vector described above and herein, i.e. one comprising a capsid polypeptide of the present disclosure. Such methods comprising culturing a host cell comprising a nucleic acid molecule encoding a capsid polypeptide the present disclosure, an AAV rep gene, a heterologous coding sequence flanked by AAV inverted terminal repeats, and helper functions for generating a productive AAV infection, under conditions suitable to facilitate assembly of an AAV vector comprising a capsid comprising a capsid polypeptide of the present disclosure, wherein the capsid encapsidates the heterologous coding sequence.

The disclosure of every patent, patent application, and publication cited herein is hereby incorporated herein by reference in its entirety.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

In order that the invention may be readily understood and put into practical effect, particular preferred embodiments will now be described by way of the following non-limiting examples.

### EXAMPLES

### Example 1. Materials and Methods

### Cell Line Culture Conditions

Human hepatoma HuH-7 cells and HEK293T cells (ATCC, CRL-1573) were maintained as monolayer cultures in Dulbecco's Modified Eagle's Medium (Sigma, Cat #D579) supplemented with 10% (v/v) Fetal Bovine Serum (Sigma, Cat #12006C), 100 μg/mL penicillin and 100 μg/mL streptomycin.

DNA Extraction from Human Liver Samples

DNA was extracted from liver tissue, using a QIAamp® Fast DNA Tissue Kit (Qiagen) according to the manufacturer's instructions, with the following modifications: (i) Frozen liver chunks (˜15 mg each) were homogenised in the digestion buffer using a plastic pestle and manual homogenisation to minimise DNA shearing. (ii) Digestion at 56° C. was carried out with intermittent agitation (5 sec/min) at 1,000 rpm for 30 minutes. (iii) Samples were triturated through a 22G needle to complete homogenisation. (iv) Samples were incubated at 56° C. for a further 90 minutes with intermittent agitation.

### PCR Amplification of Liver Derived AAV Capsid Sequences

PCR primers were designed to amplify full capsid sequences, by annealing forward primers in the rep gene and reverse primers in the 3′ITR, spanning the A/D region junction. Capsid sequences CMRI_01, CMRI_02, CMRI_03, CMRI_04, CMRI_05, CMRI_06, CMRI_07 and CMRI_08 were amplified with AAV2-based primers [Rep_mpx1_F: CGCAGACAGGTACCAAAACAAA (SEQ ID NO:11); 3UTR_mpx2_R: ACTCCATCACTAGGGGTTCC (SEQ ID NO:57), while capsid sequences CMRI_09 and CMRI_10 were amplified with primers displaying a broader AAV specificity [AAV(excl5)rep_fwd: GGTACCAAAACAAATGTTCTCGTCACG (SEQ ID NO:59) and a mix of AAV(prim)3ITR−D+G_R: GGAGTGGCCAACTCCATCACTAG (SEQ ID NO:60) and AAV(prim)3ITR-D+C_R: GGAGTGGGCAACTCCATCACTAG (SEQ ID NO:61)]. All amplification reactions employed Q5® Hot Start High-Fidelity DNA Polymerase (New England Biolabs) as per the manufacturer's standard conditions and 300 ng template DNA, using the following thermal cycling conditions: 98° C. for 30 sec; 40 cycles of 98° C. for 10 sec, 67° C. (for AAV-based primers) or 69° C. (for broader-specificity primers) for 30 sec, 72° C. for 2 min 30 sec; 72° C. for 10 min.

### Cloning and Sequencing of Liver-Derived AAV Capsid Sequences

Capsid amplicons were cloned using the StrataClone Blunt PCR Cloning Kit (Agilent) according to the manufacturer's instructions, and Sanger-sequenced at AGRF using standard M13 (−20) and M13 Reverse sequencing primers, as well as custom amplicon-internal sequencing primers.

### AAV Production: Iodixanol Gradient

Barcoded AAV genomes were packaged using 5×15 cm tissue culture dishes of HEK 293 cells (ATCC, cat #CRL-1573) seeded at 90-95% confluency. Briefly, 22.5 μg of the Adenovirus 5 helper plasmid, 7.5 μg of transfer vector and 7.5 μg of AAV-helper plasmid (encoding for rep2 and the specified capsid) were transfected per plate, by mixing the total 37.5 μg of DNA with 75 μg of polyethylenimine (PEI, MW 25000, Polysciences, Cat #23966-2) (DNA:PEI 1:2) in a total volume of 500 μL of OptiMEM media (Life Technologies, Cat #31985) per plate. The transfer vector (ITR-LSP1-eGFP—BC-WPRE-pA-ITR) contained AAV2 ITRs flanking eGFP under the control of the LSP1 promoter. A unique barcode (BC) for each transfer vector was fused to the eGFP gene so that each AAV vector having a specific capsid encapsulated a vector genome with a unique barcode. The transfer vector also contained the woodchuck hepatitis virus posttranscriptional regulatory element (WPRE) and a polyadenylation sequence downstream of the barcode.

Seventy-two hours post transfection cells were harvested and centrifuged at 2250×g for 15 min at 4° C. Following separation, supernatant and cells were treated separately. The supernatant was re-spun (2250×g, 15 min, 4° C.) and incubated for 3 hours on ice with ¼ volume of 40% Polyethylene Glycol (PEG) 8000 (Fisher Scientific, Cat #BP2331-1KG). After incubation, the solution was centrifuged (2250×g 4° C., 15 min) and PEG-pellet containing the AAV particles was resuspended in 2 mL of PBS Buffer. Separately, cell pellet was resuspended in 3 mL of PBS buffer and cells were lysed using three freeze/thaw cycles using dry ice+ethanol bath and 37° C. water bath. The resuspended PEG pellet and the cell lysate were then combined and incubated with Benzonase (200 U/mL) 1 hr at 37° C. 10% sodium deoxycholate (Sigma, Cat #D6750) was added to a final concentration of 0.5% and subsequently ¼ volume of 5 M NaCl was added to the final concentration of 1 M. Solution was incubated for 30 minutes at 37° C. (water bath) and spun 30 minutes at 2250×g at 4° C. The supernatant containing vector particles was collected into a 5 mL Eppendorf tube.

The four solutions required for iodixanol gradient (15%, 25%, 40% and 60%) were prepared: 15% iodixanol—19.8 mL PBS-MgKNa, 6.6 mL Optiprep™; 25% iodixanol—15.4 mL PBS-MgK, 11 mL Optiprep™, 66 μL phenol red stock (1:400 dilution); 40% iodixanol—7.3 mL PBS-MgK, 14.7 mL Optiprep™, 22 mL Total; and 60% iodixanol (100% Optiprep™)—22 mL Optiprep, 100 μL phenol red stock (1:220 dilution) (PBS-MgK: PBS with 1 mM MgCl₂ and 2.5 mM KCl); and PBS-MgKNa: 500 mL PBS-MgK plus 29.22 g NaCl). The 15% solution was added to the bottom of the Beckman tube (Beckman Coulter, Cat #361625) using a 10 mL syringe with a long 18-gauge metal cannula. Subsequent layers were added under previous layer (25%->6 mL; 40%->5 mL; 60%->5 mL) by extending the syringe needle to the bottom of the Beckman tube.

The recovered virus supernatant was then carefully added to the top of the gradient by slowly dripping the solution using a 5 mL pipette and avoiding disruption of the gradient layers. The remaining void of the Beckman tube was filled with balancing buffer (PBS, 10 mM Tris-HCl pH 8.5, 1 M NaCl) and tubes were balanced to within 0.01 g. The vector preparation(s) were centrifuged at 58,000×g in a Beckman Type 70Ti rotor for 2 hours and 10 minutes at 18° C. using a Beckman Coulter XPN ultracentrifuge set to acceleration speed 3 and deceleration speed 9 (lowest break). After centrifugation, tubes were carefully removed and mounted on a ring stand with a utility clamp inside tissue culture hood. Tube surface was cleaned with 70% ethanol and a 10 mL syringe with an 18-gauge needle was prepared for viral extraction upon needle insertion approximately 1.5 mm below the interface between the 40% and 60% gradient layers, with the bevel of the needle facing up. 3 to 5 mL of solution were then extracted, first with the beveled needle opening facing upwards and then facing downward, avoiding the collection of the visible protein-rich band at the 25/40% interface.

The 3-5 mL virus preparation were then filtered using a 0.22 μm syringe PES filter (Millipore, Cat #SLGP033RB) and mixed with Iodixanol Dialysis Buffer (PBS, 50 mM NaCl, 0.0001% Pluronic F68), to a total volume of 15 mL. Total volume was then moved to a 100K Amicon Ultra-15 Centrifuge Filter tube (Millipore, UFC910024) and centrifuged at 2250×g at 18° C. for 2-6 minutes to bring the volume down to desired volume, usually 200 to 500 μL. The vector was diluted with 15 mL of Iodixanol Dialysis Buffer and centrifuged again. This step was repeated two more times. Following the final spin, 200-500 μL of vector preparation were extracted from the Amicon Tube and moved to a 1.5 mL cryovial tube and stored at 4° C. for short-term storage or at −80° C. for long-term storage.

### Phenol/Chloroform/Isoamyl Alcohol DNA Extraction

200 μL of DNA material was mixed with equal volume of cold phenol/chloroform/isoamyl alcohol solution (Sigma-Aldrich, P2069) and tube was mixed vigorously for 1 minute and spun at 20,000×g for 5 minutes on a bench-top centrifuge. ˜180 μL of the top aqueous solution was removed and placed in a new 1.5 mL Eppendorf tube. Special care was taken to avoid the chloroform/isoamyl alcohol phase.

### Ethanol DNA Precipitation

3 M sodium acetate solution in water was added on the DNA solution to precipitate to a final concentration of 0.75 M. Solution was mixed thoroughly. 2.5× volume of 100% ethanol was added and mixed, the solution was left at −80° C. for 30 min and then spun for 20 min in a 4° C. centrifuge at 20,000×g. Supernatant was decanted carefully without disturbing the DNA pellet, which was washed by adding 300 μL of 80% ethanol and vortexed 3 times. Solution was subsequently spun again for 15 min in a 4° C. centrifuge at 20,000×g and supernatant was carefully removed. Pellet was air dried for 1-2 minutes at room temperature, and residual ethanol was removed with a P20 pipette. Pellet was re-suspended with desired volume of water.

### Library Gibson Assembly (GA) and Library Restriction Cloning

GA reaction was performed by mixing an equal volume of 2× Gibson Assembly Master Mix (NEB, Cat #E2611L) with 1 μmol of PCR amplified and DpnI treated receptor plasmid and 1 μmol of the recovered capsids, at 50° C. for 30 min. DNA was ethanol precipitated, and electroporated into SS320 electro-competent E. coli (Lucigen, Cat #60512-2). The total number of transformants was calculated by preparing and plating five 10-fold serial dilutions of the electroporated bacteria. The pool of transformants was grown overnight in 250 mL Luria-Bertani media supplemented with trimethoprim (Sigma-Aldrich, T7883) (final concentration of 10 μg/mL). Total plasmids were purified with an EndoFree Maxiprep Kit (QIAGEN, Cat #12362) as per manufacturer's instructions and subsequently digested overnight with SwaI and NsiI. 1.4 μg of insert was ligated at 16° C. using T4 DNA ligase (NEB, Cat #M0202) for 16 hours into 1 μg of the recipient plasmid digested with SwaI/NsiI. Ligation reactions were concentrated using ethanol precipitation, electroporated into SS320 electro-competent cells and grown as described above. Total plasmids were purified with an EndoFree Maxiprep Kit (QIAGEN, Cat #12362).

### DNA Micro-Dialysis

When desalting the Gibson Assembly solution was required in order to avoid arcing during electroporation reaction, a DNA micro-dialysis was performed using 0.025 μm Millipore desalting paper (Merck, Cat #VSWP01300). Briefly, the paper was placed on top of a water in 10 cm TC dish and the assembly solution was pipetted on top of the filter and left for one hour at room temperature and carefully recovered with a micropipette.

Repopulation of Murine Liver with Human Hepatic Cells (hFRG)

All the FRG mice described were xenotransplanted at the Children's Medical Research Institute BioResources facility. Procedures complied with the stringent guidelines set by the National Health and Medical Research Council (NHMRC). Mice undergoing transplantation were 8-12 weeks of age, Fah^{-/-}/Rag2^{-/-}/Il2rg^{-/-}. To enhance engraftment, recipient mice were injected with 5×10⁹ pfu Adenovirus5-urokinase in 100 μL PBS via the tail vein 24-48 hours prior to transplantation. Hepatocytes to be engrafted were thawed immediately prior to intrasplenic transplant, washed with cold DMEM (no FBS), centrifuged and resuspended in 100 μL aliquots at a concentration of 1×10⁷ cells/mL (total of 1×10⁶ cells/vial). For intrasplenic injection mice were anaesthetised by inhalation of vaporised isoflurane, delivered by the stinger research anaesthetic gas machine (Advanced Anaesthesia Specialist, Cat #2848). Mice received analgesia prior to surgery (buprenorphine, 0.03 mg/kg). A one-centimetre incision was made in the left flank of the animal and the spleen partially extracted through the incision. The donor hepatocytes were slowly injected using a 29-gauge insulin syringe into the inferior pole of the immobilised spleen. At completion, the needle was slowly removed and pressure was applied to the injection site until haemostasis was stablished. The abdominal wall was closed, swabbed with betadine. Mice received antibiotics in drinking water (Baytril, 0.17 mg/mL, Bayer). In order for the foreign hepatocytes to establish and expand in the livers of recipient mice, 2-(2-nitro-4-trifluoro-methylbenzoyl)-1,3-cyclohexanedione (NTBC) was slowly withdrawn from the drinking water post-transplant before a regime of cycling on and off NTBC was established. Specifically, immediately after transplant NTBC was reduced to 0.8 μg/mL for 2-3 days, 0.4 μg/mL for 2-3 days, 0.2 μg/mL for 2-3 days, 0 μg/mL until day 10-14 post-surgery before return to 8 μg/mL NTBC and subsequent cycling (21 days OFF NTBC, 5 days ON NTBC, to 0.8 μg/mL). To gauge the level of engraftment blood was collected via tail bleeds and the level of human serum albumin established by ELISA.

### Mouse Liver Perfusion

Solutions 0-4 (Solution 0:25 mL of HBSS−/−; Solution 1:25 mL of HBSS−/−; Solution 2:25 mL of HBSS−/−0.5 mM EDTA; Solution 3:25 mL of HBSS−/−; Solution 4:25 mL of HBSS−/−5 mM CaCl, 0.05% Collagenase IV, 0.01% DNaseI) (where HBSS−/− contains no calcium chloride, no magnesium chloride and no magnesium sulfate, Gibco Cat #14175-095) were warmed to 37° C. in a heating block. Perfusion pump was set up and primed with Solution 0 at high speed. Mice were anaesthetised and culled by isofluorane overdose. The abdomen was opened, and guts were moved to expose inferior vena cava (IVC). A needle (22G) was inserted the inferior vena cava and tubing was then attached to catheter. The portal vein was cut and the pump speed was increased. Liver was then perfused with Solutions 1 through 4. The liver was then transferred to a specimen jar, minced and rinsed with 25 mL of cold DMEM media supplemented with 10% FBS. The mix was then filtered through a 100 μm nylon filter into a 50 mL Falcon tube.

AAV Replication-Competent Selection in hFRG Mice

For the first round of selection, 1×10¹² vector genomes of the library were injected intraperitoneally (i.p.) into an hFRG mouse. Twenty-four hours post-injection, 8 μL of human Adenovirus 5 (ATCC, VR-1516) were injected i.p. to support AAV replication in human hepatocytes. Liver was harvested 72 hrs later, weighed and homogenised before 400 μL of PBS were added to 200 mg of homogenised liver. Three rounds of freeze/thaw cycles were carried out in dry ice+ethanol/water bath (37° C.). Liver lysate was further homogenised with a tissue homogenizer and spun at top speed on a benchtop centrifuge at 4° C. for 30 min. Cell debris was discarded and supernatant was incubated at 65° C. in order to inactivate hAd5. For the second round of selection, 200 μL of liver lysate was injected i.p into a hFRG mouse and the process described above repeated. Subsequent rounds of selection were performed as required.

### AAV Replication-Competent Selection in HUH-7 Cells

A total of 1×10⁵ cells was seeded per well in two 24-well tissue culture dishes 16 hours prior to infection with AAV library. Four 10-fold dilutions of the AAV library were added to the media in duplicate plates. Cells were washed with 1 mL of PBS 24 hrs after infection and 500p1 of fresh media was added to each well. To facilitate AAV library replication, wild-type human Adenovirus 5 (hAd5) (ATCC, Cat #VR-1516) was added at a multiplicity of infection (MOI) of 0.42 (based on 7 day TCID50) to all the wells in one of the plates. The plate without hAd5 served as a qPCR control. Cells were harvested 72 hrs after hAd5 infection and lysed by three freeze/thaw cycles. Cellular debris was removed by centrifugation (10 min, 1,000×g, 4° C.) and supernatant containing AAV particles analysed by qPCR. To do so, 8 μL of the matched library dilutions (±hAd5) were treated with DNaseI (NEB Cat #M030S) at 37° C. for 1 hr and the enzyme was heat inactivated at 75° C. for 10 min. qPCR with rep2-specific primers was used after each round to confirm library replication and to select the library dilution to be moved into subsequent rounds of selection. At each step the highest library dilution that resulted in no less than a 2 log increase in AAV signal was selected to minimize cross-packaging of multiple vectors in single packaging cells and thus to increase the stringency of the selection process. The library dilution selected for subsequent rounds of amplification was incubated at 65° C. for 30 min to inactivate hAd5. Usually up to 5 iterative selections were performed using the above described conditions.

RNA Isolation and cDNA Synthesis

Total RNA from cell pellet was isolated with the Direct-zol RNA kit (Zymo Research, Cat #R2060) following manufacturer's instructions. RNA was incubated with DNase I (RNAse-free, NEB, Cat #M0303S) for 1 hour at 37° C. to remove any contaminating DNA, including AAV DNA. cDNA first-strand synthesis was performed using SuperScript IV First-strand synthesis system (Thermo Fisher, Cat #18091050), following manufacturer's instructions.

### Next Generation Sequencing

PCR products were amplified using Q5® High-fidelity DNA polymerase (NEB, Cat #M0491S) and Illumina sequenced using the Standard Amplicon Sequencing offered by Genewiz® (Guangzhou, China facility). Adapter ligation libraries were prepared at the sequencing facility and Illumina 2×150 bp/Illumina 2×300 bp platforms were used as indicated in the main text.

### DNA Sanger Sequencing

Individual clones were Sanger sequenced at the Garvan Molecular Genetics Facility (Garvan Institute, Sydney).

### Processing of Frozen Liver Sections

Liver tissue was cut into small ˜3 mm thick pieces and fixed in 4% Paraformaldehyde Solution (PFA) at room temperature for 4-6 hours. Tissue was then washed in PBS and cryoprotected in 10% sucrose (in PBS) for 2 hours, 20% sucrose for 2 hours and then 30% sucrose overnight at 4° C. Sucrose was drained from samples and placed into a labelled cryomold of the appropriate size containing a small amount of Tissue-Tek O.C.T. compound (ProSciTech, IA018). The cryomold was then top up with O.C.T and placed into a plastic beaker containing pre-cooled isopentane standing in a recipient of liquid nitrogen. Frozen samples were wrapped in foil and stored at −80° C. 5 μM liver sections were cut using the cryostat.

### GAPDH Antibody Staining for Liver Sections

Primary antibody: rabbit monoclonal to GAPDH conjugated with Alexa Fluor 647 (Abcam, #ab215227). Dilution 1:100-1,000. Liver slides were washed in PBS for 5 minutes and sections were marked with wax pen and subsequently permeabilized with 0.1% Triton X-100 in PBS for 15 minutes at room temperature. Sections were washed with 1×PBS, 2×PBS/2% FCS for 5 minutes each and subsequently blocked for at least 30 minutes in donkey serum blocking buffer [10% donkey serum in PBS, Sigma Cat #D9663). Primary antibody, anti-human GAPDH was prepared (1:650 in PBS) and the slide was incubated at room temperature for one hour, subsequently washed three times with 0.1% Tween-20/PBS (5 minutes each) and ten PBS (5 minutes). The slides were dried and after addition of coverslip left in the dark for 10 minutes.

### In Vitro Transduction Analysis

Cells were plated and transduced using concentrations and MOIs as indicated, with and without the addition of heparin sodium from porcine intestinal mucosa (Sigma, Cat #H3393). Routinely, 72 hours after AAV transduction cells were harvested using TripLE™ Express enzyme reagent and resuspended in 200 μL of FACS buffer. eGFP/mCherry expression was quantified following flow cytometry with the data being analysed using FlowJo 7.6.1 software.

### Heparin Binding Assay

The heparin binding assay was performed using the ÄKTA™ pure chromatography system (GE Healthcare) with a 1 mL HiTrap® Heparin HP column (GE Healthcare, product number 17-0406-01; bed size: 7 mm×25 mm; bed volume: 1 mL). The HiTrap® Heparin HP column is prepacked with Heparin Sepharose® High Performance, in which the heparin has been coupled to the Sepharose® High Performance base matrix via the N-hydroxysuccinamide coupling method.

The column was run at a flow rate of 1 mL/minute. The column was equilibrated with 3 column volumes (CV) of buffer B (PBS+1 M NaCl, 1 mM MgCl₂ and 2.5 mM KCl), then 5 CV of buffer A (40 mM NaCl, 1 mM MgCl₂ and 2.5 mM KCl, 10 mM Phopshate, pH=7.4). The sample (3×10¹² vg of AAV diluted to 40 mM total [NaCl] and concentrated to 150 μL) was applied with a 2 mL sample loop, which was emptied with 4 mL buffer A. Fractions of 0.25 mL were then collected before the column was washed with 20 CV buffer A. Following the wash, the first 3 CV were collected in 0.25 mL fractions, with the following 7 CV being collected in a 50 mL tube. The final 10 CV of wash was collected in another 50 mL tube. Elution of the column was performed with a linear gradient of 0-100% buffer B over 10 CV, during which 0.25 mL fractions were collected. The column was then regenerated with 3 CV 5 M NaCl (collected in 0.25 mL fractions) before being washed with 5 CV water.

To analyse the vector distribution, qPCR was performed on various fractions using primers specific for the eGFP gene in the vector genome. The NaCl concentration ([NaCl]) at the elution peak maxima (as measured by A280 readings) was interpolated against a standard curve of conductivity versus [NaCl]. Silver staining was also performed to visualise the vector distribution in the various fractions.

### Example 2. AAV Capsid Polypeptides Identified from DNA-Family Shuffled Library

Seven AAV capsid polypeptides were identified and isolated after four rounds of selective replication in vivo (using hFRG mice) of a DNA-family shuffled library generated by mixing capsids genes from AAV serotypes 1 to 12, marsupial AAV (mAAV) and AAV-EVE (endogenous viral element). The seven capsid polypeptides included CMRI_11 (SEQ ID NO:12), CMRI_12 (SEQ ID NO:13), CMRI_13 (SEQ ID NO:14), CMRI_14 (SEQ ID NO:15), CMRI_15 (SEQ ID NO:16), CMRI_16 (SEQ ID NO:17) and CMRI_17 (SEQ ID NO:18).

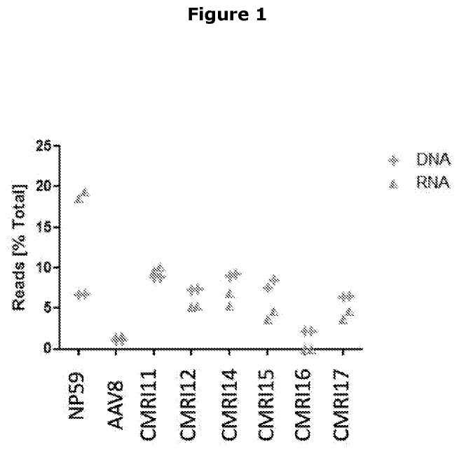

CMRI_11, CMRI_12, CMRI_14, CMRI_15, CMRI_16 and CMRI_17 were then vectorized for subsequent in vivo functional analysis using hFRG mice. Barcoded AAV transgenes (ITR—Liver Specific Promoter—GFP—Barcode—WPRE—pA) were packaged to the newly-identified capsids, as well as AAV8 and NP59 (Paulk et al., Mol Ther. 2018, 26(1):289-303), as described above. One week after injection of the mouse with each of the vectors, the chimeric liver was perfused and human and murine hepatocytes were single cell sorted. DNA and RNA were recovered from the human population of hepatocytes and Next Generation Sequencing (NGS) of the barcoded transgene was performed. As shown in FIG. 1, all of the newly-identified vectors except CMRI_16 physically transduced human hepatocytes in vivo at the same levels as NP59 based on DNA reads. Functional transduction (as reflected by RNA reads) appeared lower than NP59.

### Example 3. AAV Capsid Polypeptide Identified Following AAV2 Replication In Vivo

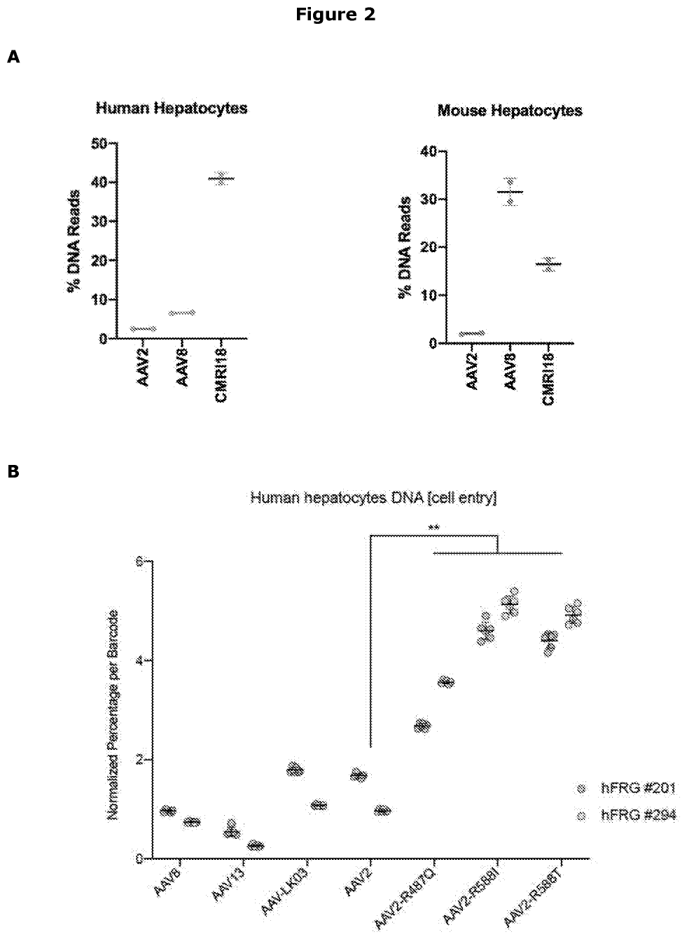

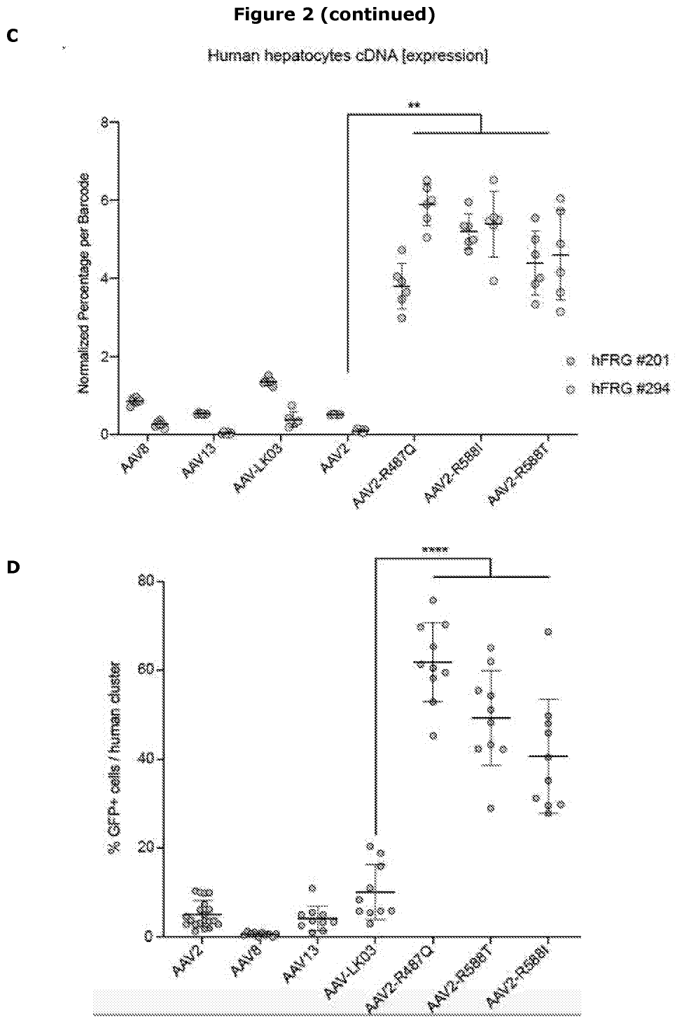

The AAV capsid polypeptide CMRI18 (containing the mutation R588I in the prototypic AAV2 background; SEQ ID NO:9) was isolated after four rounds of iterative replication-competent selection of a prototypic AAV2 construct in an hFRG mouse. CMRI_18 was vectorised as described above and injected intravenously into an hFRG mouse with AAV2 and AAV8, and two weeks later the liver was collected and analysed using Next Generation Sequencing to determine AAV-derived DNA levels. It was observed that CMRI18 preferentially transduces human hepatocytes, but also transduces murine hepatocytes (FIG. 2A). The in vivo transduction efficiency of CMRI_18 was again assessed in a subsequent study, along with AAV2, AAV8, AAV13, AAV-LK03 and other variants described in Example 4, and was observed to be significantly more effective at transducing human hepatocytes than AAV2 and AAV-LK03 (FIG. 2B-D, where CMRI18 is referred to as AAV2-R588I).

### Example 4. Other AAV Capsid Polypeptides

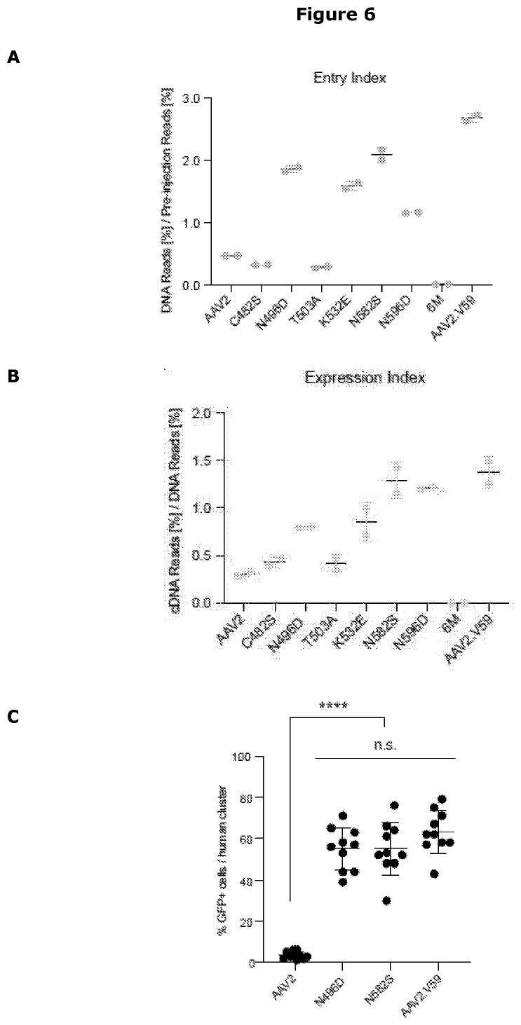

Three AAV capsid polypeptides were identified using a diverse capsid library. These capsids included CMRI_19 (containing the mutation N496D; SEQ ID NO:20), CMRI_20 (containing the mutation K532Q; SEQ ID NO:21) and CMRI_21 (containing the mutation N582S; SEQ ID NO:22). CMRI_19 and CMRI_21 were vectorized (resulting vectors sometimes referred to as AAV2_N496D and AAV2_N582S, respectively), and their function assessed in vivo using hFRG mice as described above. Each of these vectors transduced hepatocytes more effectively than the prototypic AAV2 vector (approximately 70-80 times better than AAV2; FIG. 6).

Other AAV capsid polypeptides provided herein include CMRI_27 (containing the mutation R588T in the prototypic AAV2 background; SEQ ID NO:62), CMRI_28 (containing the mutation R487Q in the prototypic AAV2 background; SEQ ID NO:63), CMRI_29 (containing the mutation K532E in the prototypic AAV2 background; SEQ ID NO:64), and CMRI_30 (containing the mutations T503A+N596D in the prototypic AAV2 background; SEQ ID NO:62). As shown in FIG. 2B-D, CMRI_27 (AAV2-R588T) and CMRI_28 (AAV2-R487Q), like CMRI_18 (AAV2-R588I), exhibited improved in vivo transduction of human hepatocytes compared to AAV2 and AAV-LK03.

Additional capsids produced include one containing the mutation C482S in the prototypic AAV2 background, one containing the mutation T503A in the prototypic AAV2 background, and one containing the mutation N596D in the prototypic AAV2 background (see FIG. 6, also).

### Example 5. AAV Capsid Polypeptides Isolated from Human Liver

Ten AAV capsid polypeptides were identified and isolated from human liver samples: CMRI_01 (SEQ ID NO:2), CMRI_02 (SEQ ID NO:3), CMRI_03 (SEQ ID NO:4), CMRI_04 (SEQ ID NO:5), CMRI_05 (SEQ ID NO:6), CMRI_06 (SEQ ID NO:7), CMRI_07 (SEQ ID NO:8), CMRI_08 (SEQ ID NO:9), CMRI_09 (SEQ ID NO:10), and CMRI_10 (SEQ ID NO:11). Sequence alignments and identity analysis indicated that CMRI_02 and CMRI_04 are AAV2 isolates, while CMRI_01, CMRI_03, CMRI_05, CMRI_06 and CMRI_07 appear to be AAV13/AAV2 hybrids. CMRI_01-08 were identified using the Rep_mpx1_F and 3UTR_mpx2_R primers, while CMRI_09 and CMRI_10 were identified using the AAV(excl5)rep_fwd, AAV(prim)3ITR-D+G_R and AAV(prim)3ITR−D+C_R primers. Functional analysis of CMRI_01-CMRI_07 is described in Example 13, below.

Five AAV additional capsid polypeptides were identified and isolated from human liver using the AAV(excl5)rep_fwd, AAV(prim)3ITR-D+G_R and AAV(prim)3ITR-D+C_R primers. These included CMRI_22 (SEQ ID NO:23), CMRI_23 (SEQ ID NO:24), CMRI_24 (SEQ ID NO:25), CMRI_25 (SEQ ID NO:26) and CMRI_26 (SEQ ID NO:27).

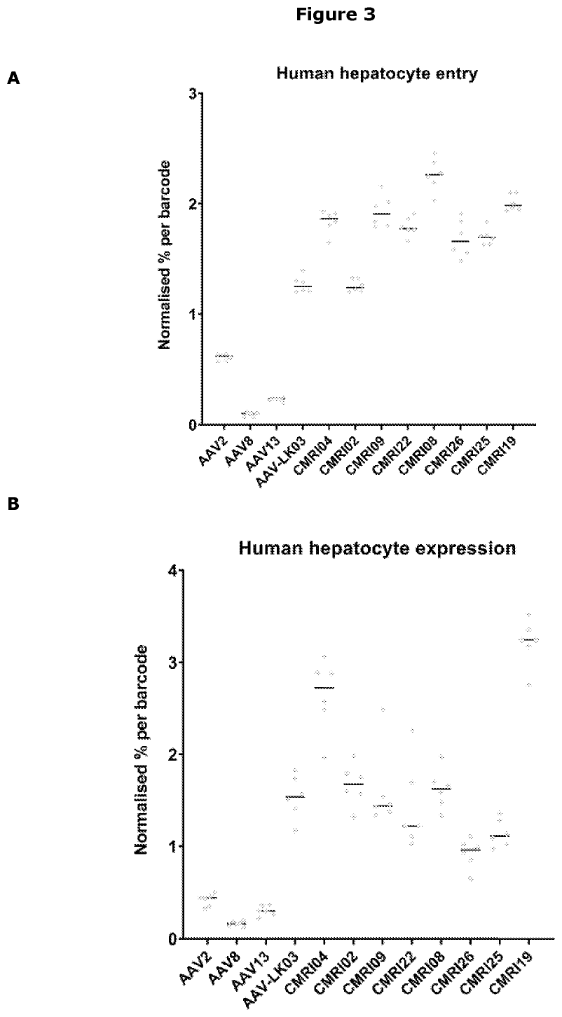

CMRI_08, CMRI_09, CMRI_19, CMRI_22, CMRI_25, and CMRI_26 were vectorized for in vivo functional analysis using an hFRG mouse. Barcoded AAV transgenes (ITR—Liver Specific Promoter—GFP—Barcode—WPRE—pA—ITR) were packaged to the newly-identified capsids. Vectors based on CMRI_02, CMRI_04, AAV2, AAV8, AAV13 and LK03 capsids were also used, and all vectors were injected into an hFRG mouse. One week after injection of the mouse with each of the vectors, the chimeric liver was perfused and human and murine hepatocytes were single cell sorted. DNA and RNA were recovered from the human population of hepatocytes and Next Generation Sequencing (NGS) of the barcoded transgene was performed. As shown in FIG. 3, all newly-described vectors exhibited improved physical transduction (i.e. DNA entry) and functional transduction (i.e. expression) compared to AAV2, and CMRI_19 in particular exhibited improved transduction.

### Example 6. Identification of Capsid Residues Important for In Vivo Transduction

Heparan sulfate proteoglycans mediate both AAV attachment to and infection of target cells. While there is substantial evidence that liver is a major target organ for at least AAV2, AAV3B and AAV13, including the identification of liver-specific enhancer-promoter activity in the 3′ untranslated region of prototypic AAV2 (Logan et al. Nat Genet. 2017 August; 49(8):1267-1273), when tested in vivo on human hepatocytes (FRG mouse xenografted liver model), AAV2 exhibits very poor transduction. The low in vivo transduction of primary human hepatocytes is supported by what was observed in a clinical trial for haemophilia B in which prototypic AAV2 vector was used and led to low expression of therapeutic protein (Manno et al. Nat Med. 2006 March; 12(3):342-7).

Three highly, in vivo hepatotropic human AAV vectors NP59, NP40 and NP84 were recently identified (Paulk et al., Mol Ther. 2018 Jan. 3; 26(1):289-303). Interestingly, the capsid of NP59 differs only by 11 amino acids compared to the prototypic AAV2 capsid (SEQ ID NO:1): at positions 162, 168, 179, 180, 190, 233, 235, 310, 312, 503 and 596. While the amino acid residues at positions 162, 168, 179, 180, 190, 233, 235, 310 and 312 of NP59 were the same as those found at the corresponding positions of AAV1, AAV6 and/or AAV3, the amino acid residues at positions 503 and 596 appeared to be novel mutations (T503A and N596D).

Studies were performed to identify which of the 11 amino acids were important in transduction efficiency. Two independent sets of combinatorial experiments were performed and the mutations T503A and N596D in NP59 (relative to prototypic AAV2, SEQ ID NO:1) were identified as being key for the improved in vivo transduction of NP59 (data not shown). In one study, a binary capsid library (AAV2^{Lib2048}) containing all possible permutations (n=2¹¹=2048) of AAV2 and NP59-specific residues at the eleven variable positions. To prevent formation of additional random changes in the cap gene due to replication-driven in vivo evolution, the AAV2^{Lib2048} library was cloned into a replication-incompetent selection platform encoding an LSP1-eGFP reporter cassette. This library platform design allows selection based on transgene expression (functional transduction) and thus to identify variants that, similar to AAV-NP59, could functionally transduce human hepatocytes with high efficiency. Analysis of the starting library using Illumina next-generation sequencing (NGS) confirmed the intended binary composition at each position, while full-length cap sequencing of n=27 randomly selected clones confirmed the binomial distribution of the AAV2^{Lib2048} library. The library underwent four rounds of iterative in vivo selection on primary human hepatocytes in hFRG mice. Illumina sequencing of the capsid regions containing the 11 key positions after Rounds 2 and 4 revealed a positive selection of NP59 residues at positions 503 (T503A) and 596 (N596D). The remaining positions showed preference for residues from AAV2 (amino acids 162, 168, 190, 235), or showed no strong preference (amino acids 179, 180, 233, 310). The residue at 312 showed a mild preference in favor of NP59. Importantly, further analysis revealed that clones harboring the double mutation (T503A+N596D) showed the strongest fold enrichment, suggesting a synergistic functional effect between both amino acids. These results, and the results of the other combinatorial study (data not shown) point at T503A and N596D as the key amino acid changes driving the observed in vivo differences between AAV2 and AAV2.V59. Interestingly, these two amino acids are the only ones out of the 11 differences between NP59 and AAV2 that are situated on the exterior of the capsid, specifically on the three-fold protrusion. Of additional note, vector yield analysis revealed that all of the variants in the combinatorial studies carrying T503A and N596D mutations showed significantly higher yields during production than vectors containing AAV2 residues at these positions (data not shown).

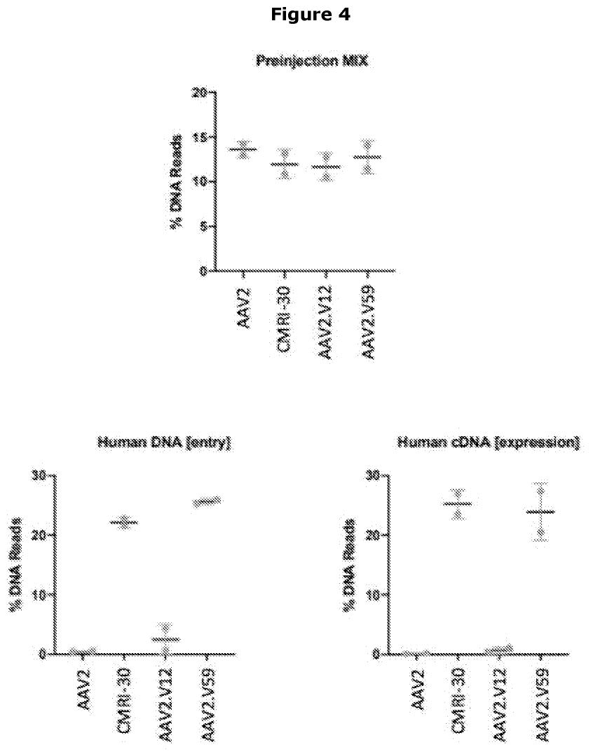

The T503A and N596D mutations were introduced into the AAV2 capsid polypeptide, and the resulting polypeptide (CMRI_30) was vectorised. These same mutations were also reversed in the NP59 capsid polypeptide set forth in SEQ ID NO:6, and the resulting polypeptide, which now contained mutations only at positions 162, 168, 179, 180, 190, 233, 235, 310 and 312 relative to AAV2, was vectorised to produce AAV2.V12. As demonstrated in FIG. 4, the presence of T503A and N596D in CMRI_30 resulted in improved in vivo transduction of hepatocytes in hFRG mice compared to AAV2, while reversion of the mutations in an NP59 capsid backbone (i.e. AAV2.V12), abrogated transduction to levels equivalent to that of AAV2.

Interestingly, the relative transduction efficiencies of AAV2 and NP59 in vivo are reversed in vitro, whereby AAV2 was far more efficient than NP59 at transducing hepatocyte derived cell lines, such as HuH-7 cells, or primary human hepatocytes, in vitro (data not shown).

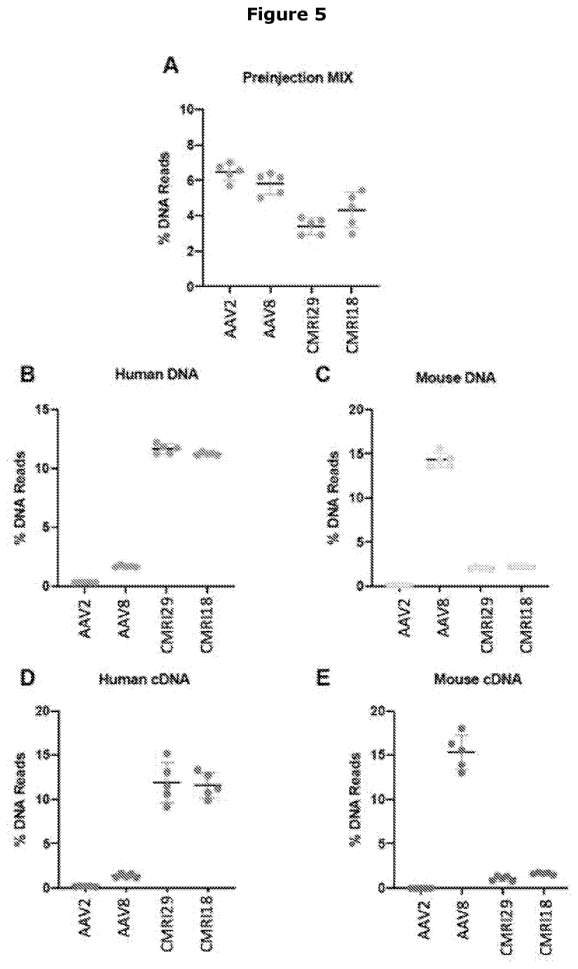

The highly human hepatotropic NP40 vector also differs by 11 amino acids from the prototypic AAV2, at positions 14, 162, 168, 179, 180, 190, 233, 235, 310, 312 and 532. Thus, NP40 does not contain T503A or N596D, which are the main drivers of NP59 in vivo transduction performance. Amino acid residues at positions 14, 162, 168, 179, 180, 190, 233, 235, 310 and 312 of NP40 were the same as those found at the corresponding positions of AAV1, AAV3B, AAV6 and/or AAV9. In contrast, the amino acid at position 535 of NP40 appears to be a new mutation: K532E. This mutation directly affects one of the five AAV2 residues known to mediate binding to heparan sulfate. To assess the effect that this mutation has on the performance of AAV2 in vivo, a novel variant of AAV2 having the K532E mutation compared to SEQ ID NO:1 (CMRI_29) was generated and its in vivo transduction performance compared with AAV2, AAV8 and CMRI_18 (i.e. AAV2 containing the an R588I mutation) using the hFRG mouse model. As shown in FIG. 5, introduction of the K532E or the R588I mutation (CMRI_29 and CMRI_18, respectively) substantially enhanced in vivo transduction compared to that observed with AAV2.

### Example 7. Correlation Between Heparin Binding Capabilities and In Vivo Transduction

The fact that two different combinations of amino acid substitutions (K532E and T503A/N596D) with similar characteristics (introduction of a negative charge on the 3-fold spike) led to similar improvement of vector function in vivo suggested the possibility that overall capsid charge could be playing an important role in in vivo transduction. Importantly, attachment of AAV2 to HSPG is mainly mediated by electrostatic interactions. Therefore, introduction of negative charges on the region mediating AAV2-HSPG binding could, in theory, reduce AAV2 affinity to HSPG.

The binding affinity of the AAV2 variants AAV2_T503A+N596D (CMRI_30) and AAV2_K532E (CMRI_29) on a heparin column was therefore assessed. Heparin is an HSPG analogue, and thus affinity for heparin is indicative of affinity for HSPG. It was observed that AAV2 had a stronger affinity for HSPG than each of the variants ([NaCl] at elution peak maxima (mM): AAV2=454 mM; CMRI_30=380 mM; CMRI_29=281 mM), suggesting that a modulation of the HSPG binding affinity might be beneficial for in vivo performance. Without being bound by theory, it is postulated that HSPG molecules present on the extracellular matrix of liver and other organs might be sequestering AAV2 given its very high binding affinity for HSPG, reducing the ability of AAV2 to effectively transduce the hepatocytes. A decrease in this affinity may enable the vectors to “escape” sequestration and transduce hepatocytes. This situation can be contrasted to the in vitro setting, where there is no extracellular matrix and thus the stronger affinity for HSPG shown by AAV2 results in better transduction.

### Example 8. In Vivo Adaptation of Prototypical AAV2

It was investigated whether it was possible to “re-adapt” the prototypical AAV2 on primary human hepatocytes in order to improve in vivo function. To this end, a replication competent version of AAV2 (RC-AAV2, ITR2-rep2-cap2-ITR2) was used to perform an iterative in vivo adaptation experiment on primary human hepatocytes in xenograft FRG mice in the presence of wtAd5. Naïve FRG mice were used as a negative control. Sequence analysis of n=24 random full-length capsids recovered after the first round of the in vivo adaptation experiment revealed substantial enrichment (16.6% of the population) of an AAV2 clone harbouring an R588I modification, affecting one of the key arginine residues involved in HSPG binding. Interestingly, 12.0% of clones analysed after round 2 harboured an R487Q mutation. While this clone was selected against, or outcompeted by R588I, in the subsequent rounds of selection, due to the fact that the R487Q affected another key arginine residue involved in heparin binding, this variant was included in subsequent analysis.

To gain a better understanding of the kinetics of the adaptive genetic shift, Illumina high-throughput NGS sequencing of the 150-bp region surrounding the affected positions 487 and 588 was performed in the initial virus stocks and after each round of in vivo selection. It was observed that the R588 present in AAV2 was rapidly replaced with an isoleucine (R588I) within two rounds of selection. Detailed analysis of the pre-adapted RC-AAV2 preparation suggests that the identified variants resulted from random mutagenesis events that gave rise to AAV2 variants with significantly improved tropism for primary human hepatocytes. Supporting this hypothesis, a third variant, R588T, appeared after the second round of selection, and accounted for 1.37 of the total reads. However, AAV2-R588T was no longer detectable after the subsequent round of selection (data not shown).

To further validate the hypothesis that the observed variants resulted from random mutagenesis events occurring during viral replication, the in vivo adaptation study was repeated using a new preparation of RC-AAV2. It was observed that during the second independent in vivo adaptation experiment, AAV2-R588T rapidly took over the population, and accounted for 88% of the clones following the second round of selection (data not shown). The fact that AAV2-R588I was outcompeted by AAV2-R588T, a variant that functionally is indistinguishable from AAV2-R588I, supports the hypothesis that the replication based in vivo evolution is a stochastic process and that the first variant to appear with seemingly increased fitness rapidly takes over the culture. It is important to note that both R588I and R588T mutations correspond to a single nucleotide change, from the original AGA codon encoding for arginine to either ATA (Ile) or ACA (Thr), consistent with the possibility that wtAAV has evolved an intrinsic mechanism to allow for rapid adaptation to changing conditions following minimal change at the DNA level. It can thus be further postulated that during an AAV infection, individual novel clones arising from random mutagenesis may be uniquely adapted to infect other tissues, allowing for simultaneous infections of multiple organs and formation of natural viral reservoirs.

The interaction between AAV2's 3-fold protrusions and HSPG has been reported to be mediated mainly by interactions of the sugar sulfates with positively charged basic residues located on the face of the 3-fold protrusions. The three mutations, R487Q and R588I/T, disrupt and reduce the basic charge of AAV2 (data not shown), and would therefore reduce the strength of the AAV2-HSPG interaction. To evaluate functionality of the AAV variants, the three clones, referred to as AAV2-R588I (CMRI_18), AAV2-R588T (CMRI_27), and AAV2-R487Q (CMRI_28), were vectorised and used to package the AAV-LSP-eGFP—BC-WPRE-BGHpA cassette. To more comprehensively understand the effect of the mutations on vector function, the three variants were tested in vitro on HuH-7 cells and in vivo on primary human hepatocytes in humanised FRG mice. As expected, all variants showed reduced transduction of HuH-7 at the MOIs tested when compared to the prototypical AAV2, performance that was not affected by soluble heparin at the concentration tested (data not shown). In striking contrast to the in vitro results, all three variants were found to outperform AAV2 and the human tropic bioengineered capsid AAV-LK03 in both entry (DNA) and expression (cDNA) analyses in vivo using primary human hepatocytes (see FIG. 2B).

### Example 9. Identification of Additional Capsid Residues Important for In Vivo Transduction

Various AAV2 variant capsid polypeptides having a single amino acid substitution compared to the AAV2 set forth in SEQ ID NO:1 were vectorised: AAV2_C482S, AAV2_N496D (CMRI-19), AAV2_T503A, AAV2_K532E (CMRI-29), AAV2_N582S (CMRI_21), and AAV2_N596D. Each of these mutations had been enriched during in vivo screens of AAV2-based libraries, suggesting the modifications may have some importance. These were then tested for in vivo transduction of liver, along with, AAV2 containing all six mutations (AAV2.6 M). AAV2 having all mutations present in AAV2-NP59 (AAV2.V59) and AAV2 were included as controls. To enable a more robust analysis of data generated, two indexes were defined, the Entry Index (EI) and the Expression Index (EXI). EI corresponds to the quotient of capsid-specific NGS reads at the DNA level and the mapped NGS reads in the original vector mix, and defines the relative ability of a given capsid to physically transduce the targeted cells. EXI corresponds to the barcode-specific ratio of cDNA mapped reads to DNA mapped reads and offers a relative picture on the functional transduction of each capsid variant.

As shown in FIGS. 6A and B (and FIG. 4 of Australian provisional patent application no. 2019902374), improved in vivo physical and/or functional transduction was observed with most variants compared to AAV2. Three substitutions in particular (N496D, K532E and N582S) were sufficient to substantially increase both the entry and expression of AAV2 in human hepatocytes. The in vivo performance of AAV2_N496D (CMRI-19) and AAV2_N582S (CMRI21) were further confirmed using immunohistochemistry (FIG. 6C).

The improved in vivo transduction of the AAV2_K532E (CMRI_29) and AAV2_N496D (CMRI19) variants compared to prototypic AAV2 correlated with reduced heparin binding compared to AAV2, as shown in Table 4A. In addition to AAV2_K532E, AAV2_N496D and AAV2_T503A+N596D (CMRI_30), the heparin binding ability of other variants, including AAV2_R588I (CMRI_18), AAV2_R588T (CMRI_27) and AAV2_R487Q (CMRI_28), was assessed. These are shown here to exhibit significantly reduced binding compared to prototypic AAV2. Notably, the reduced binding of AAV2_R588I (CMRI_18) also correlates with improved in vivo transduction (see FIG. 2). Table 46 provides the results of a further heparin binding assay, which included an assessment of AAV2, AAV2_T503A+N596D (CMRI_30), AAV2.V59, AAV2_V12 (which is AAV2.V59 containing A503T+D596N relative to AAV2.59), AAV2_K532E (CMRI_29), AAV2_N582S (CMRI21) and AAV2_N496D (CMRI19). Notably, the binding capacity of AAV2_V12, which contains the AAV2-like residues at positions 503 and 596, is similar to AAV2, providing further confirmation that T503A+N596D mutations present in AAV2_T503A+N596D (CMRI_30) and AAV2.V59 (or NP59) are responsible for the reduced heparin binding observed in these vectors.

TABLE 4A

| | | | Conductivity | [NaCl] at |
|---|---|---|---|---|
| | | | at elution | elution |
| | | | peak | peak |
| | % in flow | % in elution | maxima | maxima |
| Vector | through | peak fraction | (mS/cm) | (mM) |
| AAV2 | 0.65% | 39.77% | 41.72 | 454.34 |
| AAV2 repeat | 0.41% | 28.38% | 41.25 | 452.75 |
| AAV2_N496D | 0.09% | 30.69% | 23.93 | 243.55 |
| AAV2_K532E | TBD | TBD | 26.05 | 281.00 |
| AAV2_T503A + | TBD | TBD | 34.51 | 380.45 |
| N596D | | | | |
| AAV2_R588I | 0.62% | 32.39% | 8.12 | 58.25 |
| AAV2_R588T | | | | 98.09 |
| AAV2_R487Q | 1.12% | 16.48% | 18 | 175.97 |

TABLE 4B

| | Predominantly | [NaCl] at elution peak |
|---|---|---|
| Vector | detected in: | maxima (mM) |
| AAV2 | Elution fraction | 453 |
| AAV2_T503A + | Elution fraction | 371 |
| N596D | | |
| AAV2_V12 | Elution fraction | 463 |
| AAV2_V59 | Elution fraction | 368 |
| AAV2_N496D | Elution fraction | 200 |
| AAV2_N582S | Elution fraction | 362 |
| AAV2_K532E | Elution fraction | 282 |

From the above studies, it is proposed that modification of the amino acid residues at positions 482, 496, 503, 532, 582, and/or 596, such as to effect an alteration in the charge of the capsid (e.g. introduce a negative charge), can reduce capsid affinity for heparin HSPG and thus enhance in vivo transduction of hepatocytes. Other positions proposed as being involved in HSPG binding include position 484, 487 and 589. Given the known contribution of residues at positions 585 and 588 to HSPG binding, modification of these residues should also reduce affinity for heparin/HSPG (as demonstrated in Table 4, above). Table 5 provides exemplary amino acid substitutions at positions 482, 484, 487, 496, 503, 532, 582, 585, 588, 589 and 596 that are proposed to reduce binding of the AAV vector to heparin/HSPG, and thus enhance in vivo transduction of hepatocytes.

TABLE 5

| Amino acid | Prototypic | |
|---|---|---|
| position | AAV2 aa | Substitution aa |
| 482 | Cysteine (C) | Serine (S) or threonine (T) |
| 484 | Arginine (R) | Glutamine (Q) or asparagine (N) |
| 487 | Arginine (R) | Glutamine (Q) or asparagine (N) |
| 496 | Asparagine (N) | Aspartic acid (D) or glutamic acid (E) |
| 503 | Threonine (T) | Alanine (A), valine (V) or isoleucine (I) |
| 532 | Lysine (K) | Glutamic acid (E) or glutamine (Q) |
| 582 | Asparagine (N) | Serine (S), aspartic acid (D), tyrosine |
| | | (Y), or threonine (T) |
| 585 | Arginine (R) | Serine (S), threonine (T), glycine (G), |
| | | alanine (A) or glutamic acid (E) |
| 588 | Arginine (R) | Threonine (T), isoleucine (I) or alanine |
| | | (A) |
| 589 | Glutamine (Q) | Aspartic acid (D), glutamic acid (E) or |
| | | alanine (A) |
| 596 | Asparagine (N) | Aspartic acid (D) or glutamic acid (E) |

### Example 10. Generation of Permutational Capsid Library and Selection of Capsids

A new permutational capsid library was produced. Each capsid gene (cap) in the library was based on the AAV2 cap set forth in SEQ ID NO:54 (Genbank Accession No. NC_001401; encoding the prototypic AAV2 capsid polypeptide set forth in SEQ ID NO:1) but having mutations that result in one or more amino acid substitutions in the encoded capsid polypeptide at position 482, 496, 503, 532, 582, 585, 588 and/or 596, as shown below in Table 6. Table 3, above, sets forth each of the combinations of amino acid residues at positions 482, 496, 503, 532, 582, 585, 588 and 596 of the AAV capsid polypeptides in this permutational library.

TABLE 6

| Amino acid | Prototypic AAV2 | |
|---|---|---|
| position | amino acid | Substitution amino acid |
| 482 | Cysteine (C) | Serine (S) |
| 496 | Asparagine (N) | Aspartic acid (D) |
| 503 | Threonine (T) | Alanine (A) |
| 532 | Lysine (K) | Glutamic acid (E) or glutamine (Q) |
| 582 | Asparagine (N) | Serine (S) |
| 585 | Arginine (R) | Serine (S) |
| 588 | Arginine (R) | Threonine (T) |
| 596 | Asparagine (N) | Aspartic acid (D) |

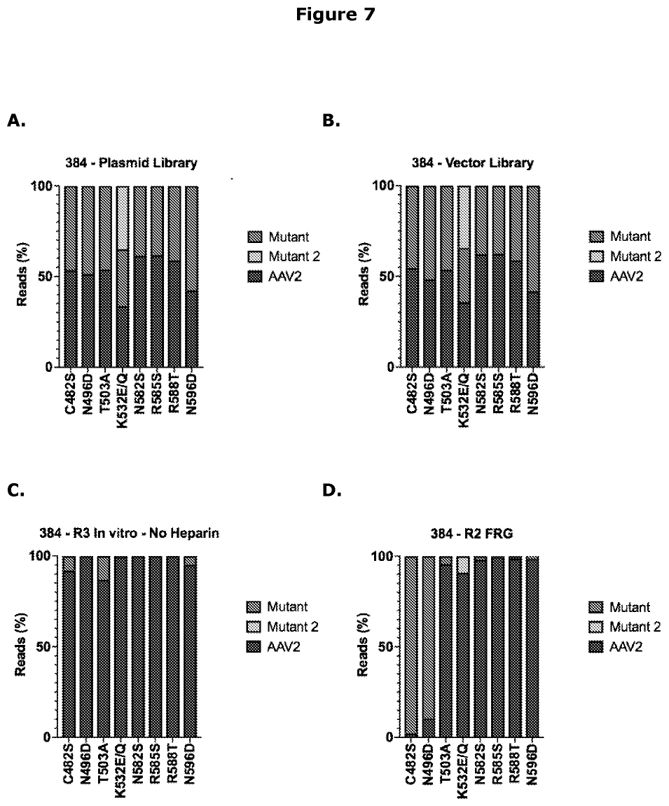

Selection of the library was then performed in vitro using HuH7 cells and in vivo using an hFRG mouse. NGS after 2 or 3 rounds of selection was performed to determine which, if any, amino acid substitution and capsid variants were enriched. As shown in FIG. 7, there was approximately 50% frequency of either the prototypic AAV2 amino acid residue or the “mutant” amino acid residue in the plasmid and vector libraries before selection for positions 482, 496, 503, 585, 582, 588 and 596, and approximately 33% frequency of the prototypic AAV2 amino acid residue, or “mutant” (i.e. E) or “mutant 2” (i.e. Q) residue at position 532, as expected. Following selection in vitro, the variants in the library shifted to a prototypic AAV2 sequence, i.e. the majority of the amino acid residues at each position represented the amino acid residues of the prototypic AAV2 capsid. In contrast, following selection in vivo, the modifications C482S and N496D were enriched. Tables 7 and 8 provide the capsid variants most enriched following in vitro or in vivo selection, respectively.

TABLE 7
Capsid variants enriched following in vitro selection

| Amino acid position (relative to SEQ ID NO: 1) | | | | | | | | Number of vectors |
|---|---|---|---|---|---|---|---|---|
| 482 | 496 | 503 | 532 | 582 | 585 | 588 | 596 | in round 2 |
| C | N | T | K | N | R | R | N | 321682 |
| C | N | A | K | N | R | R | N | 44964 |
| S | N | T | K | N | R | R | N | 20672 |
| C | N | T | K | N | R | R | D | 14539 |
| S | N | A | K | N | R | R | N | 8207 |
| S | N | T | K | N | R | R | D | 3696 |
| C | N | A | K | N | R | R | D | 1330 |
| C | N | T | Q | N | R | R | N | 902 |

TABLE 8
Capsid variants following in vivo selection in hFRG mouse

| Amino acid position (relative to SEQ ID NO: 1) | | | | | | | | Number of vectors |
|---|---|---|---|---|---|---|---|---|
| 482 | 496 | 503 | 532 | 582 | 585 | 588 | 596 | in round 3 |
| S | D | T | K | N | R | R | N | 370071 |
| S | N | T | Q | N | R | R | N | 16998 |
| S | D | T | Q | N | R | R | N | 15610 |
| S | N | T | K | N | R | R | N | 12222 |
| S | N | A | K | N | R | R | N | 5909 |
| S | D | T | K | S | R | R | N | 5004 |
| S | D | A | K | N | R | R | N | 3920 |
| S | D | T | K | N | R | R | D | 3558 |
| S | D | T | K | N | R | T | N | 3172 |
| S | D | T | K | N | G | R | N | 2963 |
| C | N | T | K | N | R | R | N | 2108 |
| C | D | T | K | N | R | R | N | 2008 |
| C | N | A | Q | N | R | R | N | 1933 |
| C | N | A | K | N | R | R | N | 1410 |
| C | N | A | Q | S | R | R | N | 1376 |
| S | N | A | Q | N | R | R | N | 1149 |
| S | N | A | K | N | R | T | N | 1003 |
| S | N | A | K | N | R | R | D | 917 |
| S | D | A | Q | N | R | R | N | 651 |
| S | D | T | Q | S | R | R | N | 638 |

Selection of the library was then performed in vivo using a juvenile long tailed macaque (Macaca fascicularis). The macaque was injected with 7.5×10¹¹ total vector genomes of the library, and two weeks post-injection, the animal was sacrificed. RNA was extracted from four independent sections of liver and cDNA was built with a Reverse primer cDNA_R (gaaacgaattaaacggtttattgattaacaagcaaTTACA—SEQ ID NO: 75, and a 434 BC region covering all the permutation positions was recovered via PCR with primers F_NGS (tctaggaactggcttcctggaccc—SEQ ID NO:76) and R_NGS (cagatgggcccctgaaggtacacatc—SEQ ID NO:77). The product was Illumina deep-sequenced with a 300-PE configuration. Table 9 shows the percentage of population reads for the 5 most enriched combination of mutations relative to the prototypic AAV2 set forth in SEQ ID NO:1 at positions 482, 496, 503, 585, 582, 588 and 596. These included the combinations of T503A+R585S+R588T+N596D; C482S+R585S; N496D+R588T+N596D; N496D+K532Q+R585S+R588T+N596D; and C482S+T503A+N596D.

TABLE 9

| | Percentage |
|---|---|
| | population |
| Mutation combination | reads |
| T503A + R585S + R588T + N596D | 14.85 |
| C482S + R585S | 12.36 |
| N496D + R588T + N596D | 14.34 |
| N496D + K532Q + R585S + R588T + N596D | 12.36 |
| C482S + T503A + N596D | 14.85 |

### Example 11. Inverse Correlation Between Heparin Binding and In Vivo Transduction in AAV8

A further study using AAV8 was performed to test the hypothesis in a reverse manner. The single mutation E533K and the double mutation Q588R/T591R have been described in the literature as being able to provide AAV8 capsids with stronger heparin binding (Woodard et al. J Virol 2016, 90(21):9878-9888; Vanderberghe et al. Nat Med 2006, 12(8):967-971). These positions correspond to positions 530, 585 and 588 of AAV2, respectively. Two AAV variant vectors were produced by introducing mutations into the prototypic AAV8 capsid polypeptide sequence set forth in SEQ ID NO:4 (Genbank Acc. No. NC_006261): AAV8_E533K and AAV8_RQNR (containing the Q588R and T591R mutations, respectively).

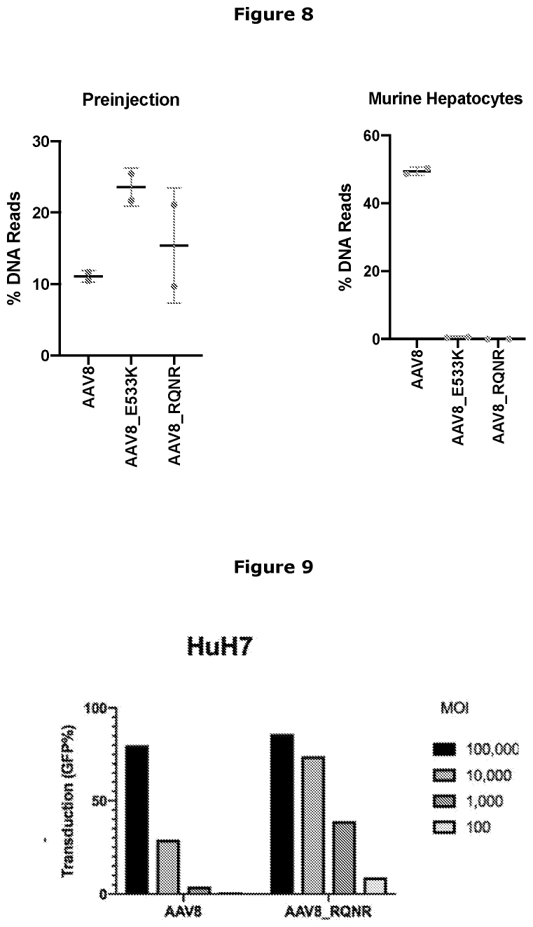

Both AAV8_E533K and AAV8_RQNR had increased binding to heparin compared to AAV8 when assessed using a heparin binding assay ([NaCl] at elution peak maxima (mM): AAV2=458 mM; AAV8=224 mM; AAV8_E533K=447 mM; AAV8_RQNR=598 mM). Based on the studies described above, it was hypothesized that the in vivo performance of these variants would be reduced compared to the prototypic AAV8 vector, while in vitro transduction of HuH-7 cells would be increased. This was essentially found to be the case. In vivo transduction of murine hepatocytes in hFRG mice was found to be negligible using AAV8. Conversely, an increase in in vitro HuH-7 transduction using AAV8_RQNR compared to AAV8 was observed (FIGS. 8-9).

Having shown that AAV8-E533K and AAV8-RQNR lost the ability to transduce murine hepatocytes compared to parental AAV8, the inventors investigated whether a single amino acid substitution could reduce the heparin affinity of AAV8-E533K and AAV8-RQNR and rescue their performance in vivo, as observed for AAV2. To do so, an N-to-D substitution at position 499, in silico predicted as the structural equivalent of AAV2-N496D, onto the heparin-binding AAV8 variants (referred as to AAV8-E533K-N499D and AAV8-RQNR-N499D). A heparin-binding assay confirmed the anticipated reduction in heparin affinity (where the concentration of NaCl at elution peak maxima was 650 mM for AAV8-RQNR; 369 mM for AAV8-E533K; 460 mM for AAV8-RQNR-N499D; and 271 mM for AAV8-E533K-N499D). The N499D mutation improved the ability of the AAV8 mutants to transduce murine hepatocytes, although, in contrast to AAV8, these variants appeared to transduce periportal hepatocytes with higher efficiency (data not shown).

### Example 12. Modification of AAV3B-Like Capsid to Alter Heparin Binding and In Vivo Transduction

It was hypothesised that AAV3B, or AA3B-like, capsid polypeptides could be modified in a similar manner to AAV2 so as to reduce heparin/HSPG binding and increase in vivo transduction efficiency of an AAV vector containing the modified capsid. Exemplary positions for modification include positions 476, 485, 488, 533, 594 and 598 relative to the prototypic AAV3B capsid polypeptide set forth in SEQ ID NO:56.

LK03 is an AAV3B-like capsid polypeptide with the sequence set forth in SEQ ID NO:70, having 8 amino acid substitutions compared to the prototypic AAV3B capsid polypeptide set forth in SEQ ID NO:56. LK03 is the prototypical AAV3B share the same HSPG binding region. Four variants of LK03 were produced: CMRI_31, containing a single N583S substitution compared to LK03 and having the sequence set forth in SEQ ID NO:71; CMRI_32, containing a single R488Q substitution compared to LK03 and having the sequence set forth in SEQ ID NO:72; CMRI_33, containing a single R594G substitution compared to LK03 and having the sequence set forth in SEQ ID NO:73; and CMRI34, containing a double R594E+D598H substitution compared to LK03 and having the sequence set forth in SEQ ID NO:74.

These variants were vectorised and their heparin binding affinity assessed as described above in Example 1. As shown in Table 10, the N583S substitution had no noticeable effect, with LK03 and CMRI_31 having similar heparin binding capacity. The R488Q substitution has a mild effect, with CMRI_32 having slightly decreased heparin binding capacity compared to LK03. The single R594G substitution and the double R594E+D598H substitution have much greater effects, with CMRI_33 and CMRI_34 having significantly decreased heparin binding capacity compared to LK03, where most of the vector was found in the flow through and only a small fraction bound the column. Also of note, the heparin binding capacity of LK03 is essentially the same as the prototypic AAV3B (see Table 11 below, where AAV3B has a [NaCl] at elution peak maxima of about 279 mM), as expected.

TABLE 10

| | | | Conductivity | |
|---|---|---|---|---|
| | | | at elution | |
| | | | peak | [NaCl] at |
| | % in flow | % in elution | maxima | elution peak |
| Vector | through | peak fraction | (mS/cm) | maxima |
| LK03 | 0% | 33% | 27.12 | 276.12 |
| CMRI_31 | 1% | Majority | 25.56 | 258.56 |
| CMRI_32 | 0% | Majority | 21.21 | 207.21 |
| CMRI_33 | Majority | Residual | 13.12 | 112.17 |
| CMRI_34 | Majority | Residual | 15.85 | 144.45 |

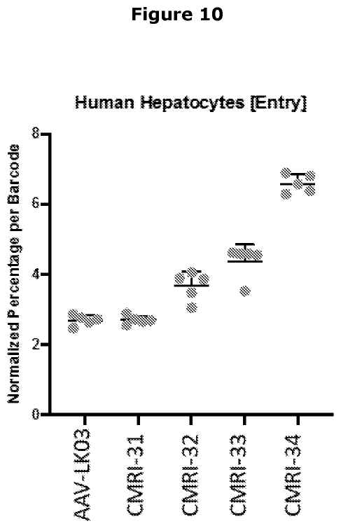

The in vivo transduction efficiency of the vectors was then assessed in an hFRG mouse as essentially described in Example 1. As shown in FIG. 10, heparin binding capacity generally inversely correlated with in vivo transduction efficiency, with CMRI_33 and CMRI34 having enhanced in vivo transduction efficiency compared to LK03. Indeed, CMRI_33 and CMRI34 made up 32.93% and 21.85%, respectively, of the vectors in the human hepatocytes, while LK03, CMRI_31 and CMRI_32 made up 13.43%, 13.48% and 18.32%, respectively.

### Example 13. In Vivo and In Vitro Performance and Heparin Binding of AAV Vectors Derived from AAV Liver Isolates

CMRI_01-CMRI_07, described above, were vectorized for in vivo functional analysis using hFRG mice.

Barcoded AAV transgenes (ITR-Liver Specific Promoter—eGFP—Barcode—WPRE—pA—ITR) were packaged into CMRI_01-CMRI_07, AAV2, AAV8. This allowed co-injection of the combined vectors into the same hFRG mouse for comparison of function. One week after injection of the AAV vectors (5×10¹⁰ vg per capsid; total of 4.5×10¹¹ vg); delivered intravenously into the lateral tail vein), the chimeric liver from the mouse was perfused and human and murine hepatocytes were single cell sorted. DNA was recovered from the human population of hepatocytes and Next Generation Sequencing (NGS) of the barcoded transgene was performed. This allowed comparison of the physical transduction of the novel capsids. The novel capsids were ˜11 to ˜82 times better than AAV2 at transducing human hepatocytes in vivo (data not shown).

A similar study was then performed, and the DNA specific for the vector barcodes in both human and mouse hepatocytes was quantified. It was observed that CMRI_01-CMRI_07 preferentially transduce human cells. CMRI_01 and CMRI_02 were then packaged with the canonical LSP-GFP vector described above and injected intravenously (2×10¹¹ vg/mouse) in one hFRG mouse each. Liver was collected two weeks after injection and analysed via immunohistochemistry. It was observed that the CMRI_01 and CMRI_02 vectors also preferentially transduced human cells (data not shown).

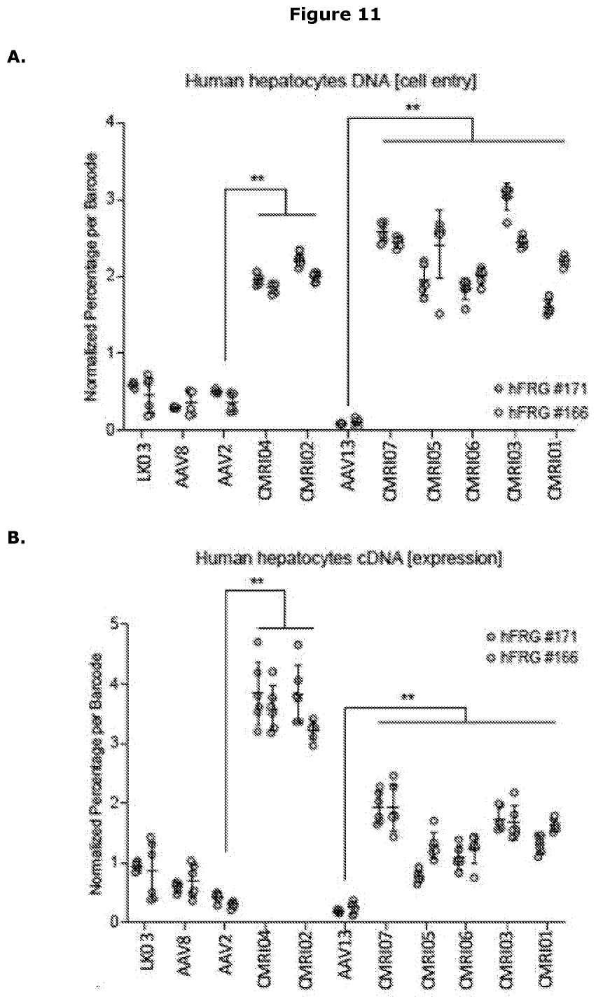

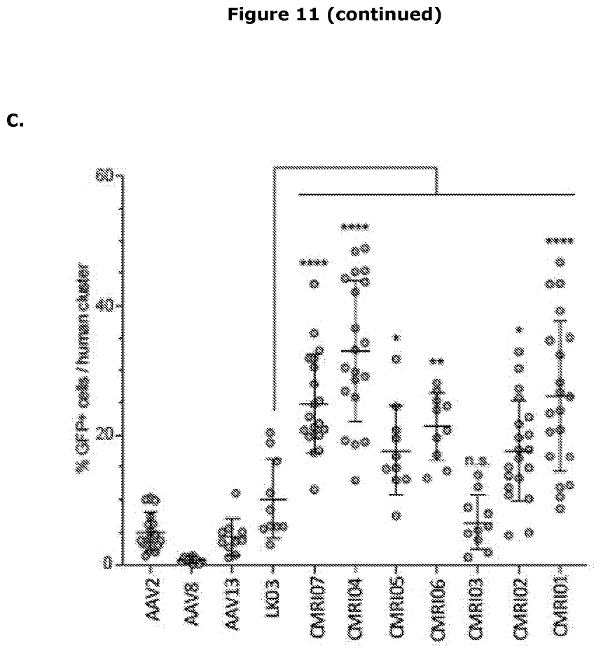

The above in vivo studies were essentially repeated, with reference serotypes AAV2 and AAV13, as well as AAV8 and AAV-LK03 as positive controls for functional transduction of murine and human hepatocytes, respectively. Two hFRG mice were injected intravenously with the total of 5.5×10¹¹ vg containing an equimolar mix of the individual variants (5×10¹⁰ vg of each capsid). Next generation sequence (NGS) analysis of the barcoded transgene was performed at the DNA (cell entry, physical transduction) and the RNA/cDNA (transgene expression, functional transduction) levels in mouse liver cells and human hepatocytes. Interestingly, all the liver-derived variants were more efficient at transduction of primary human hepatocytes than the respective closest reference serotype (AAV2 or AAV13), at both DNA and RNA levels (FIGS. 11A and B). As expected, based on the known ability of AAV8 to functionally transduce murine hepatocytes with high efficiency, the majority of barcodes recovered from murine hepatocytes at the cDNA level mapped to this capsid (data not shown). Consistent with the previous study, it was observed that the new variants were not efficient and transducing (either physically or functionally) mouse hepatocytes. Immunohistochemical analysis of variants in individual hFRG mice showed that six out of the seven novel liver-isolated variants were significantly more efficient at functional transduction of primary human hepatocytes than AAV-LK03, the positive control (FIG. 11C).

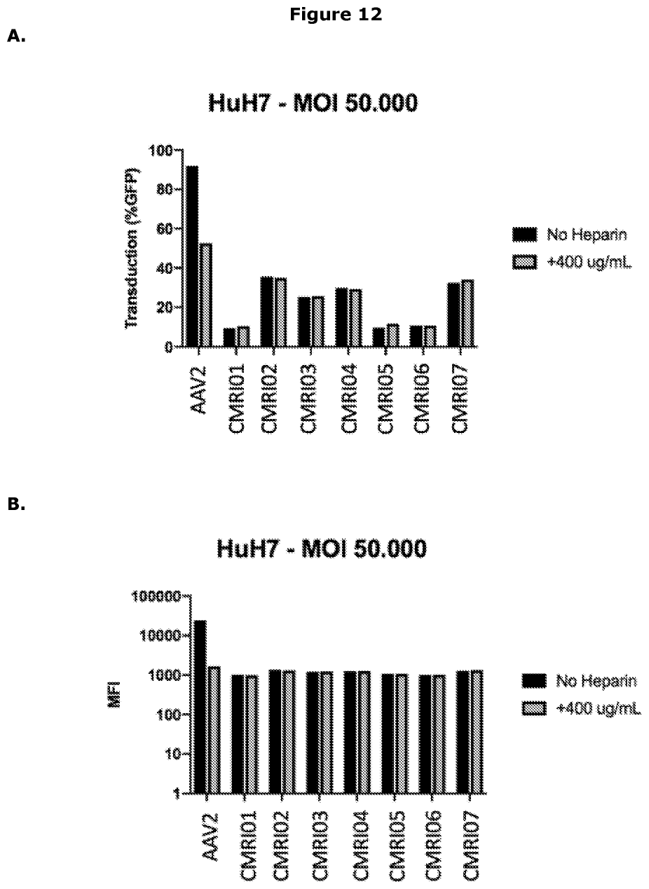

The in vitro performance of the vectors was tested in HuH-7 cells. All of the vectors appeared to have significantly lower transduction of cells in vitro compared to AAV2 in vitro, both in the number of cells transduced at MOI 50,000 (=5×10⁴) (FIG. 12). Notably, when soluble heparin (an HSPG analogue) was added to the media, AAV2 transduction was adversely affected, while transduction by CMRI_01-CMRI_07 vectors was not.

The heparin binding capability of the vectors was then assessed using the heparin binding assay. The heparin binding capability of the prototypic AAV2, AAV3B and AAV13 vectors were also assessed. As shown in Table 10, each of the CMRI_01-CMRI_07 vectors had significantly lower heparin binding than the prototypic AAV2 vector. Indeed, most of each of the CMRI_01-CMRI_07 vectors was detected in the flow through and not in the elution fraction, and the values for “[NaCl] at elution peak maxima” represent just a minor fraction of the vectors that bound to the column.

TABLE 11

| | | | Conductivity | |
|---|---|---|---|---|
| | | | at elution | |
| | | | peak | [NaCl] at |
| | % in flow | % in elution | maxima | elution peak |
| Vector | through | peak fraction | (mS/cm) | maxima |
| CMRI_01 | 21.58% | 1.31% | 13.51 | 122.86 |
| CMRI_02 | 68.89% | 5.80% | 16.98 | 161.74 |
| CMRI_03 | 68.76% | 5.28% | 15.89 | 151.52 |
| CMRI_04 | 63.48% | 3.24% | 15.18 | 141.44 |
| CMRI_05 | 42.37% | 7.51% | 15.59 | 147.11 |
| CMRI_06 | 75.18% | 3.22% | 23.65 | 241.11 |
| CMRI_07 | 59.97% | 2.22% | 14 | 127.48 |
| AAV2 | 0.65% | 39.77% | 41.72 | 454.34 |
| AAV2 repeat | 0.41% | 28.38% | 41.25 | 452.75 |
| AAV13 | 5.38% | 75.45% | 31.11 | 331.89 |
| AAV3B | 0.64% | 14/05% | 26.89 | 278.69 |

Comparison of the two AAV2-like sequences, CMRI_02 and CMRI_04, revealed that they lacked two critical arginine residues associated with AAV2 HSPG binding (R585 and R588), which is in keeping with most previously published Glade B variants detected in human tissues. Similarly, all AAV13-like variants lacked the structural equivalent key lysine 528 residue required for HSPG binding.

From the above, it is clear that human liver isolates interacted weakly, if at all, with heparin/HSPG (as demonstrated by the in vitro competition assay using soluble heparin as an analogue of HSPG, and the heparin binding assay). Conversely, prototypical AAV2 (which does have R585 and R588) did bind heparin and did transduce HuH-7 cells, transduction that was substantially inhibited by heparin.

### Example 14. In Vitro Adaptation of Wild-Type CMRI_01

As noted above, CMRI_01 appears to be an AAV13/AAV2 hybrid. Given the poor in vitro transduction performance of this isolate on HuH-7 cells, CMRI_01 was selected for studies to recapitulate the possible tissue culture adaptation of AAV13. To this end, a replication-competent (RC) version of the AAV13-like CMRI_01 variant (ITR2-rep2-CMRI_01-ITR2), referred to as RC-AAV-CMRI_01, was subjected to an in vitro adaptation protocol on HuH-7 cells in the presence of human Adenovirus 5 (hAd5). After four rounds of tissue culture adaptation, 98.57 of the isolated capsids harboured a glutamate to lysine change at position 530 (E530K) and 1.14% of clones had acquired an arginine at position 593 (G593R). Both these amino acids have positively charged side-chains and have been shown to facilitate heparin binding at corresponding structural regions in AAV13 and AAV3B, respectively. In depth analysis of the capsid gene region containing both modified residues using high-throughput NGS demonstrated that the original AAV-CMRI_01 was displaced rapidly after initial appearance of E530K and G593R in round 1 and 2, respectively. The same phenomenon was observed upon hAd5 supported iterative replication of a RC version of CMRI_04 in HuH-7 cells. The initial single variant population was rapidly dominated by a variant that acquired two point mutations, that resulted in removal of acidic amino acids at positions 469 (D469N) and 555 (E555K) (data not shown).

To test whether the observed changes in the AAV-CMRI_01 capsid sequence affected transduction characteristics, the two capsid variants were used to package the AAV-LSP-eGFP-WPRE-BGHpA cassette and were evaluated in vitro on HuH-7 cells. FACS analysis revealed that culture-adapted (ca)AAV-CMRI_01 E530K and caAAV-CMRI_01 G593R functionally transduced HuH-7 cells significantly more efficiently than CMRI_01. Furthermore, co-incubation with soluble heparin reduced the observed improvement in transduction to the level observed for wild-type AAV-CMRI_01 (data not shown; refer to FIG. 14 of Australian provisional patent application no. 2019903974). Moreover, in contrast to the wtAAV-CMRI_01, the culture-adapted variants (E530K and G593R) were shown to bind to the HiTrap Heparin Column (Concentration of NaCl at elution peak maxima was 320.89 mM for AAV-CMRI_01 E530K and 341.94 mM for AAV-CMRI_01 G593R, compared to 122.86 for CMRI_01, as shown above). This was anticipated as structural analysis revealed that the E530K change was adjacent to residues R487, K527, and K532, which are part of AAV13 HSPG binding region, and created a basic patch on the capsid surface in close proximity to R484 at base of the 3-fold protrusions (data not shown). An identical phenotype was observed for caAAV-CMRI_04 D469N+E555K, which in contrast to the natural serotype was found to bind to the HiTrap Heparin column and to transduce HuH-7 with significantly higher efficiency. These results suggest that besides AAV13, AAV3B may also represent a culture-adapted, HSPG-binding variant of natural AAVs.

The inventors next investigated whether change in HSPG-binding properties would have a detrimental effect in vivo. For that, six barcoded reporter constructs were packaged into reference serotypes AAV2, AAV8 and AAV13, as well as CMRI_01 and its culture-adapted version (caAAV-CMRI_01 E530K). Two hFRG mice were injected intravenously with the total of 2.5×10¹¹ vector genomes (vg) containing an equimolar mix of the individual variants (5×10¹⁰ vg of each capsid). Interestingly, the single amino acid substitution at the corresponding structural region in AAV13 (E530K) sufficed to significantly attenuate the in vivo performance of the otherwise highly human hepatotropic CMRI_01 to AAV13-like levels (data not shown).

### DISCUSSION

### Novel AAV Capsids

Multiple novel AAV capsid variants were produced and/or identified by a variety of methods, and the majority of these exhibited significantly improved in vivo transduction (physical and/or functional) of human hepatocytes compared to the prototypic AAV2.

### Correlation Between HSPG Binding and In Vivo Transduction of Human Hepatocytes

Improved human hepatotropism of AAV vectors is required to bring a higher proportion of liver diseases that are theoretically amenable to gene therapy within the technological reach of AAV-mediated gene transfer. The considerable recent advances in the development of functionally superior capsid variants, however, have not been paralleled by equal progress in elucidating the mechanisms that underpin these advances. In the above studies, the inventors used a sequence comparison between the AAV2 capsid and the bioengineered AAV-NP59 variant to understand the capsid-based determinants of in vivo human hepatotropism. These studies linked the superior in vivo performance of AAV-NP59 with reduced binding affinity to heparin, the experimental surrogate for HSPG. It was shown that in the context of AAV-NP59 this effect is driven solely by two amino acid substitutions, T503A and N596D. The synergistic effect of the combined amino acid substitutions on human liver transduction has gone unrecognised until the present disclosure. This insight was facilitated by the use of hFRG mice, which can show human hepatotropism in vivo, rather than in vitro models or in vivo mouse models.

Thus, based on the result described above, a functional model is proposed, in which upon reduction of the affinity of AAV2 towards HSPG, i) vector sequestration on extracellular matrixes is reduced, ii) the concentration of free vector increases, iii) the biodistribution of the vector increases, leading to iv) increased transduction of human hepatocytes in an HSPG-independent process. The final parameter of the model, which requires further investigation and validation, relates to the higher normalized expression (expression index) observed for the HSPG de-targeted variants compared to HSPG-binding counterparts. The data strongly suggest that, besides biodistribution and cellular entry, strong HSPG binding through direct or indirect mechanisms, such as those affecting intracellular trafficking, negatively affects post-entry steps leading to transgene expression, further lowering the overall functional efficiency of HSPG-binding vectors.

The studies above underscore the importance of preclinical testing in biologically-predictive model systems, since the in vitro results using human hepatocyte-derived cell lines would be misleading in terms of clinical performance. It is logical to hypothesize that due to the lack of HSPG-rich extracellular matrix affecting capsid biodistribution, increased HSPG binding is beneficial for the in vitro performance of AAV2. An interesting question that arises is why the prototypical AAV2 would present such a high affinity to HSPG if this property was theoretically detrimental for in vivo spreading. As discussed further below, the ability of AAV2 to bind HSPG could constitute a tissue culture adaptation acquired during serial passaging in the presence of Adenovirus in vitro. Importantly, this artificial property could also directly contribute to the low yields in the purification process typically observed for AAV2, as it has been previously proposed that strong binding to HSPG could lead to the loss of vector particles in the cell debris. The fact that all the AAV2 variants with decreased HSPG binding yielded a higher number of packaged particles per cell than AAV2 supports this hypothesis. The data further suggest that vectors with decreased HSPG binding will enhance translational studies through improved function and enhanced manufacturing, potentially facilitating therapeutic benefits at a lower vector dose (improved safety) and lower cost per patient (improved healthcare impact).

Interestingly, another previously described functional AAV variant, AAV-NP40, which, similarly to AAV-NP59, is also closely related to AAV2 (12 amino acid differ between the variants), does not harbor the key substitutions (T503A and N596D) responsible for improved hepatotropism of AAV-NP59. Guided by the increased understanding of the relationship between HSPG binding and vector hepatotropism, the inventors investigated whether AAV-NP40 harbored any substantial changes within the heparin binding domains situated at the capsid three-fold protrusions. Pairwise alignment of this variant with AAV2 revealed that AAV-NP40 carries a lysine for glutamate change on K532E, one of the five key amino acids that has historically been associated with HSPG interaction. Another previously described variant, AAV-NP84, carries an additional potentially HSPG de-targeting substitution at the key arginine 585 (R585G). Selection studies described here enriched for these mutations and also identified three additional enriched substitutions (C482S, N496D, and N582S), two of which were shown to substantially improve human hepatotropism of AAV2.

The studies described above show for the first time the correlation between HSPG/heparin binding of AAV capsids/vectors (e.g. AAV2 and AAV13 capsids/vectors) and in vivo transduction of human hepatocytes. The studies demonstrate that modification of various amino acid residues in AAV2 or AAV13 capsids so as to reduce HSPG/heparin binding can enhance in vivo transduction of human hepatocytes. In some instances, the modifications effect an alteration in the charge of the capsid (e.g. introduce a negative charge) so as to reduce capsid affinity for heparin/HSPG and thus enhance in vivo transduction of hepatocytes. As proposed herein, exemplary modifications include one or more of those in Table 5 or Table 6.

### Likely Culture Adaptation of Prototypic AAV2

Using a clinically predictive xenograft model of human liver, the studies reported here provide direct evidence that prototypical AAV2, from which the AAV vector system was first derived, is in all probability not the original primary isolate, but rather an HSPG-binding variant arising in culture. The results provide a direct link between increased HSPG binding and resultant attenuation of tropism for primary human hepatocytes in vivo and suggest a mechanism explaining the low efficacy of the AAV2 capsid when first deployed in the human liver in a clinical trial targeting haemophilia B. Finally, the studies in a clinically predictive xenograft model provide direct evidence that novel natural AAV variants isolated from human liver can transduce primary human hepatocytes with clinically-relevant efficiencies, making them ideal candidates for liver-targeted gene therapy applications.

Over sixteen years elapsed between the isolation of the prototypical AAV2, its sequencing and genome annotation and subsequent vectorisation, during which the virus was propagated in vitro on KB cells in the presence of Ad12 and on HeLa cells in the presence of Ad2. The results above demonstrate unequivocally that during in vitro propagation replication competent AAVs acquire random mutations, and that variants with DNA mutations leading to specific capsid residue changes that markedly enhance interaction with HSPG quickly expand on the population. Importantly, while advantageous in tissue culture, clinical and preclinical data in chimeric mouse human livers indicate that this property is detrimental to the human liver-directed transduction in vivo.

The observation that the novel human liver isolates demonstrate strikingly opposite performance from prototypical AAV2 in vitro and in vivo supports the hypothesis that the ability to bind HSPG with high affinity might be an artificial viral property and therefore not a requirement for natural infection in humans. However, the immensely important consequences of the hypothesised in vitro adaptation for the liver-directed clinical utility of vectors derived from prototypical AAV2 have gone unrecognized. This has negatively affected clinical development of AAV vectors and has significant historical significance for the field of liver-targeted gene therapy where the prototypical culture-isolated AAV2 variant showed low efficiency in clinical trial targeting the human liver. Based on the data presented herein this unanticipated and surprising clinical result can likely be attributed to low tissue penetration due to vector sequestration on the HSPG-rich extracellular matrices in liver and other tissues. The fact that replication-competent AAV2 rapidly lost heparin binding capacity in the in vivo re-adaptation experiments further supports the hypothesis that strong HSPG binding has a negative effect on human liver transduction. Importantly, while deficient HSPG binding has previously been shown to markedly reduce AAV transduction of the murine liver, here it is shown for the first time that lack or reduction of heparin binding, in the context of natural and bioengineered capsid AAV variants, dramatically improves functional in vivo transduction of primary human hepatocytes to levels superior to AAV-LK03, the first AAV capsid bioengineered for human liver tropism to reach the clinic and currently in a Phase III trial for haemophilia A.

The in vivo data clearly show that the relative level of transduction when comparing multiple barcoded vectors using NGS, changes depending if DNA or RNA (cDNA) is analysed. Interestingly, AAV13-like variants appear to physically transduce human hepatocytes with higher efficiencies than AAV2-like variants, but generally are less efficient when analysed at the RNA (cDNA) level. While this observation points toward differences in post cell entry trafficking between variants, further studies will be required to understand the underlying mechanisms. Nonetheless, despite those differences, analysis of multiple barcoded vectors in a single xenografted animal allows to overcome potential animal to animal differences, such as the different levels of engraftment with human cells, effect of human cluster size on transduction efficiency or variations in injection efficiency. Analysis of GFP reporter expression in hFRG mice injected with individual variants confirmed the overall superior transduction o natural variants when compared to AAV-LK03 in this model, positioning the new variants as promising tools for clinical applications and as parental capsids for the next generation of engineered variants.

In the absence of sequence data for the original 1966 AAV2 isolate, it may never be known whether the prototypical AAV2 adapted to the tissue culture environment via de novo mutations that occurred in vitro or expanded from a naturally occurring rare variant. However, in keeping with the majority of AAV2-like sequences derived from human liver, the novel highly functional primary human hepatocyte AAV isolates described herein lack arginine residues at capsid positions 585 and 588 and do not bind heparin, an analogue of HSPG. Thus, the data confirms that strong HSPG binding attenuates the in vivo tropism of AAV2-like capsids for human hepatocytes. Referring to the re-adaptation study, the fact that variants with acquired HSPG binding achieved population dominance in vitro, and, on the contrary, HSPG-detargeted variants achieved dominance in vivo, also strongly suggests a direct relationship between HSPG binding strength and viral performance. Nevertheless, other infection-determining factors not related to HSPG could also contribute to the observed shift in viral population.

The paradigm of AAV culture adaptation and concomitant gain of strong HSPG binding appears to extend to at least some other prototypical serotypes. As mentioned above, the five novel liver-derived AAV clade C capsid sequences, along with all previously published human tissue-derived clade C isolates, resembled AAV13 in terms of heparin-binding residues, with one notable exception: whereas the AAV13 capsid sequence is characterised by a K528 that is critical for AAV13 heparin binding, the equivalent amino acid in variants isolated directly from human and non-human primate liver and other tissues is a negatively charged E530. It is postulated that all the prototypical serotypes reported to bind to HSPG, such as AAV3A, AAV3B, AAV6 and AAV13, which like AAV2 were passaged in vitro in the presence of adenovirus before their initial sequencing, may represent culture-adapted variants of the original natural isolates.

Of significant importance to the future of liver-targeted gene therapy, the studies above are the first to show that AAV vectors derived directly from human liver, and which lack or show reduced affinity for HSPG as indicated by reduced affinity to heparin, transduce primary human hepatocytes in vivo in a xenograft model of human liver with clinically-relevant efficiency. Furthermore, based on functional evaluation of the natural variants it is hypothesised that primary tissues other than liver may also serve as a fertile source of highly functional AAV variants for clinical applications targeting different organs.

Capsid polypeptide sequences
CMRI 01 (SEQ ID NO: 2):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGSTTMATGSGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQTNSGTLQQSRLLFSQAGPTNMSLQAKNWLPGPCYRQQRLSKQANDNNN
SNFPWTAATKYHLNGRDSLVNPGPAMASHKDDEEKFFPMHGTLIFGKQGTNANDADLENVMITDEEEIRTT
NPVATEQYGTVSNNLQNSNTGPTTGTVNHQGALPGMVWQDRDVYLQGPIWAKIPHTDGHFHPSPLMGGFG
LKHPPPQIMIKNTPVPANPPTNFSSAKFASFITQYSTGQVSVEIEWELQKENSKRWNPEIQYTSNYNKSVNVD
FTVDTNGVYSEPRPIGTRYLTRNL
CMRI 02 (SEQ ID NO: 3):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRILEPLGLVEEPVKTAPGKK
RPVEHSPAEPDSSSGTGKSGQQPARKRLNFGQTGDADSVPDPQPLGQPPAAPSGLGTNTMASGSGAPMAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTMSRLQFSQAGASDIRDQSRNWLPGPCYRQQRVSKTAADNNN
SDYSWTGATKYHLNGRDSLVNPGPAMASHKDDEEKYFPQSGVLIFGKQDSGKTNVDIEKVMITDEEEIRTT
NPVATEQYGSVSTNLQSGNTQAATSDVNTQGVLPGMVWQDRDVYLQGPIWAKIPHTDGHFHPSPLMGGFG
LKHPPPQILIKNTPVPANPSTTFSAAKFASFITQYSTGQVSVEIEWELQKENSKRWNPEIQYTSNYNKSVNVD
FTVDTNGVYSEPRPIGTRYLTRNL
CMRI 03 (SEQ ID NO: 4):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPAEPDSSSGTGKAGQQPARKRLNFGQTGDADSVPDPQPLGEPPAAPTSLGSTTMASGGGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNRTQTASGTQQSRLLFSQAGPTSMSLQAKNWLPGPCYRQQRLSKQANDNNNS
NFPWTAATKYHLNGRDSLVNPGPAMASHKDDEEKFFPMHGTLIFGKEGTNANNAELENVMITDEEEIRTTNP
VATEQYGYVSNNLQNSNTAATTETVNHQGALPGMVWQDRDVYLQGPIWAKIPHTDGHFHPSPLMGGFGLK
HPPPQIMIKNTPVPANPPTNFSAAKFASFITQYSTGQVSVEIEWELQKENSKRWNPEIQYTSNYNKSVNVDFT
VDTNGVYSEPRPIGTRYLTRNL
CMRI 04 (SEQ ID NO: 5):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSKTNAPSGTTTMSRLQFSQAGASDIRDQSRNWLPGPCYRQQRVSKTAADNNN
SDYSWTGATKYHLNGRDSLVNPGTAMASHKDDEEKYFPQSGVLIFGKQDSGKTNVDIEKVMITDEEEIRTT
NPVATEQYGSVSTNLQSGNTQAATSDVNTQGVLPGMVWQDRDVYLQGPIWAKIPHTDGHFHPSPLMGGFG
LKHPPPQILIKNTPVPANPSTTFSAAKFASFITQYSTGQVSVEIEWELQKENSKRWNPEIQYTSNYNKSVNVD
FTVDTNGVYSEPRPIGTRYLTRNL
CMRI 05 (SEQ ID NO: 6):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPAEPDSSSGTGKAGQQPAKKRLNFGQTGDSDSVPDPQPLGEPPAAPTSLGSTTMASGGGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQSASGTVQQSRLLFSQAGPTSMSLQAKNWLPGPCYRQQRLSKQANENNN
SNFPWTAATKYHLNGRDSLVNPGPPMASHKDDEEKFFPMHGTLIFGKEGTNANNAELENVMITDEEEIRTTN
PVATEQYGNVSNNLQNSNTAPSTGTVNHQGALPGMVWQDRDVYLQGPIWAKIPHTDGHFHPSPLMGGFGL
KHPPPQIMIKNTPVPANPPTNFSAAKFASFITQYSTGQVSVEIEWELQKENSKRWNPEIQYTSNYNKSVNVDF
TVDTNGVYSEPRPIGTRYLTRNL
CMRI 06 (SEQ ID NO: 7):
MAADGYLPDWLEDTLSEGIRQWWKLKPGPPPPKPAERHKDNSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPAKKRLNFGQTGDADSVPDPQPLGQPPAAPSGLGSTTMATGSGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQSNSGTLQQSRLLFSQAGPTSMSLQAKNWLPGPCYRQQRLSKQANDNNN
SNFPWTAATKYHLNGRDSLVNPGPAMASHKDDEEKFFPMHGTLIFGKQGTNANDADLDNVMITDEEEIRTT
NPVATEQYGYVSNNLQNSNTGPTTGTVNHQGALPGMVWQDRDVYLQGPIWAKIPHTDGHFHPSPLMGGFG
LKHPPPQIMIKNTPVPANPPTNFSSAKFASFITQYSTGQVSVEIEWELQKENSKRWNPEIQYTSNYNKSVNVD
FTVDTNGVYSEPRPIGTRYLTRNL
CMRI 07 (SEQ ID NO: 8):
MAADGYLPDWLEDTLSEGIRQWWKLKPGPPPPKPAERHKDDSRGLVLPGYKYLGPFNGLDKGEPVNEADAA
ALEHDKAYDRQLNSGDNPYLKYNHADAEFQERLKEDTSFGGNLGRAVFQAKKRVLEPLGLVEEPVKTAPGKK
RPVEHSPAEPDSSSGTGKAGQQPARKRLNFGQTGDADSVPDPQPLGEPPAAPTSLGSTTMASGGGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQNSSGTVQQSRLLFSQAGPTSMSLQAKNWLPGPCYRQQRLSKQASDNN
NSNFPWTAATKYHLNGRDSLVNPGPAMASHKDDEEKFFPMHGTLIFGKEGTTANNADLEHVMITDEEEIRTT
NPVATEQYGNVSNNLQNSNTGPTTENVNNQGALPGMVWQDRDVYLQGPIWAKIPHTDGHFHPSPLMGGF
GLKHPPPQIMIKNTPVPANPPTNFSAAKFASFITQYSTGQVSVEIEWELQKENSKRWNPEIQYTSNYNKSVNV
DFTVDTNGVYSEPRPIGTRYLTRNL
CMRI 08 (SEQ ID NO: 9):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPAEPDSSSGTGKAGQQPARKRLNFGQTGDADSVPDPQPLGEPPAAPTSVGSTTMASGGGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQNSSGTVQQSRLLFSQAGPTSMSLQAKNWLPGPCYRQQRLSKQASDNN
NSNFPWTAATKYHLNGRDSLVNPGPAMASHKDDEEKFFPMHGTLIFGKEGTTANNADLEHVMITDEEEIRTT
NPVATEQYGNVSNNLQNSNTGPTTENVNNQGALPGMVWQDRDVYLQGPIWAKIPHTDGHFHPSPLMGGF
GLKHPPPQIMIKNTPVPANPPTNFSAAKFASFITQYSTGQVSVEIEWELQKENSKRWNPEIQYTSNYNKSVNV
DFTVDTNGVYSEPRPIGTRYLTRNL
CMRI 09 (SEQ ID NO: 10):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKKDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGSTTMATGSGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQSNSGTLQQSRLLFSQAGPTSMSLQAKNWLPGPCYRQQRLSKQANDNNN
SNFPWTAATKYHLNGRDSLVNPGPAMASHKDDEEKFFPMHGTLIFGKQGTNANDADLDNVMITDEEEIRTT
NPVATEQYGYVSNNLQNSNTGPTTGTVNHQGALPGMVWQDRDVYLQGPIWAKIPHTDGHFHPSPLMGGFG
LKHPPPQIMIKNTPVPANPPTNFSSAKFASFITQYSTGQVSVEIEWELQKENSKRWNPEIQYTSNYNKSVNVD
FTVDTNGVYSEPRPIGTRYLTRNL
CMRI 10 (SEQ ID NO: 11):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPAEPDSSSGTGKAGQQPAKKRLNFGQTGDSDSVPDPQPLGEPPAAPTSLGSTTMASGGGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQSASGTVQQSRLLFSQAGPTSMSLQAKNWLPGPCYRQQRLSKQANENNN
SNFPWTAATKYHLNGRDSLVNPGPPMASHKDDEEKFFPMHGTLIFGKEGTNANNAELENVMITDEEEIRTTN
PVATEQYGNVSNNLQNSNTAPSTGTVNHQGALPGMVWQDRDVYLQGPIWAKIPHTDGHFHPSPLMGGFGL
KHPPPQIMIKNTPVPANPPTNFSAAKFASFITQYSTGQVSVEIEWELQKENSKRWNPEIQYTSNYNKSVNVDF
TVDTNGVYSEPRPIGTRYLTRNL
CMRI 11 (SEQ ID NO: 12):
MAADGYLPDWLEDTLSEGIRQWWKLKPGPPPPKPAERHKDDSRGLVLPGYKYLGPFNGLDKGEPVNAADAA
ALEHDKAYDQQLKAGDNPYLRYNHADAEFQERLQEDTSFGGNLGRAVFQAKKRVLEPLGLVEEGAKTAPGK
KRPVEPSPQRSPDSSTGIGKKGQQPAKKRLNFGQTGDSESVPDPQPLGEPPAAPSGLGTNTMATGSGAPMA
DNNEGADGVGNSSGNWHCDSTWMGDRVITTSTRTWALPTYNNHLYKQISSASTGASNDNHYFGYSTPWG
YFDFNRFHCHFSPRDWQRLINNNWGFRPKRLNFKLFNIQVKEVTTNDGVTTIANNLTSTVQVFSDSEYQLPY
VLGSAHQGCLPPFPADVFMIPQYGYLTLNNGSQAVGRSSFYCLEYFPSQMLRTGNNFQFSYTFEDVPFHSSY
AHSQSLDRLMNPLIDQYLYYLSRTNTPSGTTTQSRLQFSQAGASDIRDQSRNWLPGPCYRQQRVSKTSADN
NNSEYSWTGATKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHPSPLMGG
FGLKHPPPQILIKNTPVPANPSTTFSPAKFASFITQYSTGQVSVEIEWELQKENSKRWNPEIQYTSNYNKSVN
VDFTVDTNGVYSEPRPIGTRYLTRNL
CMRI 12 (SEQ ID NO: 13):
MAADGYLPDWLEDNLSEGIRQWWALKPGAPKPKANQQKQDDGRGLVLPGYKYLGPFNGLDKGEPVNAAD
AAALEHDKAYDQQLKAGDNPYLRYNHADAEFQERLQEDTSFGGNLGRAVFQAKKRVLEPLGLVEEGAKTAP
GKKRPVEPSPQRSPDSSTGIGKKGQQPAKKRLNFGQTGDSESVPDPQPLGEPPAAPSGLGTNTMATGSGAP
MADNNEGADGVGNSSGNWHCDSTWMGDRVITTSTRTWALPTYNNHLYKQISSASTGASNDNHYFGYSTP
WGYFDFNRFHCHFSPRDWQRLINNNWGFRPKRLNFKLFNIQVKEVTTNDGVTTIANNLTSTVQVFSDSEYQ
LPYVLGSAHQGCLPPFPADVFMIPQYGYLTLNNGSQAVGRSSFYCLEYFPSQMLRTGNNFQFSYTFEDVPFHS
SYAHSQSLDRLMNPLIDQYLYYLSRTNTPSGTTTQSRLQFSQAGASDIRDQSRNWLPGPCYRQQRVSKTSA
DNNNSEYSWTGATKYHLNGRDSLVNPGPAMASHKDDEEKFFPMHGNLIFGKEGTTASNAELDNVMITDEEE
IRTTNPVATEQYGTVANNLQSSNTAPTTRTVNDQGALPGMVWQDRDVYLQGPIWAKIPHTDGHFHPSPLMG
GFGLKHPPPQILIKNTPVPANPSTTFSPAKFASFITQYSTGQVSVEIEWELQKENSKRWNPEIQYTSNYNKSV
NVDFTVDTNGVYSEPRPIGTRYLTRNL
CMRI 13 (SEQ ID NO: 14):
MAADGYLPDWLEDTLSEGIRQWWKLKPGPPPPKPAERHKDDSRGLVLPGYKYLGPFNGLDKGEPVNAADAA
ALEHDKAYDQQLKAGDNPYLRYNHADAEFQERLQEDTSFGGNLGRAVFQAKKRVLEPLGLVEEGAKTAPGK
KRPVEPSPQRSPDSSTGIGKKGQQPAKKRLNFGQTGDSESVPDPQPLGEPPAAPSGLGTNTMATGSGAPMA
DNNEGADGVGNSSGNWHCDSTWMGDRVITTSTRTWALPTYNNHLYKQISSASTGASNDNHYFGYSTPWG
YFDFNRFHCHFSPRDWQRLINNNWGFRPKRLNFKLFNIQVKEVTTNDGVTTIANNLTSTVQVFSDSEYQLPY
VLGSAHQGCLPPFPADVFMIPQYGYLTLNNGSQAVGRSSFYCLEYFPSQMLRTGNNFQFSYTFEDVPFHSSY
AHSQSLDRLMNPLIDQYLYYLSRTNTPSGTTTQSRLQFSQAGASDIRDQSRNWLPGPCYRQQRVSKTSADN
NNSEYSWTGATKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHPSPLMGG
FGLKHPPPQILIKNTPVPANPSTTFSPAKFASFITQYSTGQVSVEIEWELQKENSKRWNPEIQYTSNYNKSVN
VDFTVDTNCVYSEPRPIGTRYLTRNL
CMRI 14 (SEQ ID NO: 15):
MAADGYLPDWLEDTLSEGIRQWWKLKPGAPKPKANQQKQDDGRGLVLPGYKYLGPFNGLDKGEPVNAADA
AALEHDKAYDQQLKAGDNPYLRYNHADAEFQERLQEDTSFGGNLGRAVFQAKKRVLEPLGLVEEGAKTAPG
KKRPVEPSPQRSPDSSSGIGKTGQQPAKKRLNFGQTGDSESVPEPQPLGEPPATPAAVGPTTMASGGGAPM
ADNNEGADGVGNASGNWHCDSQWLGDRVITTSTRTWALPTYNNHLYKQISSASTGASNDNHYFGYSTPW
GYFDFNRFHCHFSPRDWQRLINNNWGFRPKRLNFKLFNIQVKEVTTNDGVTTIANNLTSTVQVFSDSEYQLP
YVLGSAHQGCLPPFPADVFMIPQYGYLTLNNGSQAVGRSSFYCLEYFPSQMLRTGNNFQFSYTFEDVPFHSSY
AHSQSLDRLMNPLIDQYLYYLSRTNTPSGTTTQSRLQFSQAGASDIRDQSRNWLPGPCYRQQRVSKTSADN
NNSEYSWTGATKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHPSPLMGG
FGLKHPPPQILIKNTPVPANPSTTFSPAKFASFITQYSTGQVSVEIEWELQKENSKRWNPEIQYTSNYNKSVN
VDFTVDTNCVYSEPRPIGTRYLTRNL
CMRI 15 (SEQ ID NO: 16):
MAADGYLPDWLEDNLSEGIREWWDLKPGAPKPKANQQKQDDGRGLVLPGYKYLGPFNGLDKGEPVNAADA
AALEHDKAYDQQLKAGDNPYLRYNHADAEFQERLQEDTSFGGNLGRAVFQAKKRVLEPLGLVEEAAKTAPG
KKRPVEPSPQRSPDSSSGIGKTGQQPAKKRLNFGQTGDSESVPDPQPLGEPPATPAAVGPTTMASGGGAPM
ADNNEGADGVGNSSGNWHCDSQWMGDRVITTSTRTWALPTYNNHLYKQISSQSGASNDNHYFGYSTPW
GYFDFNRFHCHFSPRDWQRLINNNWGFRPKRLSFKLFNIQVKEVTQNEGTKTIANNLTSTIQVFTDSEYQLPY
VLGSAHQGCLPPFPADVFMIPQYGYLTLNNGSQSVGRSSFYCLEYFPSQMLRTGNNFQFTYTFEDVPFHSSYA
HSQSLDRLMNPLIDQYLYYLSRTNTPSGTTTQSRLQFSQAGASDIRDQSRNWLPGPCYRQQRVSKTSADNN
NSEYSWTGATKYHLNGRDSLVNPGPAMASHKDDEEKFFPMHGNLIFGKEGTTASNAELDNVMITDEEEIRTT
NPVATEQYGTVANNLQSSNTAPTTRTVNDQGALPGMVWQNRDVYLQGPIWAKIPHTDGNFHPSPLMGGFG
LKHPPPQILIKNTPVPADPPTTFSQAKLASFITQYSTGQVSVEIEWELQKENSKRWNPEIQYTSNYYKSTSVDF
AVNTEGVYSEPRPIGTRYLTRPL
CMRI 16 (SEQ ID NO: 17):
MAADGYLPDWLEDTLSEGIRQWWKLKPGAPKPKANQQKQDDGRGLVLPGYKYLGPFNGLDKGEPVNAADA
AALENDKAYDQQLKAGDNPYLRYNHADAEFQERLQEDTSFGGNLGRAVFQAKKRVLEPLGLVEEGAKTAPG
KKRPVEPSPQRSPDSSTGIGKKGQQPAKKRLNFGQTGDSESVPDPQPLGEPPAAPSGLGTNTMATGSGAPM
ADNNEGADGVGNSSGNWHCDSTWMGDRVITTSTRTWALPTYNNHLYKQISSASTGASNDNHYFGYSTPW
GYFDFNRFHCHFSPRDWQRLINNNWGFRPKRLNFKLFNIQVKEVTTNDGVTTIANNLTSTVQVFSDSEYQLP
YVLGSAHQGCLPPFPADVFMIPQYGYLTLNNGSQAVGRSSFYCLEYFPSQMLRTGNNFQFSYTFEDVPFHSSY
AHSQSLDRLMNPLIDQYLYYLSRTNTPSGTTTQSRLQFSQAGASDIRDQSRNWLPGPCYRQQRVSKTSADN
NNSEYSWTGATKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHPSPLMGG
FGLKHPPPQILIKNTPVPANPSTTFSPAKFASFITQYSTGQVSVEIEWELQKENSKRWNPEIQYTSNYNKSVN
VDFTVDTNCVYSEPRPIGTRYLTRNL
CMRI 17 (SEQ ID NO: 18):
MAADGYLPDWLEDNLSEGIREWWDLKPGAPKPKANQQKQDDGRGLVLPGYKYLGPFNGLDKGEPVNEADA
AALEHDKAYDRQLDSGDNPYLKYNHADAEFQERLKEDTSFGGNLGRAVFQAKKRILEPLGLVEEAAKTAPGK
KRPVEPSPQRSPDSSSGIGKTGQQPAKKRLNFGQTGDSESVPDPQPLGEPPATPAAVGPTTMASGGGAPMA
DNNEGADGVGNSSGNWHCDSQWMGDRVITTSTRTWALPTYNNHLYKQISSASTGASNDNHYFGYSTPW
GYFDFNRFHCHFSPRDWQRLINNNWGFRPKRLSFKLFNIQVKEVTQNEGTKTIANNLTSTIQVFTDSEYQLPY
VLGSAHQGCLPPFPADVFMIPQYGYLTLNNGSQSVGRSSFYCLEYFPSQMLRTGNNFQFTYTFEDVPFHSSYA
HSQSLDRLMNPLIDQYLYYLSRTNTPSGTTTQSRLQFSQAGASDIRDQSRNWLPGPCYRQQRVSKTSADNN
NSEYSWTGATKYHLNGRDSLVNPGPAMASHKDDEEKFFPMHGNLIFGKEGTTASNAELDNVMITDEEEIRTT
NPVATEQYGTVANNLQSSNTAPTTRTVNDQGALPGMVWQNRDVYLQGPIWAKIPHTDGNFHPSPLMGGFG
LKHPPPQILIKNTPVPADPPTTFSQAKLASFITQYSTGQVSVEIEWELQKENSKRWNPEIQYTSNYYKSTSVDF
AVNTEGVYSEPRPIGTRYLTRPL
CMRI 18 (SEQ ID NO: 19):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYGYSTPWGYH
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTQSRLQFSQAGASDIRDQSRNWLPGPCYRQQRVSKTSADNNN
SEYSWTGATKYHLNGRDSLVNPGPAMASHKDDEEKFFPQSGVLIFGKQGSEKTNVDIEKVMITDEEEIRTTN
PVATEQYGSVSTNLQRGNIQAATADVNTQGVLPGMVWQDRDVYLQGPIWAKIPHTDGHFHPSPLMGGFGL
KHPPPQILIKNTPVPANPSTTFSAAKFASFITQYSTGQVSVEIEWELQKENSKRWNPEIQYTSNYNKSVNVDF
TVDTNGVYSEPRPIGTRYLTRNL
CMRI 19 (SEQ ID NO: 20):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYFGYSTPWGYH
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTQSRLQFSQAGASDIRDQSRNWLPGPCYRQQRVSKTSADNDN
SEYSWTGATKYHLNGRDSLVNPGPAMASHKDDEEKFFPQSGVLIFGKQGSEKTNVDIEKVMITDEEEIRTTN
PVATEQYGSVSTNLQRGNRQAATADVNTQGVLPGMVWQDRDVYLQGPIWAKIPHTDGHFHPSPLMGGFGL
KHPPPQILIKNTPVPANPSTTFSAAKFASFITQYSTGQVSVEIEWELQKENSKRWNPEIQYTSNYNKSVNVDF
TVDTNGVYSEPRPIGTRYLTRNL
CMRI 20 (SEQ ID NO: 21):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYFGYSTPWGYH
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTQSRLQFSQAGASDIRDQSRNWLPGPCYRQQRVSKTSADNNN
SEYSWTGATKYHLNGRDSLVNPGPAMASHKDDEEQFFPQSGVLIFGKQGSEKTNVDIEKVMITDEEEIRTTN
PVATEQYGSVSTNLQRGNRQAATADVNTQGVLPGMVWQDRDVYLQGPIWAKIPHTDGHFHPSPLMGGFGL
KHPPPQILIKNTPVPANPSTTFSAAKFASFITQYSTGQVSVEIEWELQKENSKRWNPEIQYTSNYNKSVNVDF
TVDTNGVYSEPRPIGTRYLTRNL
CMRI 21 (SEQ ID NO: 22):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTQSRLQFSQAGASDIRDQSRNWLPGPCYRQQRVSKTSADNNN
SEYSWTGATKYHLNGRDSLVNPGPAMASHKDDEEKFFPQSGVLIFGKQGSEKTNVDIEKVMITDEEEIRTTN
PVATEQYGSVSTSLQRGNRQAATADVNTQGVLPGMVWQDRDVYLQGPIWAKIPHTDGHFHPSPLMGGFGL
KHPPPQILIKNTPVPANPSTTFSAAKFASFITQYSTGQVSVEIEWELQKENSKRWNPEIQYTSNYNKSVNVDF
TVDTNGVYSEPRPIGTRYLTRNL
CMRI 22 (SEQ ID NO: 23):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPAEPDSSSGTGKAGNQPARKRLNFGQTGDADSVPDPQPLGQPPAAPSGLGSTTMATGSGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQSNSGTLQQSRLLFSQAGPTSMSLQAKNWLPGPCYRQQRLSKQANDNNN
SNFPWTAATKYHLNGRDSLVNPGPAMASHKDDEEKFFPMHGTLIFGKQGTNANDADLDNVMITDEEEIRTT
NPVATEQYGYVSNNLQNSNTGPTTGTVNHQGALPGMVWQDRDVYLQGPIWAKIPHTDGHFHPSPLMGGFG
LKHPPPQIMIKNTPVPANPPTNFSSAKFASFITQYSTGQVSVEIEWELQKENSKRWNPEIQYTSNYNKSVNVD
FTVDTNGVYSEPRPIGTRYLTRNL
CMRI 23 (SEQ ID NO: 24):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYGYSTPWGYH
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSKTNAPSGTTTMSRLQFSQAGASDIRDQSRNWLPGPCYRQQRVSKTAADNNN
SDYSWTGATKYHLNGRDSLVNPGTAMASHKDDEEKYFPQSGVLIFGKQDSGKTNVDIEKVMITDEEEIRTT
NPVATEQYGSVSTNLQSGNTQAATSDVNTQGVLPGMVWQDRDVYLQGPIWAKIPHTDGHFHPSPLMGGFG
LKHPPPQILIKNTPVPANPSTTFSAAKFASFITQYSTGQVSVEIEWELQKENSKRWNPEIQYTSNYNKSVNVD
FTVDTNGVYSEPRPIGTRYLTRNL
CMRI 24 (SEQ ID NO: 25):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPAEPDSSSGTGKAGQQPAKKRLNFGQTGDSDSVPDPQPLGEPPAAPTSLGSTTMASGGGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQSASGTVQQSRLLFSQAGPTSMSLQAKNWLPGPCYRQQRLSKQANENNN
SNFPWTAATKYHLNGRDSLVNPGPPMASHKDDEEKFFPMHGTLIFGKEGTNANNAELENVMITDEEEIRTTN
PVATEQYGNVSNNLQNSNTAPSTGTVNHQGALPGMVWQDRDVYLQGPIWAKIPHTDGHFHPSPLMGGFGL
KHPPPQIMIKNTPVPANPPTNFSAAKFASFITQYSTGQVSVEIEWELQKENSKRWNPEIQYTSNYNKSVNVDF
TVDTNGVYSEPRPIGTRYLTRNL
CMRI 25 (SEQ ID NO: 26):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPAEPDSSSGTGKAGQQPARKRLNFGQTGDADSVPDPQPLGQPPAAPSGLGSTTMATGSGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQSNSGTLQQSRLLFSQAGPTSMSLQAKNWLPGPCYRQQRLSKQANDNNN
SNFPWTAATKYHLNGRDSLVNPGPAMASHKDDEEKFFPMHGTLIFGKQGTNANDADLDNVMITDEEEIRTT
NPVATEQYGYVSNNLQNSNTGPTTGTVNHQGALPGMVWQDRDVYLQGPIWAKIPHTDGHFHPSPLMGGFG
LKHPPPQIMIKNTPVPANPPTNFSSAKFASFITQYSTGQVSVEIEWELQKENSKRWNPEIQYTSNYNKSVNVD
FTVDTNGVYSEPRPIGTRYLTRNL
CMRI 26 (SEQ ID NO: 27):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQESLKEDTSFGGNLGRAVFQAKKRVLEPLGLVEEPVKTAPGKK
RPVEHSPAEPDSSSGTGKAGQQPAKKRLNFGQTGDSDSVPDPQPLGEPPAAPTSLGSTTMASGGGAPVAD
NNEGADGVGNSSGNWHCDSQWLGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFQFSYTFEDVPFHSSYAH
SQSLDRLMNPLIDQYLYYLNKTQSASGTVQQSRLLFSQAGPTSMSLQAKNWLPGPCYRQQRLSKQANENNN
SNFPWTAATKYHLNGRDSLVNPGPPMASHKDDEEKFFPMHGTLIFGKEGTNANNAELENVMITDEEEIRTTN
PVATEQYGNVSNNLQNSNTAPSTGTVNHQGALPGMVWQDRDVYLQGPIWAKIPHTDGHFHPSPLMGGFGL
KHPPPQIMIKNTPVPANPPTNFSAAKFASFITQYSTGQVSVEIEWELQKENSKRWNPEIQYTSNYNKSVNVDF
TVDTNGVYSEPRPIGTRYLTRNL
CMRI 27 (SEQ ID NO: 62):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYGYSTPWGYH
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTQSRLQFSQAGASDIRDQSRNWLPGPCYRQQRVSKTSADNNN
SEYSWTGATKYHLNGRDSLVNPGPAMASHKDDEEKFFPQSGVLIFGKQGSEKTNVDIEKVMITDEEEIRTTN
PVATEQYGSVSTNLQRGNTQAATADVNTQGVLPGMVWQDRDVYLQGPIWAKIPHTDGHFHPSPLMGGFGL
KHPPPQILIKNTPVPANPSTTFSAAKFASFITQYSTGQVSVEIEWELQKENSKRWNPEIQYTSNYNKSVNVDF
TVDTNGVYSEPRPIGTRYLTRNL
CMRI 28 (SEQ ID NO: 63):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYFGYSTPWGYH
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTQSRLQFSQAGASDIRDQSRNWLPGPCYRQQQVSKTSADNNN
SEYSWTGATKYHLNGRDSLVNPGPAMASHKDDEEKFFPQSGVLIFGKQGSEKTNVDIEKVMITDEEEIRTTN
PVATEQYGSVSTNLQRGNRQAATADVNTQGVLPGMVWQDRDVYLQGPIWAKIPHTDGHFHPSPLMGGFGL
KHPPPQILIKNTPVPANPSTTFSAAKFASFITQYSTGQVSVEIEWELQKENSKRWNPEIQYTSNYNKSVNVDF
TVDTNGVYSEPRPIGTRYLTRNL
CMRI 29 (SEQ ID NO: 64):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYFGYSTPWGYF
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTQSRLQFSQAGASDIRDQSRNWLPGPCYRQQRVSKTSADNNN
SEYSWTGATKYHLNGRDSLVNPGPAMASHKDDEEEFFPQSGVLIFGKQGSEKTNVDIEKVMITDEEEIRTTN
PVATEQYGSVSTNLQRGNRQAATADVNTQGVLPGMVWQDRDVYLQGPIWAKIPHTDGHFHPSPLMGGFGL
KHPPPQILIKNTPVPANPSTTFSAAKFASFITQYSTGQVSVEIEWELQKENSKRWNPEIQYTSNYNKSVNVDF
TVDTNGVYSEPRPIGTRYLTRNL
CMRI 30 (SEQ ID NO: 65):
MAADGYLPDWLEDTLSEGIRQWWKLKPGPPPPKPAERHKDDSRGLVLPGYKYLGPFNGLDKGEPVNEADAA
ALEHDKAYDRQLDSGDNPYLKYNHADAEFQERLKEDTSFGGNLGRAVFQAKKRVLEPLGLVEEPVKTAPGKK
RPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYFGYSTPWGYH
DFNRFHCHFSPRDWQRLINNNWGFRPKRLNFKLFNIQVKEVTQNDGTTTIANNLTSTVQVFTDSEYQLPYVL
GSAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTQSRLQFSQAGASDIRDQSRNWLPGPCYRQQRVSKTSADNNN
SEYSWAGATKYHLNGRDSLVNPGPAMASHKDDEEKFFPQSGVLIFGKQGSEKTNVDIEKVMITDEEEIRTTN
PVATEQYGSVSTNLQRGNRQAATADVDTQGVLPGMVWQDRDVYLQGPIWAKIPHTDGHFHPSPLMGGFGL
KHPPPQILIKNTPVPANPSTTFSAAKFASFITQYSTGQVSVEIEWELQKENSKRWNPEIQYTSNYNKSVNVDF
TVDTNGVYSEPRPIGTRYLTRNL
CMRI 31 (SEQ ID NO: 71):
MAADGYLPDWLEDNLSEGIREWWALQPGAPKPKANQQHQDNARGLVLPGYKYLGPGNGLDKGEPVNAAD
AAALEHDKAYDQQLKAGDNPYLKYNHADAEFQERLKEDTSFGGNLGRAVFQAKKRLLEPLGLVEEAAKTAPG
KKRPVDQSPQEPDSSSGVGKSGKQPARKRLNFGQTGDSESVPDPQPLGEPPAAPTSLGSNTMASGGGAPM
ADNNEGADGVGNSSGNWHCDSQWLGDRVITTSTRTWALPTYNNHLYKQISSQSGASNDNHYFGYSTPWG
YFDFNRFHCHFSPRDWQRLINNNWGFRPKKLSFKLFNIQVKEVTQNDGTTTIANNLTSTVQVFTDSEYQLPY
VLGSAHQGCLPPFPADVFMVPQYGYLTLNNGSQAVGRSSFYCLEYFPSQMLRTGNNFQFSYTFEDVPFHSSY
AHSQSLDRLMNPLIDQYLYYLNRTQGTTSGTTNQSRLLFSQAGPQSMSLQARNWLPGPCYRQQRLSKTAND
NNNSNFPWTAASKYHLNGRDSLVNPGPAMASHKDDEEKFFPMHGNLIFGKEGTTASNAELDNVMITDEEEI
RTTNPVATEQYGTVANSLQSSNTAPTTRTVNDQGALPGMVWQDRDVYLQGPIWAKIPHTDGHFHPSPLMG
GFGLKHPPPQIMIKNTPVPANPPTTFSPAKFASFITQYSTGQVSVEIEWELQKENSKRWNPEIQYTSNYNKSV
NVDFTVDTNGVYSEPRPIGTRYLTRPL
CMRI 32 (SEQ ID NO: 72):
MAADGYLPDWLEDNLSEGIREWWALQPGAPKPKANQQHQDNARGLVLPGYKYLGPGNGLDKGEPVNAAD
AAALEHDKAYDQQLKAGDNPYLKYNHADAEFQERLKEDTSFGGNLGRAVFQAKKRLLEPLGLVEEAAKTAPG
KKRPVDQSPQEPDSSSGVGKSGKQPARKRLNFGQTGDSESVPDPQPLGEPPAAPTSLGSNTMASGGGAPM
ADNNEGADGVGNSSGNWHCDSQWLGDRVITTSTRTWALPTYNNHLYKQISSQSGASNDNHYFGYSTPWG
YFDFNRFHCHFSPRDWQRLINNNWGFRPKKLSFKLFNIQVKEVTQNDGTTTIANNLTSTVQVFTDSEYQLPY
VLGSAHQGCLPPFPADVFMVPQYGYLTLNNGSQAVGRSSFYCLEYFPSQMLRTGNNFQFSYTFEDVPFHSSY
AHSQSLDRLMNPLIDQYLYYLNRTQGTTSGTTNQSRLLFSQAGPQSMSLQARNWLPGPCYRQQQLSKTAND
NNNSNFPWTAASKYHLNGRDSLVNPGPAMASHKDDEEKFFPMHGNLIFGKEGTTASNAELDNVMITDEEEI
RTTNPVATEQYGTVANNLQSSNTAPTTRTVNDQGALPGMVWQDRDVYLQGPIWAKIPHTDGHFHPSPLMG
GFGLKHPPPQIMIKNTPVPANPPTTFSPAKFASFITQYSTGQVSVEIEWELQKENSKRWNPEIQYTSNYNKSV
NVDFTVDTNGVYSEPRPIGTRYLTRPL
CMRI 33 (SEQ ID NO: 73):
MAADGYLPDWLEDNLSEGIREWWALQPGAPKPKANQQHQDNARGLVLPGYKYLGPGNGLDKGEPVNAAD
AAALEHDKAYDQQLKAGDNPYLKYNHADAEFQERLKEDTSFGGNLGRAVFQAKKRLLEPLGLVEEAAKTAPG
KKRPVDQSPQEPDSSSGVGKSGKQPARKRLNFGQTGDSESVPDPQPLGEPPAAPTSLGSNTMASGGGAPM
ADNNEGADGVGNSSGNWHCDSQWLGDRVITTSTRTWALPTYNNHLYKQISSQSGASNDNHYFGYSTPWG
YFDFNRFHCHFSPRDWQRLINNNWGFRPKKLSFKLFNIQVKEVTQNDGTTTIANNLTSTVQVFTDSEYQLPY
VLGSAHQGCLPPFPADVFMVPQYGYLTLNNGSQAVGRSSFYCLEYFPSQMLRTGNNFQFSYTFEDVPFHSSY
AHSQSLDRLMNPLIDQYLYYLNRTQGTTSGTTNQSRLLFSQAGPQSMSLQARNWLPGPCYRQQRLSKTAND
NNNSNFPWTAASKYHLNGRDSLVNPGPAMASHKDDEEKFFPMHGNLIFGKEGTTASNAELDNVMITDEEEI
RTTNPVATEQYGTVANNLQSSNTAPTTGTVNDQGALPGMVWQDRDVYLQGPIWAKIPHTDGHFHPSPLMG
GFGLKHPPPQIMIKNTPVPANPPTTFSPAKFASFITQYSTGQVSVEIEWELQKENSKRWNPEIQYTSNYNKSV
NVDFTVDTNGVYSEPRPIGTRYLTRPL
CMRI 34 (SEQ ID NO: 74):
MAADGYLPDWLEDNLSEGIREWWALQPGAPKPKANQQHQDNARGLVLPGYKYLGPGNGLDKGEPVNAAD
AAALEHDKAYDQQLKAGDNPYLKYNHADAEFQERLKEDTSFGGNLGRAVFQAKKRLLEPLGLVEEAAKTAPG
KKRPVDQSPQEPDSSSGVGKSGKQPARKRLNFGQTGDSESVPDPQPLGEPPAAPTSLGSNTMASGGGAPM
ADNNEGADGVGNSSGNWHCDSQWLGDRVITTSTRTWALPTYNNHLYKQISSQSGASNDNHYFGYSTPWG
YFDFNRFHCHFSPRDWQRLINNNWGFRPKKLSFKLFNIQVKEVTQNDGTTTIANNLTSTVQVFTDSEYQLPY
VLGSAHQGCLPPFPADVFMVPQYGYLTLNNGSQAVGRSSFYCLEYFPSQMLRTGNNFQFSYTFEDVPFHSSY
AHSQSLDRLMNPLIDQYLYYLNRTQGTTSGTTNQSRLLFSQAGPQSMSLQARNWLPGPCYRQQRLSKTAND
NNNSNFPWTAASKYHLNGRDSLVNPGPAMASHKDDEEKFFPMHGNLIFGKEGTTASNAELDNVMITDEEEI
RTTNPVATEQYGTVANNLQSSNTAPTTETVNHQGALPGMVWQDRDVYLQGPIWAKIPHTDGHFHPSPLMG
GFGLKHPPPQIMIKNTPVPANPPTTFSPAKFASFITQYSTGQVSVEIEWELQKENSKRWNPEIQYTSNYNKSV
NVDFTVDTNGVYSEPRPIGTRYLTRPL

1. A method for producing a modified AAV vector for use in transducing human hepatocytes in vivo, or for enhancing the in vivo human hepatocyte transduction efficiency of an AAV vector, comprising:
a) identifying a reference capsid polypeptide for transducing human hepatocytes in vivo;
b) modifying the sequence of the reference capsid polypeptide to introduce one or more amino acid substitutions that alter the affinity of the capsid polypeptide for heparin, to thereby produce a modified capsid polypeptide; and
c) vectorising the modified capsid polypeptide to thereby produce a modified AAV vector;
wherein at least one of the following applies:
i) the modified AAV vector is characterized in that it elutes from a heparin affinity chromatography medium with a NaCl concentration of no more than 450 mM or with a conductivity of no more than 41 ms/cm; and has an in vivo transduction efficiency of human hepatocytes that is enhanced compared to a reference AAV vector having a capsid comprising the reference capsid polypeptide,
ii) the modified AAV vector is characterized in that it elutes from a heparin affinity chromatography medium with a NaCl concentration of no more than 275 mM or with a conductivity of no more than 25 ms/cm; and has an in vivo transduction efficiency of human hepatocytes that is enhanced compared to a reference AAV vector having a capsid comprising the reference capsid polypeptide, and
iii) the modified AAV vector is characterized in that it elutes from a heparin affinity chromatography medium with a NaCl concentration of no more than 330 mM or with a conductivity of no more than 30 ms/cm; and has an in vivo transduction efficiency of hepatocytes that is enhanced compared to a reference AAV vector having a capsid comprising the reference capsid polypeptide.

2-76. (canceled)

77. The method of claim 1 when (i) applies, wherein step b) comprises modifying the sequence of the reference capsid polypeptide to introduce one or more amino acid substitutions at an amino acid position corresponding to a position selected from among position 482, 484, 487, 496, 503, 532, 582, 585, 588, 589 or 596 of SEQ ID NO:1.

78. The method of claim 1 when (i) applies, wherein step b) comprises modifying the sequence of the reference capsid polypeptide to introduce two or more amino acid substitutions at amino acid positions selected from among those corresponding positions 482, 496, 503, 532, 585, 588 or 596 of SEQ ID NO:1, optionally wherein the two or more amino acid substitutions are at the amino acid positions corresponding to positions 482 and 496; 482 and 532; 482, 496 and 532; 482 and 585; 482, 503 and 596; 496, 588 and 596; 496, 532, 585, 588 and 596; or 503, 585, 588 and 596 of SEQ ID NO:1.

79. The method of claim 1, when ii) applies, wherein step b) comprises modifying the sequence of the reference capsid polypeptide to introduce one or more amino acid substitutions at an amino acid position corresponding to a position selected from among position 476, 485, 488, 533, 594 or 598 of SEQ ID NO:56.

80. The method of claim 1, when ii) applies, wherein step b) comprises modifying the sequence of the reference capsid polypeptide to introduce at least two amino acid substitutions at amino acid positions corresponding to positions 594 and 598 of SEQ ID NO:56.

81. The method of claim 1 when iii) applies, wherein step b) comprises modifying the sequence of the reference capsid polypeptide to introduce one or more amino acid substitutions at an amino acid position corresponding to a position selected from among 482, 485, 525, 528 or 530 of SEQ ID NO:55.

82. The method of claim 1 when iii) applies, wherein the one or more amino acid substitutions is selected from among:
substitution of the amino acid residue at the position corresponding to position 482 of SEQ ID NO:55 with a glutamine (Q) or asparagine (N);
substitution of the amino acid residue at the position corresponding to position 485 of SEQ ID NO:55 with a glutamine (Q) or asparagine (N);
substitution of the amino acid residue at the position corresponding to position 525 of SEQ ID NO:55 with a glutamic acid (E) or glutamine (Q);
substitution of the amino acid residue at the position corresponding to position 528 of SEQ ID NO:55 with a glutamic acid (E) or glutamine (Q); or
substitution of the amino acid residue at the position corresponding to position 530 of SEQ ID NO:55 with a glutamic acid (E) or glutamine (Q).

83. A method for identifying a candidate AAV vector that is suitable for in vivo transduction of human hepatocytes, comprising assessing the heparin binding capacity of a test AAV vector and comparing the heparin binding capacity of the test AAV vector to the heparin binding capacity of a reference AAV vector, wherein at least one of a) to c) applies:
a) the test AAV vector is an AAV2 or AAV2-like vector comprising a capsid polypeptide comprising the sequence set forth in SEQ ID NO:1 or a sequence having at least or about 90% sequence identity thereto; and
the reference AAV vector elutes from a heparin affinity chromatography medium with a NaCl concentration at least 450 mM or with a conductivity of at least 41 ms/cm;
b) the test AAV vector is an AAV3B or AAV3B-like vector comprising a capsid polypeptide comprising the sequence set forth in SEQ ID NO:56 or a sequence having at least or about 90% sequence identity thereto; and
the reference AAV vector elutes from a heparin affinity chromatography medium with a NaCl concentration at least 275 mM or with a conductivity of at least 25 ms/cm; or
c) the test AAV vector is an AAV13 or AAV13-like vector comprising a capsid polypeptide comprising the sequence set forth in SEQ ID NO:55 or a sequence having at least or about 90% sequence identity thereto; and
the reference AAV vector elutes from a heparin affinity chromatography medium with a NaCl concentration at least 330 mM or with a conductivity of at least 30 ms/cm;
wherein when the test AAV vector has a heparin binding capacity below the reference AAV vector, the test AAV vector is identified as a candidate AAV vector that is suitable for in vivo transduction of human hepatocytes.

84. An AAV vector that is produced by the method of claim 1.

85. A method for delivering a heterologous coding sequence to a hepatocyte in a human subject, comprising administering to human subject an AAV vector comprising a heterologous coding sequence, wherein the AAV vector comprises a capsid comprising a wild-type AAV2 capsid polypeptide comprising an amino acid sequence having at least 92% sequence identity to the polypeptide set forth in SEQ ID NO:5.

86. A method for treating or inhibiting a liver-associated disease in a human subject by administering to the human subject an AAV vector comprising:
a) a heterologous coding sequence;
b) a capsid comprising a wild-type AAV2 capsid polypeptide comprising an amino acid sequence having at least 92% sequence identity to the polypeptide set forth in SEQ ID NO:5;
to deliver the heterologous coding sequence to a hepatocyte in the human subject and effect in vivo expression of the heterologous coding sequence in the hepatocyte, thereby treating or inhibiting the liver-associated disease.

87. An isolated capsid polypeptide, comprising at least one of:
a) the sequence of amino acids set forth in any one of SEQ ID Nos:2-27, 62-65 and 71-74 or a sequence having at least or about 90% sequence identity thereto;
b) the sequence of amino acids at positions 138-735 of any one of SEQ ID NOs:2, 3, 5-11, 19-27 and 62-65, positions 138-734 of SEQ ID NO:4 or 24, positions 138-736 of SEQ ID NO:16 or 71-74, or positions 138-737 of any one of SEQ ID NOs:12-15, 17 and 18; or a sequence having at least or about 90% sequence identity thereto;
c) the sequence of amino acids at positions 203-735 of any one of SEQ ID Nos:2, 3, 5-11, 19-27 and 62-65, positions 203-734 of SEQ ID NO:4, positions 204-736 of SEQ ID NO:16 or 71-74, or positions 204-737 of any one of SEQ ID NOs:12-15, 17 and 18; or a sequence having at least or about 90% sequence identity thereto;
d) a sequence of amino acids comprising one or more amino acid substitutions relative to the AAV2 capsid polypeptide set forth in SEQ ID NO:1 or a polypeptide having at least or about 90% sequence identity to the polypeptide set forth in SEQ ID NO:1, wherein the one or more amino acid substitutions are at an amino acid position selected from among those corresponding to positions 482, 484, 487, 496, 503, 532, 582, 585, 588, 589 and 596 of the AAV2 capsid polypeptide set forth in SEQ ID NO:1;
e) a sequence of amino acids comprising one or more amino acid substitutions relative to the AAV3B capsid polypeptide set forth in SEQ ID NO:56 or a polypeptide having at least or about 90% sequence identity to the polypeptide set forth in SEQ ID NO:56, wherein the one or more amino acid substitutions are at an amino acid position selected from among those corresponding to positions 476, 485, 488, 533, 594 and 598 of the AAV3B capsid polypeptide set forth in SEQ ID NO:56; or
f) a sequence of amino acids comprising one or more amino acid substitutions relative to the AAV13 capsid polypeptide set forth in SEQ ID NO:55 or a polypeptide having at least or about 90% sequence identity to the polypeptide set forth in SEQ ID NO:55, wherein the one or more amino acid substitutions are at an amino acid position selected from among those corresponding to positions 482, 485, 525, 528 and 530 of the AAV13 capsid polypeptide set forth in SEQ ID NO:55.

88. An AAV vector, comprising the capsid polypeptide of claim 49.

89. An isolated nucleic acid molecule encoding the capsid polypeptide of claim 49.

90. A vector comprising the nucleic acid molecule of claim 69.

91. A host cell, comprising the AAV vector of claim 66, the nucleic acid molecule of claim 69.

92. A method for introducing a heterologous coding sequence into a host cell, comprising contacting a host cell with the AAV vector of claim 68.

93. A method for producing an AAV vector, comprising culturing a host cell comprising a nucleic acid molecule encoding the capsid polypeptide of claim 45, an AAV rep gene, a heterologous coding sequence flanked by AAV inverted terminal repeats, and helper functions for generating a productive AAV infection, under conditions suitable to facilitate assembly of an AAV vector comprising a capsid comprising the capsid polypeptide of claim 45, wherein the capsid encapsidates the heterologous coding sequence.

94. The method of claim 75, wherein the host cell is a human hepatocyte.

95. A host cell, comprising at least one of:
a) the isolated nucleic acid molecule of claim 69, and
b) a vector comprising the nucleic acid molecule.
